SEQUENCE LISTING (347 sequences)

SEQ ID NO: 1 - DNA, 20 bp, Artificial Sequence
  Primer GlnPr269
caggggggcggagcctatgg

SEQ ID NO: 2 - DNA, 43 bp, Artificial Sequence
  Primer GlnPr497
gcccccctcccgcgaggagatgaccaagaaccaggtgtccctg

SEQ ID NO: 3 - DNA, 34 bp, Artificial Sequence
  Primer GlnPr498
tttctagattcatcacttgccgggggacaggctc

SEQ ID NO: 4 - DNA, 31 bp, Artificial Sequence
  Primer GlnPr501
tataaagcttccaccatggagagccctgccc

SEQ ID NO: 5 - DNA, 33 bp, Artificial Sequence
  Primer GlnPr502
tatatctagattcatcagcactcgccccggttg

SEQ ID NO: 6 - DNA, 35 bp, Artificial Sequence
  Primer GlnPr1437
ccaaatcgatttatcagcactcgccccggttgaag

SEQ ID NO: 7 - DNA, 35 bp, Artificial Sequence
  Primer GlnPr1500
ccgggatcgatttatcagtgatggtgatggtgatg

SEQ ID NO: 8 - DNA, 32 bp, Artificial Sequence
  Primer GlnPr1502
ggccgatcgatttatcacttgccaggagacag

SEQ ID NO: 9 - DNA, 28 bp, Artificial Sequence
  Primer GlnPr1516
gatgaaagcttggtaggtgatcctcctg

SEQ ID NO: 10 - DNA, 52 bp, Artificial Sequence
  Primer GlnPr1517
atgtattcgaatatctccaccggttgcagctctgagccagggaggagggaag

SEQ ID NO: 11 - DNA, 28 bp, Artificial Sequence
  Primer GlnPr1518
taagctagcccaccatggagagccctgc

SEQ ID NO: 12 - DNA, 49 bp, Artificial Sequence
  Primer GlnPr1519
gaggctcttctgcgtgtagtggttgtgcaaggcctcgtgcatcacgctg

SEQ ID NO: 13 - DNA, 51 bp, Artificial Sequence
  Primer GlnPr1520
ctaccaagctttcatcatttacccggagacagggagaggctcttctgcgtg

SEQ ID NO: 14 - DNA, 92 bp, Artificial Sequence
  Primer GlnPr1521
gggagctggacgggctgtggacgaccatcaccatcttcatcacactcttcctgttaagcgtgtgctacagtgccaccgtcaccttcttcaag

SEQ ID NO: 15 - DNA, 92 bp, Artificial Sequence
  Primer GlnPr1522
gtagtcggggatgatggtctgcttcaggtccaccaccgaggagaagatccacttcaccttgaagaaggtgacggtggcactgtagcacacgc

SEQ ID NO: 16 - DNA, 55 bp, Artificial Sequence
  Primer GlnPr1523
atataccggtggagagctgtgcggaggcgcaggacggggagctggacgggctgtg

SEQ ID NO: 17 - DNA, 59 bp, Artificial Sequence
  Primer GlnPr1524
atatttcgaatcactaggccccctgtccgatcatgttcctgtagtcggggatgatggtc

SEQ ID NO: 18 - DNA, 41 bp, Artificial Sequence
  Primer GlnPr1649
tctccaccggttgcagctctgttccctcctccctcggttag

SEQ ID NO: 19 - DNA, 33 bp, Artificial Sequence
  Primer GlnPr1650
gcgccatcgatacagacatgataagatacattg

SEQ ID NO: 20 - DNA, 36 bp, Artificial Sequence
  Primer GlnPr1651
gcgcgatcgatacttgtttattgcagcttataatgg

SEQ ID NO: 21 - DNA, 54 bp, Artificial Sequence
  Primer GlnPr1689
atatgctagcccaccatgcggagccctgcccagctgctgttcctgctgctgctg

SEQ ID NO: 22 - DNA, 61 bp, Artificial Sequence
  Primer GlnPr1690
tgctgttcctgctgctgctgtggatccctggcaccaacgccgaggtgcagctggtcgagtc

SEQ ID NO: 23 - DNA, 54 bp, Artificial Sequence
  Primer GlnPr1725
atatactagtccaccatgcggagccctgcccagctgctgttcctgctgctgctg

SEQ ID NO: 24 - DNA, 59 bp, Artificial Sequence
  Primer GlnPr1726
ctgttcctgctgctgctgtggatccctggcaccaacgcccaggtgcagctgcagcagtc

SEQ ID NO: 25 - DNA, 58 bp, Artificial Sequence
  Primer GlnPr1727
ctgttcctgctgctgctgtggatccctggcaccaacgccatggagacagacacactcc

SEQ ID NO: 26 - DNA, 70 bp, Artificial Sequence
  Primer GlnPr1735
gacttcgaatcatcaatgatggtggtggtgatgggcgctggcgaggagcaggtcgaacaggagcagcttg

SEQ ID NO: 27 - DNA, 37 bp, Artificial Sequence
  Primer GlnPr1760
ccgcatcgatttatcatttacccgggctcaggctcag

SEQ ID NO: 28 - DNA, 71 bp, Artificial Sequence
  Primer GlnPr1799
atataccggtggaggccctgtggctgggcgtgctgagactgctgctgttcaagctgctcctgttcgacctg

SEQ ID NO: 29 - DNA, 60 bp, Artificial Sequence
  Primer GlnPr1800
gtaagctttcatcatttacccggagacagggagaggctcttctgcgtgaatcggttgtgc

SEQ ID NO: 30 - DNA, 60 bp, Artificial Sequence
  Primer GlnPr1801
gcgcgaagctttcatcatttacccggagacagggagaggctcttctgcgtataatgattg

SEQ ID NO: 31 - DNA, 35 bp, Artificial Sequence
  Primer GlnPr1840
atataccggtggaggccctgtggctgggcgtgctg

SEQ ID NO: 32 - DNA, 46 bp, Artificial Sequence
  Primer GlnPr2099
tcctcggggggagatccaccaccacctgagccagggaggagggaag

SEQ ID NO: 33 - DNA, 31 bp, Artificial Sequence
  Primer GlnPr2100
gcgcttcgaatcatcacagaaacacggtctg

SEQ ID NO: 34 - DNA, 40 bp, Artificial Sequence
  Primer GlnPr2101
tggtggtggatctccccccgaggacccccctgactccaag

SEQ ID NO: 35 - DNA, 48 bp, Artificial Sequence
  Primer GlnPr2102
tcctcggggggagatccaccaccacctgttccctcctccctcggttag

SEQ ID NO: 36 - DNA, 1082 bp, Artificial Sequence
  Construct GSC3899
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcagagctgcaaccggtggagagctgtgcggaggcgcaggacggggagctggacgggctgtggacgaccatcaccatcttcatcacactcttcctgttaagcgtgtgctacagtgccaccgtcaccttcttcaaggtgaagtggatcttctcctcggtggtggacctgaagcagaccatcatccccgactacaggaacatgatcggacagggggcctagtgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 37 - DNA, 1064 bp, Artificial Sequence
  Construct GSC4398
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagggaggagggaacagagctgcaaccggtggagagctgtgcggaggcgcaggacggggagctggacgggctgtggacgaccatcaccatcttcatcacactcttcctgttaagcgtgtgctacagtgccaccgtcaccttcttcaaggtgaagtggatcttctcctcggtggtggacctgaagcagaccatcatccccgactacaggaacatgatcggacagggggcctagtgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 38 - DNA, 1219 bp, Artificial Sequence
  Construct GSC4401
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatacttgtttattgcagcttataatggttacaaataaagcaatagcatcacaaatttcacaaataaagcatttttttcactgcattctagttgtggtttgtccaaactcatcaatgtatcttatcatgtctgtatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcagagctgcaaccggtggagagctgtgcggaggcgcaggacggggagctggacgggctgtggacgaccatcaccatcttcatcacactcttcctgttaagcgtgtgctacagtgccaccgtcaccttcttcaaggtgaagtggatcttctcctcggtggtggacctgaagcagaccatcatccccgactacaggaacatgatcggacagggggcctagtgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 39 - DNA, 1263 bp, Artificial Sequence
  Construct GSC4402
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcagagctgcaaccggtggagagctgtgcggaggcgcaggacggggagctggacgggctgtggacgaccatcaccatcttcatcacactcttcctgttaagcgtgtgctacagtgccaccgtcaccttcttcaaggtgaagtggatcttctcctcggtggtggacctgaagcagaccatcatccccgactacaggaacatgatcggacagggggcctagtgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggggcaggataatatatggtagggttcatagccagagtaacctttttttttaatttttattttattttatttttgagatcag

SEQ ID NO: 40 - DNA, 977 bp, Artificial Sequence
  Construct GSC5854
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcagagctgcaaccggtggaggccctgtggctgggcgtgctgagactgctgctgttcaagctgctcctgttcgacctgctcctcgccagcgcccatcaccaccaccatcattgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 41 - DNA, 959 bp, Artificial Sequence
  Construct GSC5855
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagggaggagggaacagagctgcaaccggtggaggccctgtggctgggcgtgctgagactgctgctgttcaagctgctcctgttcgacctgctcctcgccagcgcccatcaccaccaccatcattgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 42 - DNA, 1114 bp, Artificial Sequence
  Construct GSC5857
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatacttgtttattgcagcttataatggttacaaataaagcaatagcatcacaaatttcacaaataaagcatttttttcactgcattctagttgtggtttgtccaaactcatcaatgtatcttatcatgtctgtatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcagagctgcaaccggtggaggccctgtggctgggcgtgctgagactgctgctgttcaagctgctcctgttcgacctgctcctcgccagcgcccatcaccaccaccatcattgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 43 - DNA, 1158 bp, Artificial Sequence
  Construct GSC5856
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcagagctgcaaccggtggaggccctgtggctgggcgtgctgagactgctgctgttcaagctgctcctgttcgacctgctcctcgccagcgcccatcaccaccaccatcattgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggggcaggataatatatggtagggttcatagccagagtaacctttttttttaatttttattttattttatttttgagatcag

SEQ ID NO: 44 - DNA, 1076 bp, Artificial Sequence
  Construct GSC6030
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcagagctgcaaccggtggagagctgtgcggaggcgcaggacggggagctggacgccctgtggctgggcgtgctgagactgctgctgttcaagctgctcctgttcgacctgctcctcaccttcttcaaggtgaagtggatcttctcctcggtggtggacctgaagcagaccatcatccccgactacaggaacatgatcggacagggggcctagtgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 45 - DNA, 1058 bp, Artificial Sequence
  Construct GSC6048
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagggaggagggaacagagctgcaaccggtggagagctgtgcggaggcgcaggacggggagctggacgccctgtggctgggcgtgctgagactgctgctgttcaagctgctcctgttcgacctgctcctcaccttcttcaaggtgaagtggatcttctcctcggtggtggacctgaagcagaccatcatccccgactacaggaacatgatcggacagggggcctagtgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 46 - DNA, 1213 bp, Artificial Sequence
  Construct GSC6047
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatacttgtttattgcagcttataatggttacaaataaagcaatagcatcacaaatttcacaaataaagcatttttttcactgcattctagttgtggtttgtccaaactcatcaatgtatcttatcatgtctgtatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcagagctgcaaccggtggagagctgtgcggaggcgcaggacggggagctggacgccctgtggctgggcgtgctgagactgctgctgttcaagctgctcctgttcgacctgctcctcaccttcttcaaggtgaagtggatcttctcctcggtggtggacctgaagcagaccatcatccccgactacaggaacatgatcggacagggggcctagtgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 47 - DNA, 1257 bp, Artificial Sequence
  Construct GSC6046
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcagagctgcaaccggtggagagctgtgcggaggcgcaggacggggagctggacgccctgtggctgggcgtgctgagactgctgctgttcaagctgctcctgttcgacctgctcctcaccttcttcaaggtgaagtggatcttctcctcggtggtggacctgaagcagaccatcatccccgactacaggaacatgatcggacagggggcctagtgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggggcaggataatatatggtagggttcatagccagagtaacctttttttttaatttttattttattttatttttgagatcag

SEQ ID NO: 48 - DNA, 1738 bp, Artificial Sequence
  Construct GSC314
atggagagccctgcccagctgctgttcctgctgctgctgtggctgcctgacacccaggccgaggtgcagctggtcgagtctggcggcggactggtgcagcctggcggctccctgcggctgtcctgcgccgcctccggcttcaacatcaaggacacctacatccactgggtgcggcaggcccctggcaagggcctggagtgggtggcccggatctaccctaccaacggctacaccagatacgccgactccgtgaagggccggttcaccatctccgccgacacctccaagaacaccgcctacctgcagatgaactccctgcgggccgaggacaccgccgtgtactactgctccagatggggaggagatggcttctacgccatggactactggggccagggcaccctggtgaccgtgtcctccgcctccaccaagggccccagcgtgttccccctggcccccagcagcaagtctaccagcggcggcacagcagccctgggatgcctggtgaaggactacttccccgagcccgtgaccgtgagctggaacagcggagccctgacctccggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccctccctgtcctgctcctgagctgctcggcggaccctccgtgttcctgttcccccccaagcccaaggacaccctgatgatcagcaggacccccgaggtgacctgcgtggtggtggacgtgagccacgaggacccagaggtgaagttcaactggtacgtggacggcgtggaggtgcacaacgccaagaccaagcccagagaggaacagtacaacagcacctacagggtggtgtccgtgctgaccgtgctgcaccaggactggctgaacggcaaagagtacaagtgcaaggtctccaacaaggccctgccagcccccatcgagaaaaccatcagcaaggccaagggccagccacgggagccccaggtgtacaccctgcccccctcccgcgaggagatgaccaagaaccaggtgtccctgacatgtctggtgaaaggcttctaccccagcgacatcgccgtggagtgggagagcaacggccagcccgagaacaactacaagaccacccccccagtgctggacagcgacggcagcttcttcctgtacagcaagctgaccgtggacaagagcaggtggcagcagggcaacgtgttcagctgcagcgtgatgcacgaggccctgcacaaccactacacccagaagagcctgagcctgtcccccggcaagtgatgaatctagaggtacccccggggggcccctcgagttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 49 - DNA, 1410 bp, Artificial Sequence
  Construct GSC5644
atgcggagccctgcccagctgctgttcctgctgctgctgtggatccctggcaccaacgcccaggtgcagctgcagcagtctggcgccgaactggccagaccaggcgccagcgtgaagatgagctgcaaggccagcggctacaccttcacccggtacaccatgcactgggtgaaacagcggcctggacagggcctggaatggatcggctacatcaaccccagccggggctacaccaactacaaccagaagttcaaggacaaggccaccctgaccaccgacaagagcagcagcaccgcctacatgcagctgagcagcctgaccagcgaggacagcgccgtgtactactgcgcccggtactacgacgaccactactgcctggactactggggccagggcaccaccctgaccgtgagcagcgcgtcgaccaagggccccagcgtgttcccgctagcccccagcagcaagagcaccagcggcggcacagccgccctgggctgcctggtgaaggactacttccccgagcccgttaccgtgtcctggaactctggagccctgacctccggcgtgcacaccttccccgccgtgctccagagcagcggcctgtacagcctgagcagcgtggtgacagtgcccagcagcagcctgggaacccagacctacatctgcaacgtgaaccacaagcccagcaacaccaaggtggacaagaaggtggagcccaagagctgcgacaaaactcacacctgcccaccctgccctgcccctgagctgctgggcggaccctccgtgttcctgttcccccccaagcccaaggacaccctgatgatcagccggacccccgaggtgacctgcgtggtggtggacgtgagccacgaggaccctgaggtgaagttcaattggtacgtggacggcgtggaggtgcacaacgccaagaccaagccccgggaggaacagtacaacagcacctaccgggtggtgtccgtgctgaccgtgctgcaccaggactggctgaacggcaaggaatacaagtgcaaggtctccaacaaggccctgcctgcccccatcgaaaagaccatcagcaaggccaagggccagccgcgcgaaccgcaggtgtacaccctgccccccagccgggaagagatgaccaagaaccaggtgaaactggtgtgcctggtgacaggcttctaccccagcgatatcgccgtggaatgggagagcagcggccagcctgagaacaactactacaccaccccccccatgctggacagcgacggcagcttcagcctggtgtcctggctgaacgtggacaagagccggtggcagcagggcaacatcttcagctgcagcgtgatgcacgaggccctgcacaaccggttcacccagaagtccctgagcctgagcccgggtaaatga

SEQ ID NO: 50 - DNA, 1074 bp, Artificial Sequence
  Construct GSC5537
gggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcagagctgcaaccggtggagagctgtgcggaggcgcaggacggggagctggacgggctgtggacgaccatcaccatcttcatcacactcttcctgttaagcgtgtgctacagtgccaccgtcaccttcttcaaggtgaagtggatcttctcctcggtggtggacctgaagcagaccatcatccccgactacaggaacatgatcggacagggggcctagtgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 51 - DNA, 1488 bp, Artificial Sequence
  Construct GSC5608
atgcggagccctgcccagctgctgttcctgctgctgctgtggatccctggcaccaacgccgaggtgcagctggtcgagtctggcggcggactggtgcagcctggcggctccctgcggctgtcctgcgccgcctccggcttcaacatcaaggacacctacatccactgggtgcggcaggcccctggcaagggcctggagtgggtggcccggatctaccctaccaacggctacaccagatacgccgactccgtgaagggccggttcaccatctccgccgacacctccaagaacaccgcctacctgcagatgaactccctgcgggccgaggacaccgccgtgtactactgctccagatggggaggagatggcttctacgccatggactactggggccagggcaccctggtgaccgtgtcctccggtggcggtggcagcggcggtggtggttccggaggcggcggttctgacatccagatgacccagtccccctccagcctgtctgcctccgtgggcgaccgggtgaccatcacctgccgggcctcccaggacgtgaacaccgccgtggcctggtatcagcagaagcctggcaaggcccctaagctgctgatctactccgcctccttcctgtactccggcgtgccttcccggttctccggctcccggtccggcaccgacttcaccctgaccatctcctccctgcagcctgaggacttcgccacctactactgccagcagcactacaccacccctcctaccttcggccagggcaccaaggtggagatcaagaggggaggaggaggtaccgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaaggacagcccagggaacctgaggtggccacattcccacctagccgggacgagctgacaaaaaatcaggtcaccctcgtctgtctcgtgaccggcttttacccttccgacattgccgtggaatgggaatccaatgggcagcccgagaacaattacaagacagacccccccctgctggaatccgatggcagcttcgccctgagcagccggctgcgggtggacaagtccagatggcagcaggggaatgtcttttcctgctccgtcatgcatgaagccctccacaatcattatacacagaaaagcctgagcctgtctcctggcaagtga

SEQ ID NO: 52 - DNA, 1074 bp, Artificial Sequence; identical to SEQ ID NO: 50
  Construct GSC5538

SEQ ID NO: 53 - DNA, 1076 bp, Artificial Sequence
  Construct GSC7057
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagggaggagggaacaggtggtggtggatctccccccgaggacccccctgactccaagaataccctggtgctgttcggcgctggcttcggcgccgtgattaccgtggtcgtgatcgtcgtgattatcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 54 - DNA, 1231 bp, Artificial Sequence
  Construct GSC7058
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatacttgtttattgcagcttataatggttacaaataaagcaatagcatcacaaatttcacaaataaagcatttttttcactgcattctagttgtggtttgtccaaactcatcaatgtatcttatcatgtctgtatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccgtgattaccgtggtcgtgatcgtcgtgattatcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 55 - DNA, 1094 bp, Artificial Sequence
  Construct GSC7056
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccgtgattaccgtggtcgtgatcgtcgtgattatcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 56 - DNA, 1275 bp, Artificial Sequence
  Construct GSC7059
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccgtgattaccgtggtcgtgatcgtcgtgattatcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggggcaggataatatatggtagggttcatagccagagtaacctttttttttaatttttattttattttatttttgagatcag

SEQ ID NO: 57 - DNA, 1094 bp, Artificial Sequence
  GSC9765
ctgtctccgggcaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccgtgattaccgtggtcgtgatcgtcgtgattatcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 58 - DNA, 1094 bp, Artificial Sequence
  GSC9764
ctgtctcccggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccgtgattaccgtggtcgtgatcgtcgtgattatcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 59 - DNA, 1076 bp, Artificial Sequence
  GSC9949
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgtctccttctgggacaggtggtggtggatctccccccgaggacccccctgactccaagaataccctggtgctgttcggcgctggcttcggcgccgtgattaccgtggtcgtgatcgtcgtgattatcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 60 - DNA, 1076 bp, Artificial Sequence
  GSC9768
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccctgggaggattgcacaggtggtggtggatctccccccgaggacccccctgactccaagaataccctggtgctgttcggcgctggcttcggcgccgtgattaccgtggtcgtgatcgtcgtgattatcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 61 - DNA, 1076 bp, Artificial Sequence
  GSC9763
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccctgtccttattgcacaggtggtggtggatctccccccgaggacccccctgactccaagaataccctggtgctgttcggcgctggcttcggcgccgtgattaccgtggtcgtgatcgtcgtgattatcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 62 - DNA, 1076 bp, Artificial Sequence
  GSC9770
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagacctgaggggacaggtggtggtggatctccccccgaggacccccctgactccaagaataccctggtgctgttcggcgctggcttcggcgccgtgattaccgtggtcgtgatcgtcgtgattatcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 63 - DNA, 1094 bp, Artificial Sequence
  GSC9950
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctgctacctggctcaggtggtggtggatctccccccgaggacccccctgactccaagaataccctggtgctgttcggcgctggcttcggcgccgtgattaccgtggtcgtgatcgtcgtgattatcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 64 - DNA, 211 bp, Artificial Sequence
  GeneArt_Seq32
accggtggagagctgtgcggaggcgcaggacggggagctggacgccctgtggctgggcgtgctgagactgctgctgttcaagctgctcctgttcgacctgctcctcaccttcttcaaggtgaagtggatcttctcctcggtggtggacctgaagcagaccatcatccccgactacaggaacatgatcggacagggggcctagtgattcgaa

SEQ ID NO: 65 - DNA, 2009 bp, Homo sapiens
agctttctggggcaggccaggcctgaccttggctttggggcagggagggggctaaggtgaggcaggtggcgccagcaggtgcacacccaatgcccatgagcccagacactggacgctgaacctcgcggacagttaagaacccaggggcctctgcgcctgggcccagctctgtcccacaccgcggtcacatggcaccacctctcttgcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagcaggctcagcgctcctgcctggacgcatcccggctatgcagccccagtccagggcagcaaggcaggccccgtctgcctcttcacccggagcctctgcccgccccactcatgctcagggagagggtcttctggctttttcccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactcccaatcttctctctgcagagcccaaatcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtcctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgagtgcgacggccggcaagccccgctccccgggctctcgcggtcgcacgaggatgcttggcacgtaccccctgtacatacttcccgggcgcccagcatggaaataaagcacccagcgctgccctgggcccctgcgagactgtgatggttctttccacgggtcaggccgagtctgaggcctgagtggcatgagggaggcagagcgggtc

SEQ ID NO: 66 - DNA, 216 bp, Artificial Sequence
  GeneArt_Seq43
ccccccgaggacccccctgactccaagaataccctggtgctgttcggcgctggcttcggcgccgtgattaccgtggtcgtgatcgtcgtgattatcaagtgcttctgcaagcaccggtcctgcttccggcggaacgaggcctccagagagacaaacaactccctgaccttcggccccgaggaagccctggctgagcagaccgtgtttctgtgatga

SEQ ID NO: 67 - DNA, 546 bp, Artificial Sequence
  Construct GSC2819
ggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcag

SEQ ID NO: 68 - DNA, 528 bp, Artificial Sequence
  Construct GSC3469
ggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagggaggagggaacag

SEQ ID NO: 69 - DNA, 563 bp, Artificial Sequence
  Intron cTNT-I4
gtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcag

SEQ ID NO: 70 - DNA, 545 bp, Artificial Sequence
  Intron I4(0Y)
gtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagggaggagggaacag

SEQ ID NO: 71 - DNA, 700 bp, Artificial Sequence
  Intron I4(polyA)
gtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatacttgtttattgcagcttataatggttacaaataaagcaatagcatcacaaatttcacaaataaagcatttttttcactgcattctagttgtggtttgtccaaactcatcaatgtatcttatcatgtctgtatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcag

SEQ ID NO: 72 - DNA, 545 bp, Artificial Sequence
  Intron I4(3Y)
gtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagacctgaggggacag

SEQ ID NO: 73 - DNA, 545 bp, Artificial Sequence
  Intron I4(5Y)
gtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccctgtccttattgcacag

SEQ ID NO: 74 - DNA, 545 bp, Artificial Sequence
  Intron I4(5Y-5)
gtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccctgggaggattgcacag

SEQ ID NO: 75 - DNA, 545 bp, Artificial Sequence
  Intron I4(9Y)
gtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgtctccttctgggacag

SEQ ID NO: 76 - DNA, 563 bp, Artificial Sequence
  Intron I4(15Y-3)
gtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctgctacctggctcag

SEQ ID NO: 77 - DNA, 1035 bp, Artificial Sequence
  Intron I4(SV40ter)
gtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggggcaggataatatatggtagggttcatagccagagtaacctttttttttaatttttattttattttatttttgagatcagttcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcag

SEQ ID NO: 78 - DNA, 563 bp, Artificial Sequence
  Intron I4(SD_GGC)
gcaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcag

SEQ ID NO: 79 - DNA, 375 bp, Artificial Sequence
  GSB59abc
ctgtccctgggcaagtgatagaagcttgcggccgcgcttagcgatatcgaattctctagaggtacccccggggggcccctcgagttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 80 - DNA, 1094 bp, Artificial Sequence; identical to SEQ ID NO: 55
  GSC11218

SEQ ID NO: 81 - DNA, 1097 bp, Artificial Sequence
  GSC11213
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatctggctctgatcctgggacctgtgctggctctgctggcactggtggctctgggagtgctgggactgtggaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 82 - DNA, 1097 bp, Artificial Sequence
  GSC11214
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatatcgccgccatcatcgtgatcctggtgttgctcctgctgctgggcatcggcctgatgtggtggttctggaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 83 - DNA, 1094 bp, Artificial Sequence
  GSC11210
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatatggccctgattgtgctgggcggagtggctggcctgctgctgtttatcggcctgggcatcttcttcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 84 - DNA, 1094 bp, Artificial Sequence
  GSC11205
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatatcgctggcgtgatcgctggcatcctgctgttcgtgatcatcttcctgggcgtggtgctcgtgatgaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 85 - DNA, 1094 bp, Artificial Sequence
  GSC11217
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatgctctggtcgtgatccccatcatcttcggcatcctgttcgccatcctgctggtgctggtgttcatcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 86 - DNA, 1097 bp, Artificial Sequence
  GSC11756
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatatcatccctatcgtggccggcgtggtggctggcatcgtgctgattggactggccctgctgctgatctggaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 87 - DNA, 1100 bp, Artificial Sequence
  GSC11296
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatggaatcgtggctggactggctgtgctggccgtggtcgtgattggagctgtggtggccgccgtgatgtgcagaaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 88 - DNA, 1100 bp, Artificial Sequence
  GSC11295
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatctgatcattgccctgcctgtggccgtgctgctgatcgtgggaggcctcgtgatcatgctgtacgtgttccacaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 89 - DNA, 1100 bp, Artificial Sequence
  GSC11390
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatggcctgctgatcggcatctccatcgcctccctgtgcctggtggtggccctgctggccctgctgtgccacctgaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 90 - DNA, 1100 bp, Artificial Sequence
  GSC11391
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatctgctgctgggcgtgtccgtgtcctgcatcgtgatcctggccgtgtgcctgctgtgctacgtgtccatcaccaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 91 - DNA, 1097 bp, Artificial Sequence
  GSC11392
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccaacaccgtgggctccgtgatcggcgtgatcgtgaccatcttcgtgtccggcaccgtgtacttcatcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 92 - DNA, 1097 bp, Artificial Sequence
  GSC11397
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatatcaccctgatcatcttcggcgtgatggccggcgtgatcggcaccatcctgctgatctcctacggcatcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 93 - DNA, 1091 bp, Artificial Sequence
  GSC11204
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatgccctgtggctgggcgtgctgagactgctgctgttcaagctgctcctgttcgacctgctcctcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 94 - DNA, 1091 bp, Artificial Sequence
  GSC11216
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatgccctgtggctgggcgtgctggtactgctgctgttcaagctgctcctgttcgacctgctcctcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 95 - DNA, 1091 bp, Artificial Sequence
  GSC11203
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatgccctgtggctgggcgtgctgagactgctgctgttcgtgctgctcctgttcgacctgctcctcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 96 - DNA, 1091 bp, Artificial Sequence
  GSC11211
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatgccctgtggctgggcgtgctgagactgctgctgttcaagctgctcctgttcgtcctgctcctcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 97 - DNA, 1091 bp, Artificial Sequence
  GSC11212
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatgccctgtggctgggcgtgctggtactgctgctgttcgtgctgctcctgttcgacctgctcctcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 98 - DNA, 1091 bp, Artificial Sequence
  GSC11398
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatgccctgtggctgggcgtgctggtactgctgctgttcaagctgctcctgttcgtcctgctcctcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 99 - DNA, 1091 bp, Artificial Sequence
  GSC11208
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatgccctgtggctgggcgtgctgagactgctgctgttcgtgctgctcctgttcgtcctgctcctcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 100 - DNA, 1091 bp, Artificial Sequence
  GSC11291
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatgccctgtggctgggcgtgctggtactgctgctgttcgtgctgctcctgttcgtcctgctcctcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 101 - DNA, 1085 bp, Artificial Sequence
  GSC11297
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatttcttcatccccctgctggtggtgatcctgttcgccgtggacaccggcctgttcatcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 102 - DNA, 1085 bp, Artificial Sequence
  GSC11298
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatatcctgatcggcacctccgtggtgatcatcctgttcatcctgctgttcttcctgctgaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 103 - DNA, 1079 bp, Artificial Sequence
  GSC11393
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatgtgctggccctgatcgtgatcttcctgaccatcgccgtgctgctggccctgaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 104 - DNA, 1106 bp, Artificial Sequence
  GSC11394
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatgtgatgtccgtggccaccatcgtgatcgtggacatctgcatcaccggcggcctgctgctgctggtgtactactggtccaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 105 - DNA, 1106 bp, Artificial Sequence
  GSC11395
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatgccatccccatctggtgggtgctggtgggcgtgctgggcggcctgctgctgctgaccatcctggtgctggccatgtggaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 106 - DNA, 1109 bp, Artificial Sequence
  GSC11396
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatttctgggtgctggtggtggtgggcggcgtgctggcctgctactccctgctggtgaccgtggccttcatcatcttctgggtgaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 107 - DNA, 1109 bp, Artificial Sequence
  GSC11389
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatctgtggacgaccatcaccatcttcatcacactcttcctgttaagcgtgtgctacagtgccaccgtcaccttcttcaaggtgaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 108 - DNA, 1082 bp, Artificial Sequence
  GSC11677
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccaccgtgatcgtcgtgattatcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 109 - DNA, 1088 bp, Artificial Sequence
  GSC11679
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccaccgtggtcgtgatcgtcgtgattatcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 110 - DNA, 1100 bp, Artificial Sequence
  GSC11484
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccgtggtcattgtaaccgtggtcgtgatcgtcgtgattatcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 111 - DNA, 1106 bp, Artificial Sequence
  GSC11292
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccgtggtcattgtagtcgtaaccgtggtcgtgatcgtcgtgattatcaagtgcttctgcaagcaccggtcctgcttccggcggaacgaggcctccagagagacaaacaactccctgaccttcggccccgaggaagccctggctgagcagaccgtgtttctgtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 112 - DNA, 1007 bp, Artificial Sequence
  GSC11207
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccgtgattaccgtggtcgtgatcgtcgtgattatcgccagcgcccatcaccaccaccatcattgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 113 - DNA, 995 bp, Artificial Sequence
  GSC11209
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccgtgattaccgtggtcgtgatcgtcgtgattatcaagtgcttctgcaagtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 114 - DNA, 1058 bp, Artificial Sequence
  GSC11758
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccgtgattaccgtggtcgtgatcgtcgtgattatcaagtggatcttctcctcggtggtggacctgaagcagaccatcatccccgactacaggaacatgatcggacagggggcctagtgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 115 - protein, 1016 aa, Artificial Sequence
  GSC11764
CTGTCTCCGGGTAAATGATGAAAGCTTGGTAGGTGATCCTCCTGCTGCTTTGGTTCAGGGTTTTGCTTGAGGGGGGGGGGTGGTGATTTCCTTGCCATGGGCAGACTGAGCAGAAAAGGCCATTGGGACCATGTTCTGAATGCCTCCACCTCAACCACCGGCCGGTAGGACCAAAGCCACCCCGTGTTTTCTCAGGATCTCTTTTCCCAGGGAGATCCCTCGGCCCAAAGAGGGAGATGGCAATGCTGGATGTGTGCACAATAATTCAACAGGCATTGGAACTTCAGCATCGATGCTGAATGCAATTAACAATGCTCAAGCAGAACCCCCGGCTCCATCAGCACAGTGCAGGACCAAACCCCATGCTGCAGCAGTGGGGCTGTCTGTACGGGGTGGGCAATGGGAACCGGGGTCTGCTGGGGCTCCTGCTGCTTCAGTGCTGCCATGCAGCCACACATCCTGAGAGCTGAAAGGGTCGGCGTCCTCACCTGGTGCACACCGTAGCTCTGCCCCACAGCTTTAAGGCACCTGGCTAACCTCTGCGCTTCTTCCCTTCCCTCCTCCCTGGCTCAGGTGGTGGTGGATCTCCCCCCGAGGACCCCCCTGACTCCAAGAATACCCTGGTGCTGTTCGGCGCTGGCTTCGGCGCCGTGATTACCGTGGTCGTGATCGTCGTGATTATCAGGTCCCAGCAGGAGGCCGCCGCCAAGAAGTTCTTCTGATGATTCGAAATGACCGACCAAGCGACGCCCAACCTGCCATCACGAGATTTCGATTCCACCGCCGCCTTCTATGAAAGGTTGGGCTTCGGAATCGTTTTCCGGGACGCCGGCTGGATGATCCTCCAGCGCGGGGATCTCATGCTGGAGTTCTTCGCCCACCCCAACTTGTTTATTGCAGCTTATAATGGTTACAAATAAAGCAATAGCATCACAAATTTCACAAATAAAGCATTTTTTTCACTGCATTCTAGTTGTGGTTTGTCCAAACTCATCAATGTATCTTATCATGTCTGT

SEQ ID NO: 116 - DNA, 1016 bp, Artificial Sequence
  GSC11678
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccgtgattaccgtggtcgtgatcgtcgtgattatcaggtcccagcaggaggccgccgccaagaagtggtggtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 117 - DNA, 1115 bp, Artificial Sequence
  GSC11759
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccgtgattaccgtggtcgtgatcgtcgtgattatcatgttcctgaccctgaagggctccctgaagcagaggctgcaggtgatgatccagccctccgaggacatcgtgaggcccgagaacggccccgagcagccccaggccggctcctccgcctccaaggaggcctacatctgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 118 - DNA, 1004 bp, Artificial Sequence
  GSC11483
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccgtgattaccgtggtcgtgatcgtcgtgattatcatgttcctgaccctgaagggctcctgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 119 - DNA, 1115 bp, Artificial Sequence
  GSC11760
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccgtgattaccgtggtcgtgatcgtcgtgattatctgcttcgtgggcaacaggttccagcagaagctgaggtccgtgttcagggtgcccatcacctggctgcagggcaagagggagaccatgtcctgcaggaagtcctcctccctgagggagatggacaccttcgtgtcctgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 120 - DNA, 1094 bp, Artificial Sequence
  GSC11761
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccgtgattaccgtggtcgtgatcgtcgtgattatctgcttcgtgggcaacaggttccagcagaagctgaggtccgtgttcagggtgcccatcacctggctgcagggcaagagggagaccatgtcctgcaggaagtcctcctccctgaggtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 121 - DNA, 1229 bp, Artificial Sequence
  GSC11762
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccgtgattaccgtggtcgtgatcgtcgtgattatcaactcccacctgctgcccaggcccctgaggcacctggcctgctgcggcggcccccagcccaggatgaggaggaggctgtccgacggctccctgtcctccaggcacaccaccctgctgaccaggtcctcctgccccgccaccctgtccctgtccctgtccctgaccctgtccggcaggcccaggcccgaggagtcccccagggacctggagctggccgacggcgagggcaccgccgagaccatcatcttctgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 122 - DNA, 1052 bp, Artificial Sequence
  GSC11765
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccgtgattaccgtggtcgtgatcgtcgtgattatcaactcccacgaggagtcccccagggacctggagctggccgacggcgagggcaccgccgagaccatcatcttctgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 123 - DNA, 1049 bp, Artificial Sequence
  GSC11766
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccgtgattaccgtggtcgtgatcgtcgtgattatctgcatcaagctgaagcacaccaagaagaggcagatctacaccgacatcgagatgaacaggctgggcaagtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 124 - DNA, 1049 bp, Artificial Sequence
  GSC11763
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccgtgattaccgtggtcgtgatcgtcgtgattatctgcatcaagctgaagcacaccaagaagaggcagatcgccgccgccgccgccgccaacaggctgggcaagtgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 125 - DNA, 1118 bp, Artificial Sequence
  GSC11294
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcagagctgcaaccggtggagagctgtgcggaggcgcaggacggggagctggacgggctgtggacgaccatcaccatcttcatcacactcttcctgttaagcgtgtgctacagtgccaccgtcaccttcttcaaggtgaagtgcttctgcaagcaccggtcctgcttccggcggaacgaggcctccagagagacaaacaactccctgaccttcggccccgaggaagccctggctgagcagaccgtgtttctgtagtgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 126 - protein, 466 aa, Artificial Sequence
  GSB59abc
MESPAQLLFLLLLWLPDGVHAQVTLKESGPALVKPTQTLTLTCSFSGFSLSTSGMGVGWIRQPPGKALEWIAHIWWDDDKYYNTALKTRLTISKDTSKNQVVLTMTNMDPVDTATYYCARIDWDGFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 127 - protein, 80 aa, Artificial Sequence
  GSC11218
LSLSPGGGGSPPEDPPDSKNTLVLFGAGFGAVITVVVIVVIIKCFCKHRSCFRRNEASRETNNSLTFGPEEALAEQTVFL

SEQ ID NO: 128 - protein, 81 aa, Artificial Sequence
  GSC11213
LSLSPGGGGSPPEDPPDSKNLALILGPVLALLALVALGVLGLWKCFCKHRSCFRRNEASRETNNSLTFGPEEALAEQTVFL

SEQ ID NO: 129 - protein, 81 aa, Artificial Sequence
  GSC11214
LSLSPGGGGSPPEDPPDSKNIAAIIVILVLLLLLGIGLMWWFWKCFCKHRSCFRRNEASRETNNSLTFGPEEALAEQTVFL

SEQ ID NO: 130 - protein, 80 aa, Artificial Sequence
  GSC11210
LSLSPGGGGSPPEDPPDSKNMALIVLGGVAGLLLFIGLGIFFKCFCKHRSCFRRNEASRETNNSLTFGPEEALAEQTVFL

SEQ ID NO: 131 - protein, 80 aa, Artificial Sequence
  GSC11205
LSLSPGGGGSPPEDPPDSKNIAGVIAGILLFVIIFLGVVLVMKCFCKHRSCFRRNEASRETNNSLTFGPEEALAEQTVFL

SEQ ID NO: 132 - protein, 80 aa, Artificial Sequence
  GSC11217
LSLSPGGGGSPPEDPPDSKNALVVIPIIFGILFAILLVLVFIKCFCKHRSCFRRNEASRETNNSLTFGPEEALAEQTVFL

SEQ ID NO: 133 - protein, 81 aa, Artificial Sequence
  GSC11756
LSLSPGGGGSPPEDPPDSKNIIPIVAGVVAGIVLIGLALLLIWKCFCKHRSCFRRNEASRETNNSLTFGPEEALAEQTVFL

SEQ ID NO: 134 - protein, 82 aa, Artificial Sequence
  GSC11296
LSLSPGGGGSPPEDPPDSKNGIVAGLAVLAVVVIGAVVAAVMCRKCFCKHRSCFRRNEASRETNNSLTFGPEEALAEQTVFL

SEQ ID NO: 135 - protein, 82 aa, Artificial Sequence
  GSC11295
LSLSPGGGGSPPEDPPDSKNLIIALPVAVLLIVGGLVIMLYVFHKCFCKHRSCFRRNEASRETNNSLTFGPEEALAEQTVFL

SEQ ID NO: 136 - protein, 82 aa, Artificial Sequence
  GSC11390
LSLSPGGGGSPPEDPPDSKNGLLIGISIASLCLVVALLALLCHLKCFCKHRSCFRRNEASRETNNSLTFGPEEALAEQTVFL

SEQ ID NO: 137 - protein, 82 aa, Artificial Sequence
  GSC11391
LSLSPGGGGSPPEDPPDSKNLLLGVSVSCIVILAVCLLCYVSITKCFCKHRSCFRRNEASRETNNSLTFGPEEALAEQTVFL

SEQ ID NO: 138 - protein, 81 aa, Artificial Sequence
  GSC11392
LSLSPGGGGSPPEDPPDSKNTNTVGSVIGVIVTIFVSGTVYFIKCFCKHRSCFRRNEASRETNNSLTFGPEEALAEQTVFL

SEQ ID NO: 139 - protein, 81 aa, Artificial Sequence
  GSC11397
LSLSPGGGGSPPEDPPDSKNITLIIFGVMAGVIGTILLISYGIKCFCKHRSCFRRNEASRETNNSLTFGPEEALAEQTVFL

SEQ ID NO: 140 - protein, 79 aa, Artificial Sequence
  GSC11204
LSLSPGGGGSPPEDPPDSKNALWLGVLRLLLFKLLLFDLLLKCFCKHRSCFRRNEASRETNNSLTFGPEEALAEQTVFL

SEQ ID NO: 141 - protein, 79 aa, Artificial Sequence
  GSC11216
LSLSPGGGGSPPEDPPDSKNALWLGVLVLLLFKLLLFDLLLKCFCKHRSCFRRNEASRETNNSLTFGPEEALAEQTVFL

SEQ ID NO: 142 - protein, 79 aa, Artificial Sequence
  GSC11203
LSLSPGGGGSPPEDPPDSKNALWLGVLRLLLFVLLLFDLLLKCFCKHRSCFRRNEASRETNNSLTFGPEEALAEQTVFL

SEQ ID NO: 143 - protein, 79 aa, Artificial Sequence
  GSC11211
LSLSPGGGGSPPEDPPDSKNALWLGVLRLLLFKLLLFVLLLKCFCKHRSCFRRNEASRETNNSLTFGPEEALAEQTVFL

SEQ ID NO: 144 - protein, 79 aa, Artificial Sequence
  GSC11212
LSLSPGGGGSPPEDPPDSKNALWLGVLVLLLFVLLLFDLLLKCFCKHRSCFRRNEASRETNNSLTFGPEEALAEQTVFL

SEQ ID NO: 145 - protein, 79 aa, Artificial Sequence
  GSC11398
LSLSPGGGGSPPEDPPDSKNALWLGVLVLLLFKLLLFVLLLKCFCKHRSCFRRNEASRETNNSLTFGPEEALAEQTVFL

SEQ ID NO: 146 - protein, 79 aa, Artificial Sequence
  GSC11208
LSLSPGGGGSPPEDPPDSKNALWLGVLRLLLFVLLLFVLLLKCFCKHRSCFRRNEASRETNNSLTFGPEEALAEQTVFL

SEQ ID NO: 147 - protein, 79 aa, Artificial Sequence
  GSC11291
LSLSPGGGGSPPEDPPDSKNALWLGVLVLLLFVLLLFVLLLKCFCKHRSCFRRNEASRETNNSLTFGPEEALAEQTVFL

SEQ ID NO: 148 - protein, 77 aa, Artificial Sequence
  GSC11297
LSLSPGGGGSPPEDPPDSKNFFIPLLVVILFAVDTGLFIKCFCKHRSCFRRNEASRETNNSLTFGPEEALAEQTVFL

SEQ ID NO: 149 - protein, 77 aa, Artificial Sequence
  GSC11298
LSLSPGGGGSPPEDPPDSKNILIGTSVVIILFILLFFLLKCFCKHRSCFRRNEASRETNNSLTFGPEEALAEQTVFL

SEQ ID NO: 150 - protein, 75 aa, Artificial Sequence
  GSC11393
LSLSPGGGGSPPEDPPDSKNVLALIVIFLTIAVLLALKCFCKHRSCFRRNEASRETNNSLTFGPEEALAEQTVFL

SEQ ID NO: 151 - protein, 84 aa, Artificial Sequence
  GSC11394
LSLSPGGGGSPPEDPPDSKNVMSVATIVIVDICITGGLLLLVYYWSKCFCKHRSCFRRNEASRETNNSLTFGPEEALAEQTVFL

SEQ ID NO: 152 - protein, 84 aa, Artificial Sequence
  GSC11395
LSLSPGGGGSPPEDPPDSKNAIPIWWVLVGVLGGLLLLTILVLAMWKCFCKHRSCFRRNEASRETNNSLTFGPEEALAEQTVFL

SEQ ID NO: 153 - protein, 85 aa, Artificial Sequence
  GSC11396
LSLSPGGGGSPPEDPPDSKNFWVLVVVGGVLACYSLLVTVAFIIFWVKCFCKHRSCFRRNEASRETNNSLTFGPEEALAEQTVFL

SEQ ID NO: 154 - protein, 85 aa, Artificial Sequence
  GSC11389
LSLSPGGGGSPPEDPPDSKNLWTTITIFITLFLLSVCYSATVTFFKVKCFCKHRSCFRRNEASRETNNSLTFGPEEALAEQTVFL

SEQ ID NO: 155 - protein, 76 aa, Artificial Sequence
  GSC11677
LSLSPGGGGSPPEDPPDSKNTLVLFGAGFGATVIVVIIKCFCKHRSCFRRNEASRETNNSLTFGPEEALAEQTVFL

SEQ ID NO: 156 - protein, 78 aa, Artificial Sequence
  GSC11679
LSLSPGGGGSPPEDPPDSKNTLVLFGAGFGATVVVIVVIIKCFCKHRSCFRRNEASRETNNSLTFGPEEALAEQTVFL

SEQ ID NO: 157 - protein, 82 aa, Artificial Sequence
  GSC11484
LSLSPGGGGSPPEDPPDSKNTLVLFGAGFGAVVIVTVVVIVVIIKCFCKHRSCFRRNEASRETNNSLTFGPEEALAEQTVFL

SEQ ID NO: 158 - protein, 84 aa, Artificial Sequence
  GSC11292
LSLSPGGGGSPPEDPPDSKNTLVLFGAGFGAVVIVVVTVVVIVVIIKCFCKHRSCFRRNEASRETNNSLTFGPEEALAEQTVFL

SEQ ID NO: 159 - protein, 51 aa, Artificial Sequence
  GSC11207
LSLSPGGGGSPPEDPPDSKNTLVLFGAGFGAVITVVVIVVIIASAHHHHHH

SEQ ID NO: 160 - protein, 47 aa, Artificial Sequence
  GSC11209
LSLSPGGGGSPPEDPPDSKNTLVLFGAGFGAVITVVVIVVIIKCFCK

SEQ ID NO: 161 - protein, 68 aa, Artificial Sequence
  GSC11758
LSLSPGGGGSPPEDPPDSKNTLVLFGAGFGAVITVVVIVVIIKWIFSSVVDLKQTIIPDYRNMIGQGA

SEQ ID NO: 162 - protein, 54 aa, Artificial Sequence
  GSC11764
LSLSPGGGGSPPEDPPDSKNTLVLFGAGFGAVITVVVIVVIIRSQQEAAAKKFF

SEQ ID NO: 163 - protein, 54 aa, Artificial Sequence
  GSC11678
LSLSPGGGGSPPEDPPDSKNTLVLFGAGFGAVITVVVIVVIIRSQQEAAAKKWW

SEQ ID NO: 164 - protein, 87 aa, Artificial Sequence
  GSC11759
LSLSPGGGGSPPEDPPDSKNTLVLFGAGFGAVITVVVIVVIIMFLTLKGSLKQRLQVMIQPSEDIVRPENGPEQPQAGSSASKEAYI

SEQ ID NO: 165 - protein, 50 aa, Artificial Sequence
  GSC11483
LSLSPGGGGSPPEDPPDSKNTLVLFGAGFGAVITVVVIVVIIMFLTLKGS

SEQ ID NO: 166 - protein, 87 aa, Artificial Sequence
  GSC11760
LSLSPGGGGSPPEDPPDSKNTLVLFGAGFGAVITVVVIVVIICFVGNRFQQKLRSVFRVPITWLQGKRETMSCRKSSSLREMDTFVS

SEQ ID NO: 167 - protein, 80 aa, Artificial Sequence
  GSC11761
LSLSPGGGGSPPEDPPDSKNTLVLFGAGFGAVITVVVIVVIICFVGNRFQQKLRSVFRVPITWLQGKRETMSCRKSSSLR

SEQ ID NO: 168 - protein, 125 aa, Artificial Sequence
  GSC11762
LSLSPGGGGSPPEDPPDSKNTLVLFGAGFGAVITVVVIVVIINSHLLPRPLRHLACCGGPQPRMRRRLSDGSLSSRHTTLLTRSSCPATLSLSLSLTLSGRPRPEESPRDLELADGEGTAETIIF

SEQ ID NO: 169 - protein, 66 aa, Artificial Sequence
  GSC11765
LSLSPGGGGSPPEDPPDSKNTLVLFGAGFGAVITVVVIVVIINSHEESPRDLELADGEGTAETIIF

SEQ ID NO: 170 - protein, 65 aa, Artificial Sequence
  GSC11766
LSLSPGGGGSPPEDPPDSKNTLVLFGAGFGAVITVVVIVVIICIKLKHTKKRQIYTDIEMNRLGK

SEQ ID NO: 171 - protein, 65 aa, Artificial Sequence
  GSC11763
LSLSPGGGGSPPEDPPDSKNTLVLFGAGFGAVITVVVIVVIICIKLKHTKKRQIAAAAAANRLGK

SEQ ID NO: 172 - protein, 88 aa, Artificial Sequence
  GSC11294
LSLSPELQPVESCAEAQDGELDGLWTTITIFITLFLLSVCYSATVTFFKVKCFCKHRSCFRRNEASRETNNSLTFGPEEALAEQTVFL

SEQ ID NO: 173 - protein, 22 aa, Artificial Sequence
  B7 TM domain
TLVLFGAGFGAVITVVVIVVII

SEQ ID NO: 174 - protein, 23 aa, Homo sapiens
LALILGPVLALLALVALGVLGLW

SEQ ID NO: 175 - protein, 23 aa, Homo sapiens
IAAIIVILVLLLLLGIGLMWWFW

SEQ ID NO: 176 - protein, 22 aa, Homo sapiens
MALIVLGGVAGLLLFIGLGIFF

SEQ ID NO: 177 - protein, 22 aa, Homo sapiens
IAGVIAGILLFVIIFLGVVLVM

SEQ ID NO: 178 - protein, 22 aa, Homo sapiens
ALVVIPIIFGILFAILLVLVFI

SEQ ID NO: 179 - protein, 23 aa, Homo sapiens
IIPIVAGVVAGIVLIGLALLLIW

SEQ ID NO: 180 - protein, 24 aa, Homo sapiens
GIVAGLAVLAVVVIGAVVAAVMCR

SEQ ID NO: 181 - protein, 24 aa, Homo sapiens
LIIALPVAVLLIVGGLVIMLYVFH

SEQ ID NO: 182 - protein, 24 aa, Homo sapiens
GLLIGISIASLCLVVALLALLCHL

SEQ ID NO: 183 - protein, 24 aa, Homo sapiens
LLLGVSVSCIVILAVCLLCYVSIT

SEQ ID NO: 184 - protein, 23 aa, Homo sapiens
TNTVGSVIGVIVTIFVSGTVYFI

SEQ ID NO: 185 - protein, 23 aa, Homo sapiens
ITLIIFGVMAGVIGTILLISYGI

SEQ ID NO: 186 - protein, 21 aa, Artificial Sequence
  PTCRA TM domain
ALWLGVLRLLLFKLLLFDLLL

SEQ ID NO: 187 - protein, 21 aa, Artificial Sequence
  PTCRA_R8V TM domain
ALWLGVLVLLLFKLLLFDLLL

SEQ ID NO: 188 - protein, 21 aa, Artificial Sequence
  PTCRA_K13V TM domain
ALWLGVLRLLLFVLLLFDLLL

SEQ ID NO: 189 - protein, 21 aa, Artificial Sequence
  PTCRA_D18V TM domain
ALWLGVLRLLLFKLLLFVLLL

SEQ ID NO: 190 - protein, 21 aa, Artificial Sequence
  PTCRA_R8V_K13V TM domain
ALWLGVLVLLLFVLLLFDLLL

SEQ ID NO: 191 - protein, 21 aa, Artificial Sequence
  PTCRA_R8V_D18V TM domain
ALWLGVLVLLLFKLLLFVLLL

SEQ ID NO: 192 - protein, 21 aa, Artificial Sequence
  PTCRA_K13V_D18V TM domain
ALWLGVLRLLLFVLLLFVLLL

SEQ ID NO: 193 - protein, 21 aa, Artificial Sequence
  PTCRA_R8V_K13V_D18V TM domain
ALWLGVLVLLLFVLLLFVLLL

SEQ ID NO: 194 - protein, 19 aa, Homo sapiens
FFIPLLVVILFAVDTGLFI

SEQ ID NO: 195 - protein, 19 aa, Homo sapiens
ILIGTSVVIILFILLFFLL

SEQ ID NO: 196 - protein, 17 aa, Homo sapiens
VLALIVIFLTIAVLLAL

SEQ ID NO: 197 - protein, 26 aa, Homo sapiens
VMSVATIVIVDICITGGLLLLVYYWS

SEQ ID NO: 198 - protein, 26 aa, Homo sapiens
AIPIWWVLVGVLGGLLLLTILVLAMW

SEQ ID NO: 199 - protein, 27 aa, Homo sapiens
FWVLVVVGGVLACYSLLVTVAFIIFWV

SEQ ID NO: 200 - protein, 27 aa, Artificial Sequence
  M1M2 TM domain
LWTTITIFITLFLLSVCYSATVTFFKV

SEQ ID NO: 201 - protein, 18 aa, Artificial Sequence
  B7(18) TM domain
TLVLFGAGFGATVIVVII

SEQ ID NO: 202 - protein, 20 aa, Artificial Sequence
  B7(20) TM domain
TLVLFGAGFGATVVVIVVII

SEQ ID NO: 203 - protein, 24 aa, Artificial Sequence
  B7(24) TM domain
TLVLFGAGFGAVVIVTVVVIVVII

SEQ ID NO: 204 - protein, 26 aa, Artificial Sequence
  B7(26) TM domain
TLVLFGAGFGAVVIVVVTVVVIVVII

SEQ ID NO: 205 - protein, 9 aa, Artificial Sequence
  6His cyto tail
ASAHHHHHH

SEQ ID NO: 206 - protein, 5 aa, Artificial Sequence
  KCFCK cyto tail
KCFCK

SEQ ID NO: 207 - protein, 26 aa, Artificial Sequence
  M1M2 cyto tail
KWIFSSVVDLKQTIIPDYRNMIGQGA

SEQ ID NO: 208 - protein, 12 aa, Artificial Sequence
  ERGIC53 cyto tail
RSQQEAAAKKFF

SEQ ID NO: 209 - protein, 12 aa, Artificial Sequence
  ERGICnoER cyto tail
RSQQEAAAKKWW

SEQ ID NO: 210 - protein, 45 aa, Artificial Sequence
  GAT1 cyto tail
MFLTLKGSLKQRLQVMIQPSEDIVRPENGPEQPQAGSSASKEAYI

SEQ ID NO: 211 - protein, 8 aa, Artificial Sequence
  GAT1noER cyto tail
MFLTLKGS

SEQ ID NO: 212 - protein, 45 aa, Artificial Sequence
  AGTR2 cyto tail
CFVGNRFQQKLRSVFRVPITWLQGKRETMSCRKSSSLREMDTFVS

SEQ ID NO: 213 - protein, 38 aa, Artificial Sequence
  AGTR2noER cyto tail
CFVGNRFQQKLRSVFRVPITWLQGKRETMSCRKSSSLR

SEQ ID NO: 214 - protein, 83 aa, Artificial Sequence
  V1BR cyto tail
NSHLLPRPLRHLACCGGPQPRMRRRLSDGSLSSRHTTLLTRSSCPATLSLSLSLTLSGRPRPEESPRDLELADGEGTAETIIF

SEQ ID NO: 215 - protein, 24 aa, Artificial Sequence
  V1BRnoER cyto tail
NSHEESPRDLELADGEGTAETIIF

SEQ ID NO: 216 - protein, 23 aa, Artificial Sequence
  VGLG cyto tail
CIKLKHTKKRQIYTDIEMNRLGK

SEQ ID NO: 217 - protein, 23 aa, Artificial Sequence
  VGLGnoER cyto tail
CIKLKHTKKRQIAAAAAANRLGK

SEQ ID NO: 218 - protein, 38 aa, Artificial Sequence
  B7 cyto tail
KCFCKHRSCFRRNEASRETNNSLTFGPEEALAEQTVFL

SEQ ID NO: 219 - DNA, 39 bp, Artificial Sequence
  Primer GlnPr2376
ggccgttcgaatcatcacttgcagaagcacttgataatc

SEQ ID NO: 220 - DNA, 65 bp, Artificial Sequence
  Primer GlnPr2377
gtcatttcgaatcatcaatgatggtggtggtgatgggcgctggcgataatcacgacgatcacgac

SEQ ID NO: 221 - protein, 59 aa, Artificial Sequence
  Primer GlnPr2378
TTGAACAGCAGCAGTCTCAGCACGCCCAGCCACAGGGCCCCGTCCAGCTCCCCGTCCTG

SEQ ID NO: 222 - DNA, 66 bp, Artificial Sequence
  Primer GlnPr2379
ctgagactgctgctgttcaagctgctcctgttcgacctgctcctcaagtggatcttctcctcggtg

SEQ ID NO: 223 - DNA, 64 bp, Artificial Sequence
  Primer GlnPr2380
gcttgaacagcagcagtctcagcacgcccagccacagggcattcttggagtcaggggggtcctc

SEQ ID NO: 224 - protein, 69 aa, Artificial Sequence
  Primer GlnPr2381
CAGGACCGGTGCTTGCAGAAGCACTTGAGGAGCAGGTCGAACAGGAGCAGCTTGAACAGCAGCAGTCTC

SEQ ID NO: 225 - DNA, 64 bp, Artificial Sequence
  Primer GlnPr2382
gcttgaacagcagcagtaccagcacgcccagccacagggcattcttggagtcaggggggtcctc

SEQ ID NO: 226 - DNA, 69 bp, Artificial Sequence
  Primer GlnPr2383
caggaccggtgcttgcagaagcacttgaggagcaggtcgaacaggagcagcttgaacagcagcagtacc

SEQ ID NO: 227 - DNA, 66 bp, Artificial Sequence
  Primer GlnPr2384
gaccggtgcttgcagaagcacttgaggagcaggtcgaacaggagcagcacgaacagcagcagtctc

SEQ ID NO: 228 - DNA, 66 bp, Artificial Sequence
  Primer GlnPr2385
gaccggtgcttgcagaagcacttgaggagcaggacgaacaggagcagcttgaacagcagcagtctc

SEQ ID NO: 229 - DNA, 69 bp, Artificial Sequence
  Primer GlnPr2386
caggaccggtgcttgcagaagcacttgaggagcaggtcgaacaggagcagcacgaacagcagcagtacc

SEQ ID NO: 230 - DNA, 69 bp, Artificial Sequence
  Primer GlnPr2387
caggaccggtgcttgcagaagcacttgaggagcaggacgaacaggagcagcttgaacagcagcagtacc

SEQ ID NO: 231 - DNA, 66 bp, Artificial Sequence
  Primer GlnPr2388
gaccggtgcttgcagaagcacttgaggagcaggacgaacaggagcagcacgaacagcagcagtctc

SEQ ID NO: 232 - DNA, 69 bp, Artificial Sequence
  Primer GlnPr2389
caggaccggtgcttgcagaagcacttgaggagcaggacgaacaggagcagcacgaacagcagcagtacc

SEQ ID NO: 233 - DNA, 47 bp, Artificial Sequence
  Primer GlnPr2390
ggtcgtgatcgtcgtgattatcaagtggatcttctcctcggtggtgg

SEQ ID NO: 234 - DNA, 47 bp, Artificial Sequence
  Primer GlnPr2391
ccaccaccgaggagaagatccacttgataatcacgacgatcacgacc

SEQ ID NO: 235 - DNA, 42 bp, Artificial Sequence
  Primer GlnPr2392
cttcggcgccaccgtggtcgtgatcgtcgtgattatcaagtg

SEQ ID NO: 236 - DNA, 42 bp, Artificial Sequence
  Primer GlnPr2393
caggaccggtgcttgcagaagcacttgataatcacgacgatc

SEQ ID NO: 237 - DNA, 40 bp, Artificial Sequence
  Primer GlnPr2394
cttcggcgccaccgtgatcgtcgtgattatcaagtgcttc

SEQ ID NO: 238 - DNA, 38 bp, Artificial Sequence
  Primer GlnPr2395
caggaccggtgcttgcagaagcacttgataatcacgac

SEQ ID NO: 239 - DNA, 43 bp, Artificial Sequence
  Primer GlnPr2396
cttcggcgccgtggtcattgtaaccgtggtcgtgatcgtcgtg

SEQ ID NO: 240 - DNA, 50 bp, Artificial Sequence
  Primer GlnPr2397
caggaccggtgcttgcagaagcacttgataatcacgacgatcacgaccac

SEQ ID NO: 241 - DNA, 49 bp, Artificial Sequence
  Primer GlnPr2398
cttcggcgccgtggtcattgtagtcgtaaccgtggtcgtgatcgtcgtg

SEQ ID NO: 242 - DNA, 52 bp, Artificial Sequence
  Primer GlnPr2399
caggaccggtgcttgcagaagcacttgataatcacgacgatcacgaccacgg

SEQ ID NO: 243 - DNA, 70 bp, Artificial Sequence
  Primer GlnPr2400
gagccaccagtgccagcagagccagcacaggtcccaggatcagagccagattcttggagtcaggggggtc

SEQ ID NO: 244 - DNA, 65 bp, Artificial Sequence
  > Seq Id No.244Primer GlnPr2401
caggaccggtgcttgcagaagcacttccacagtcccagcactcccagagccaccagtgccagcag

SEQ ID NO: 245 - DNA, 70 bp, Artificial Sequence
  Primer GlnPr2402
cgatgcccagcagcaggagcaacaccaggatcacgatgatggcggcgatattcttggagtcaggggggtc

SEQ ID NO: 246 - DNA, 65 bp, Artificial Sequence
  Primer GlnPr2403
caggaccggtgcttgcagaagcacttccagaaccaccacatcaggccgatgcccagcagcaggag

SEQ ID NO: 247 - DNA, 69 bp, Artificial Sequence
  Primer GlnPr2404
gataaacagcagcaggccagccactccgcccagcacaatcagggccatattcttggagtcaggggggtc

SEQ ID NO: 248 - DNA, 62 bp, Artificial Sequence
  Primer GlnPr2405
caggaccggtgcttgcagaagcacttgaagaagatgcccaggccgataaacagcagcaggcc

SEQ ID NO: 249 - DNA, 70 bp, Artificial Sequence
  Primer GlnPr2406
gtccaatcagcacgatgccagccaccacgccggccacgatagggatgatattcttggagtcaggggggtc

SEQ ID NO: 250 - DNA, 65 bp, Artificial Sequence
  Primer GlnPr2407
caggaccggtgcttgcagaagcacttccagatcagcagcagggccagtccaatcagcacgatgcc

SEQ ID NO: 251 - DNA, 70 bp, Artificial Sequence
  Primer GlnPr2408
ccaggaagatgatcacgaacagcaggatgccagcgatcacgccagcgatattcttggagtcaggggggtc

SEQ ID NO: 252 - DNA, 62 bp, Artificial Sequence
  Primer GlnPr2409
caggaccggtgcttgcagaagcacttcatcacgagcaccacgcccaggaagatgatcacgaa

SEQ ID NO: 253 - DNA, 70 bp, Artificial Sequence
  Primer GlnPr2410
gcaggatggcgaacaggatgccgaagatgatggggatcacgaccagagcattcttggagtcaggggggtc

SEQ ID NO: 254 - DNA, 63 bp, Artificial Sequence
  Primer GlnPr2411
caggaccggtgcttgcagaagcacttgatgaacaccagcaccagcaggatggcgaacaggatg

SEQ ID NO: 255 - DNA, 65 bp, Artificial Sequence
  Primer GlnPr2412
cttcggcgccgtgattaccgtggtcgtgatcgtcgtgattatcaggtcccagcaggaggccgccg

SEQ ID NO: 256 - DNA, 51 bp, Artificial Sequence
  Primer GlnPr2413
gtcatttcgaatcatcagaagaacttcttggcggcggcctcctgctgggac

SEQ ID NO: 257 - DNA, 51 bp, Artificial Sequence
  Primer GlnPr2414
gtcatttcgaatcatcaccaccacttcttggcggcggcctcctgctgggac

SEQ ID NO: 258 - DNA, 63 bp, Artificial Sequence
  Primer GlnPr2415
ggtcatttcgaatcatcaggagcccttcagggtcaggaacatgataatcacgacgatcacgac

SEQ ID NO: 259 - DNA, 39 bp, Artificial Sequence
  Primer GlnPr1285
cggaagaattcagccacagctttaaggcacctggctaac

SEQ ID NO: 260 - DNA, 66 bp, Artificial Sequence
  Primer GlnPr1494
acgaactagtccaccatggaaagcccagcccagctgctgttcctgctgctgctgtggctgcccgac

SEQ ID NO: 261 - DNA, 72 bp, Artificial Sequence
  Primer GlnPr2294
ctaccaagctttcatcatttacccggagacagggagaggctcttctgcgtgtagtggttgtgcaaggcctcg

SEQ ID NO: 262 - DNA, 432 bp, Artificial Sequence
  GeneArt_Seq80
atcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatctgtggacgaccatcaccatcttcatcacactcttcctgttaagcgtgtgctacagtgccaccgtcaccttcttcaaggtgaagtgcttctgcaagcaccggt

SEQ ID NO: 263 - DNA, 500 bp, Artificial Sequence
  GeneArt_Seq81
atcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatctgtggacgaccatcaccatcttcatcacactcttcctgttaagcgtgtgctacagtgccaccgtcaccttcttcaaggtgaagtggatcttctcctcggtggtggacctgaagcagaccatcatccccgactacaggaacatgatcggacagggggcctagtgattcgaa

SEQ ID NO: 264 - DNA, 545 bp, Artificial Sequence
  GeneArt_Seq82
atcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcagagctgcaaccggtggagagctgtgcggaggcgcaggacggggagctggacgggctgtggacgaccatcaccatcttcatcacactcttcctgttaagcgtgtgctacagtgccaccgtcaccttcttcaaggtgaagtgcttctgcaagcaccggtcctgcttccggcggaacgaggcctccagagagacaaacaactccctgaccttcggccccgaggaagccctggctgagcagaccgtgtttctgtagtgattcgaa

SEQ ID NO: 265 - DNA, 423 bp, Artificial Sequence
  GeneArt_Seq83
atcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatctgatcattgccctgcctgtggccgtgctgctgatcgtgggaggcctcgtgatcatgctgtacgtgttccacaagtgcttctgcaagcaccggt

SEQ ID NO: 266 - DNA, 423 bp, Artificial Sequence
  GeneArt_Seq84
atcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatggaatcgtggctggactggctgtgctggccgtggtcgtgattggagctgtggtggccgccgtgatgtgcagaaagtgcttctgcaagcaccggt

SEQ ID NO: 267 - DNA, 423 bp, Artificial Sequence
  GeneArt_Seq85
atcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatggcctgctgatcggcatctccatcgcctccctgtgcctggtggtggccctgctggccctgctgtgccacctgaagtgcttctgcaagcaccggt

SEQ ID NO: 268 - DNA, 423 bp, Artificial Sequence
  GeneArt_Seq86
atcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatctgctgctgggcgtgtccgtgtcctgcatcgtgatcctggccgtgtgcctgctgtgctacgtgtccatcaccaagtgcttctgcaagcaccggt

SEQ ID NO: 269 - DNA, 420 bp, Artificial Sequence
  GeneArt_Seq87
atcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccaacaccgtgggctccgtgatcggcgtgatcgtgaccatcttcgtgtccggcaccgtgtacttcatcaagtgcttctgcaagcaccggt

SEQ ID NO: 270 - DNA, 402 bp, Artificial Sequence
  GeneArt_Seq88
atcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatgtgctggccctgatcgtgatcttcctgaccatcgccgtgctgctggccctgaagtgcttctgcaagcaccggt

SEQ ID NO: 271 - DNA, 408 bp, Artificial Sequence
  GeneArt_Seq89
atcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatttcttcatccccctgctggtggtgatcctgttcgccgtggacaccggcctgttcatcaagtgcttctgcaagcaccggt

SEQ ID NO: 272 - DNA, 408 bp, Artificial Sequence
  GeneArt_Seq90
atcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatatcctgatcggcacctccgtggtgatcatcctgttcatcctgctgttcttcctgctgaagtgcttctgcaagcaccggt

SEQ ID NO: 273 - DNA, 429 bp, Artificial Sequence
  GeneArt_Seq91
atcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatgtgatgtccgtggccaccatcgtgatcgtggacatctgcatcaccggcggcctgctgctgctggtgtactactggtccaagtgcttctgcaagcaccggt

SEQ ID NO: 274 - DNA, 429 bp, Artificial Sequence
  GeneArt_Seq92
atcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatgccatccccatctggtgggtgctggtgggcgtgctgggcggcctgctgctgctgaccatcctggtgctggccatgtggaagtgcttctgcaagcaccggt

SEQ ID NO: 275 - DNA, 432 bp, Artificial Sequence
  GeneArt_Seq93
atcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatttctgggtgctggtggtggtgggcggcgtgctggcctgctactccctgctggtgaccgtggccttcatcatcttctgggtgaagtgcttctgcaagcaccggt

SEQ ID NO: 276 - DNA, 420 bp, Artificial Sequence
  GeneArt_Seq94
atcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaatatcaccctgatcatcttcggcgtgatggccggcgtgatcggcaccatcctgctgatctcctacggcatcaagtgcttctgcaagcaccggt

SEQ ID NO: 277 - DNA, 186 bp, Artificial Sequence
  GeneArt_Seq95
ggcgccgtgattaccgtggtcgtgatcgtcgtgattatcatgttcctgaccctgaagggctccctgaagcagaggctgcaggtgatgatccagccctccgaggacatcgtgaggcccgagaacggccccgagcagccccaggccggctcctccgcctccaaggaggcctacatctgatgattcgaa

SEQ ID NO: 278 - DNA, 186 bp, Artificial Sequence
  GeneArt_Seq96
ggcgccgtgattaccgtggtcgtgatcgtcgtgattatctgcttcgtgggcaacaggttccagcagaagctgaggtccgtgttcagggtgcccatcacctggctgcagggcaagagggagaccatgtcctgcaggaagtcctcctccctgagggagatggacaccttcgtgtcctgatgattcgaa

SEQ ID NO: 279 - DNA, 165 bp, Artificial Sequence
  GeneArt_Seq97
ggcgccgtgattaccgtggtcgtgatcgtcgtgattatctgcttcgtgggcaacaggttccagcagaagctgaggtccgtgttcagggtgcccatcacctggctgcagggcaagagggagaccatgtcctgcaggaagtcctcctccctgaggtgatgattcgaa

SEQ ID NO: 280 - DNA, 300 bp, Artificial Sequence
  GeneArt_Seq98
ggcgccgtgattaccgtggtcgtgatcgtcgtgattatcaactcccacctgctgcccaggcccctgaggcacctggcctgctgcggcggcccccagcccaggatgaggaggaggctgtccgacggctccctgtcctccaggcacaccaccctgctgaccaggtcctcctgccccgccaccctgtccctgtccctgtccctgaccctgtccggcaggcccaggcccgaggagtcccccagggacctggagctggccgacggcgagggcaccgccgagaccatcatcttctgatgattcgaa

SEQ ID NO: 281 - DNA, 123 bp, Artificial Sequence
  GeneArt_Seq99
ggcgccgtgattaccgtggtcgtgatcgtcgtgattatcaactcccacgaggagtcccccagggacctggagctggccgacggcgagggcaccgccgagaccatcatcttctgatgattcgaa

SEQ ID NO: 282 - DNA, 120 bp, Artificial Sequence
  GeneArt_Seq100
ggcgccgtgattaccgtggtcgtgatcgtcgtgattatctgcatcaagctgaagcacaccaagaagaggcagatctacaccgacatcgagatgaacaggctgggcaagtgatgattcgaa

SEQ ID NO: 283 - DNA, 120 bp, Artificial Sequence
  GeneArt_Seq101
ggcgccgtgattaccgtggtcgtgatcgtcgtgattatctgcatcaagctgaagcacaccaagaagaggcagatcgccgccgccgccgccgccaacaggctgggcaagtgatgattcgaa

SEQ ID NO: 284 - DNA, 1064 bp, Artificial Sequence
  GSC11206
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcagagctgcaaccggtggagagctgtgcggaggcgcaggacggggagctggacggggccctgtggctgggcgtgctgagactgctgctgttcaagctgctcctgttcgacctgctcctcaagtggatcttctcctcggtggtggacctgaagcagaccatcatccccgactacaggaacatgatcggacagggggcctagtgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 285 - protein, 70 aa, Artificial Sequence
  GSC11206
LSLSPELQPVESCAEAQDGELDGALWLGVLRLLLFKLLLFDLLLKWIFSSVVDLKQTIIPDYRNMIGQGA

SEQ ID NO: 286 - protein, 470 aa, Artificial Sequence
  GSC314
MESPAQLLFLLLLWLPDTQAEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 287 - DNA, 375 bp, Artificial Sequence
  GSC3836
ctgtctccgggtaaatgatgaaagcttgcggccgcgcttagcgatatcgaattctctagaggtacccccggggggcccctcgagttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 288 - protein, 5 aa, Artificial Sequence
  GSC3836 / GSC3848
LSPGK

SEQ ID NO: 289 - DNA, 884 bp, Artificial Sequence
  GSC3848
ctgtctccgggtaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcagagctgcaaccggtggagatattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 290 - protein, 76 aa, Artificial Sequence
  GSC3899 / GSC4398 / GSC4401 / GSC4402
LSLSPELQPVESCAEAQDGELDGLWTTITIFITLFLLSVCYSATVTFFKVKWIFSSVVDLKQTIIPDYRNMIGQGA

SEQ ID NO: 291 - protein, 42 aa, Artificial Sequence
  GSC5854 / GSC5855 / GSC5857 / GSC5856
LSLSPAELQPVEALWLGVLRLLLFKLLLFDLLLASAHHHHHH

SEQ ID NO: 292 - protein, 74 aa, Artificial Sequence
  GSC6030 / GSC6048 / GSC6047 / GSC6046
LSLSPELQPVESCAEAQDGELDALWLGVLRLLLFKLLLFDLLLTFFKVKWIFSSVVDLKQTIIPDYRNMIGQGA

SEQ ID NO: 293 - protein, 78 aa, Artificial Sequence
  GSC7057 / GSC7058 / GSC7056 / GSC7059 / GSC9763 / GSC9764 / GSC9765 / GSC9768 / GSC9770 / GSC9949 / GSC9950
LSPGGGGSPPEDPPDSKNTLVLFGAGFGAVITVVVIVVIIKCFCKHRSCFRRNEASRETNNSLTFGPEEALAEQTVFL

SEQ ID NO: 294 - DNA, 708 bp, Artificial Sequence
  GSC315
atggagagccctgcccagctgctgttcctgctgctgctgtggctgcctgacggagtccatgccgacatccagatgacccagtccccctccagcctgtctgcctccgtgggcgaccgggtgaccatcacctgccgggcctcccaggacgtgaacaccgccgtggcctggtatcagcagaagcctggcaaggcccctaagctgctgatctactccgcctccttcctgtactccggcgtgccttcccggttctccggctcccggtccggcaccgacttcaccctgaccatctcctccctgcagcctgaggacttcgccacctactactgccagcagcactacaccacccctcctaccttcggccagggcaccaaggtggagatcaagcggaccgtggccgctccttccgtgttcatcttccctccctccgacgagcagctgaagagcggcaccgccagcgtggtgtgcctgctgaacaacttttaccctcgggaggccaaggtgcagtggaaggtggacaacgccctgcagtccggcaactcccaggaatccgtcaccgagcaggactccaaggacagcacctactccctgtcctccaccctgaccctgtccaaggccgactacgagaagcacaaggtgtacgcctgcgaggtgacccaccagggcctgtccagccctgtgaccaagtccttcaaccggggcgagtgctga

SEQ ID NO: 295 - protein, 235 aa, Artificial Sequence
  GSC315
MESPAQLLFLLLLWLPDGVHADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 296 - DNA, 1428 bp, Artificial Sequence
  GSC5607
atgcggagccctgcccagctgctgttcctgctgctgctgtggatccctggcaccaacgcccaggtgcagctgcagcagtctggcgccgaactggccagaccaggcgccagcgtgaagatgagctgcaaggccagcggctacaccttcacccggtacaccatgcactgggtgaaacagcggcctggacagggcctggaatggatcggctacatcaaccccagccggggctacaccaactacaaccagaagttcaaggacaaggccaccctgaccaccgacaagagcagcagcaccgcctacatgcagctgagcagcctgaccagcgaggacagcgccgtgtactactgcgcccggtactacgacgaccactactgcctggactactggggccagggcaccaccctgaccgtgagcagcgcgtcgaccaagggccccagcgtgttcccgctagcccccagcagcaagagcaccagcggcggcacagccgccctgggctgcctggtgaaggactacttccccgagcccgttaccgtgtcctggaactctggagccctgacctccggcgtgcacaccttccccgccgtgctccagagcagcggcctgtacagcctgagcagcgtggtgacagtgcccagcagcagcctgggaacccagacctacatctgcaacgtgaaccacaagcccagcaacaccaaggtggacaagaaggtggagcccaagagctgcgacaaaactcacacctgcccaccctgccctgcccctgagctgctgggcggaccctccgtgttcctgttcccccccaagcccaaggacaccctgatgatcagccggacccccgaggtgacctgcgtggtggtggacgtgagccacgaggaccctgaggtgaagttcaattggtacgtggacggcgtggaggtgcacaacgccaagaccaagccccgggaggaacagtacaacagcacctaccgggtggtgtccgtgctgaccgtgctgcaccaggactggctgaacggcaaggaatacaagtgcaaggtctccaacaaggccctgcctgcccccatcgaaaagaccatcagcaaggccaagggccagccgcgcgaaccgcaggtgtacaccctgccccccagccgggaagagatgaccaagaaccaggtgaaactggtgtgcctggtgacaggcttctaccccagcgatatcgccgtggaatgggagagcagcggccagcctgagaacaactactacaccaccccccccatgctggacagcgacggcagcttcagcctggtgtcctggctgaacgtggacaagagccggtggcagcagggcaacatcttcagctgcagcgtgatgcacgaggccctgcacaaccggttcacccagaagtccctgagcctgagcccgggtaaacatcaccatcaccatcactga

SEQ ID NO: 297 - protein, 475 aa, Artificial Sequence
  GSC5607
MRSPAQLLFLLLLWIPGTNAQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPGKHHHHHH

SEQ ID NO: 298 - protein, 469 aa, Artificial Sequence
  GSC5644
MRSPAQLLFLLLLWIPGTNAQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVKLVCLVTGFYPSDIAVEWESSGQPENNYYTTPPMLDSDGSFSLVSWLNVDKSRWQQGNIFSCSVMHEALHNRFTQKSLSLSPGK

SEQ ID NO: 299 - protein, 76 aa, Artificial Sequence; identical to SEQ ID NO: 290
  GSC5537

SEQ ID NO: 300 - protein, 495 aa, Artificial Sequence
  GSC5608
MRSPAQLLFLLLLWIPGTNAEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRGGGGTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPEVATFPPSRDELTKNQVTLVCLVTGFYPSDIAVEWESNGQPENNYKTDPPLLESDGSFALSSRLRVDKSRWQQGNVFSCSVMHEALHNHYTQKSLSLSPGK

SEQ ID NO: 301 - protein, 76 aa, Artificial Sequence; identical to SEQ ID NO: 290
  GSC5538

SEQ ID NO: 302 - DNA, 702 bp, Artificial Sequence
  GSC5540
atgcggagccctgcccagctgctgttcctgctgctgctgtggatccctggcaccaacgcccagattgtgctgacccagagccccgccatcatgtctgccagccctggcgagaaagtgaccatgacctgcagcgccagcagcagcgtgtcctacatgaactggtatcagcagaagtccggcaccagccccaagcggtggatctacgacaccagcaagctggcctccggcgtgcccgcccactttagaggcagcggcagcggcacctcctactccctgaccatcagcggcatggaagccgaggacgccgccacctactactgccagcagtggtccagcaaccccttcaccttcggctccggcaccaaactggaaattaaccgtacggtggccgctcccagcgtgttcatcttcccccccagcgacgagcagctgaagagcggcaccgcctccgtggtgtgcctgctgaacaacttctacccccgggaggccaaggtgcagtggaaggtggacaacgccctccagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagtgctga

SEQ ID NO: 303 - protein, 233 aa, Artificial Sequence
  GSC5540
MRSPAQLLFLLLLWIPGTNAQIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 304 - DNA, 1522 bp, Artificial Sequence
  GSC281
gaattgtacctgcccgtacataaggtcaataggggtgaatcaacggaaaagtcccattggagccaagtacactgcgtcaatagggactttccattgggttttgcccagtacataaggtcaataggggatgagtcaatgggaaaaacccattggagccaagtacactgactcaatagggactttccattgggttttgcccagtacataaggtcaatagggggtgagtcaacaggaaagttccattggagccaagtacattgagtcaatagggactttccaatgggttttgcccagtacataaggtcaatgggaggtaagccaatgggtttttcccattactggcacgtatactgagtcattagggactttccaatgggttttgcccagtacataaggtcaataggggtgaatcaacaggaaagtcccattggagccaagtacactgagtcaatagggactttccattgggttttgcccagtacaaaaggtcaatagggggtgagtcaatgggtttttcccattattggcacgtacataaggtcaataggggtgagtcattgggtttttccagccaatttaattaaaacgccatgtactttcccaccattgacgtcaatgggctattgaaactaatgcaacgtgacctttaaacggtactttcccatagctgattaatgggaaagtaccgttctcgagccaatacacgtcaatgggaagtgaaagggcagccaaaacgtaacaccgccccggttttccctggaaattccatattggcacgcattctattggctgagctgcgttcacgtgggtataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcgacggatcccgtactatgaggtgtggcaggcttgagatctggccatacacttgagtgacaatgacatccactttgcctttctctccacaggtgtccactcccacgtccaactgcagctcggttcgatcgataattaattaagctagcggcgccgttaacaccggttgatcaaagcttgcggccgcgcttagcgatatcgaattctctagaggtacccccggggggcccctcgagttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatctta

SEQ ID NO: 305 - DNA, 132 bp, Artificial Sequence
  GSC3469
aacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 306 - DNA, 470 bp, Artificial Sequence
  GSC23
cgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggggcaggataatatatggtagggttcatagccagagtaacctttttttttaatttttattttattttatttttgagatcag

SEQ ID NO: 307 - DNA, 528 bp, Artificial Sequence
  GSC4340
ggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgagacctgaggggacag

SEQ ID NO: 308 - DNA, 528 bp, Artificial Sequence
  GSC4222
ggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccctgtccttattgcacag

SEQ ID NO: 309 - DNA, 528 bp, Artificial Sequence
  GSC2617
ggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccctgggaggattgcacag

SEQ ID NO: 310 - DNA, 528 bp, Artificial Sequence
  GSC4335
ggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaaccgtctccttctgggacag

SEQ ID NO: 311 - DNA, 546 bp, Artificial Sequence
  GSC2332
ggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctgctacctggctcag

SEQ ID NO: 312 - DNA, 36 bp, Artificial Sequence
  Primer GlnPr2160
ccaagctttcatcatttgcccggagacagggagagg

SEQ ID NO: 313 - DNA, 42 bp, Artificial Sequence
  Primer GlnPr2161
ccaagctttcatcatttaccgggagacagggagaggctcttc

SEQ ID NO: 314 - DNA, 49 bp, Artificial Sequence
  Primer GlnPr2162
gtcctcggggggagatccaccaccacctgtcccagaaggagacggttag

SEQ ID NO: 315 - DNA, 49 bp, Artificial Sequence
  Primer GlnPr2163
gtcctcggggggagatccaccaccacctgtgcaatcctcccagggttag

SEQ ID NO: 316 - DNA, 49 bp, Artificial Sequence
  Primer GlnPr2164
gtcctcggggggagatccaccaccacctgtgcaataaggacagggttag

SEQ ID NO: 317 - DNA, 49 bp, Artificial Sequence
  Primer GlnPr2165
gtcctcggggggagatccaccaccacctgtcccctcaggtctcggttag

SEQ ID NO: 318 - DNA, 49 bp, Artificial Sequence
  Primer GlnPr2166
gtcctcggggggagatccaccaccacctgagccaggtagcagggaaggg

SEQ ID NO: 319 - DNA, 705 bp, Artificial Sequence
  Construct GSC3704
atggaaagcccagcccagctgctgttcctgctgctgctgtggctgcccgacggggtgcacgctgagatcgtgctgacacagagccccgccaccctgtctctgagccctggcgaaagagccaccctgagctgtagagccagcagcagcgtgtcctacatgcactggtatcagcagaagcccggccaggcgccgcgcccgtggatttatgcgaccagcaatcgggccacaggcatccctgccagattttctggcagcggctccggcaccgactacaccctgaccatctccagcctggaacccgaggacttcgccgtgtactactgccagcagtggtccagcaacccctggacatttggccagggcaccaaagtggaaattaaacgtacggtggccgctcccagcgtgttcatcttcccccccagcgacgagcagctgaagagcggcaccgcctccgtggtgtgcctgctgaacaacttctacccccgggaggccaaggtgcagtggaaggtggacaacgccctccagagcggcaacagccaggaaagcgtcaccgagcaggacagcaaggactccacctacagcctgagcagcaccctgaccctgagcaaggccgactacgagaagcacaaggtgtacgcctgcgaggtgacccaccagggcctgtccagccccgtgaccaagagcttcaaccggggcgagtgctga

SEQ ID NO: 320 - protein, 234 aa, Artificial Sequence
  Construct GSC3704
MESPAQLLFLLLLWLPDGVHAEIVLTQSPATLSLSPGERATLSCRASSSVSYMHWYQQKPGQAPRPWIYATSNRATGIPARFSGSGSGTDYTLTISSLEPEDFAVYYCQQWSSNPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 321 - DNA, 1094 bp, Artificial Sequence; identical to SEQ ID NO: 55
  Construct GSC10488

SEQ ID NO: 322 - protein, 80 aa, Artificial Sequence; identical to SEQ ID NO: 127
  Construct GSC10488

SEQ ID NO: 323 - DNA, 1094 bp, Artificial Sequence; identical to SEQ ID NO: 55
  Construct GSC10487

SEQ ID NO: 324 - protein, 80 aa, Artificial Sequence; identical to SEQ ID NO: 127
  Construct GSC10487

SEQ ID NO: 325 - DNA, 53 bp, Artificial Sequence
  GlnPr1487
ctgctgctgtggctgcccgacggggtgcacgctgagatcgtgctgacacagag

SEQ ID NO: 326 - DNA, 35 bp, Artificial Sequence; identical to SEQ ID NO: 6
  GlnPr1491

SEQ ID NO: 327 - DNA, 31 bp, Artificial Sequence
  GlnPr1452
acgtatcgattcatcacttgccgggggacag

SEQ ID NO: 328 - DNA, 53 bp, Artificial Sequence
  GlnPr1488
ctgctgctgtggctgcccgacggggtgcacgctcaggtcacactgaaagagtc

SEQ ID NO: 329 - DNA, 1470 bp, Artificial Sequence
  pGLEX41_HC-GGC-I4(SV40ter)-B7-B7(18)-6His
ctgtctccgggcaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggggcaggataatatatggtagggttcatagccagagtaacctttttttttaatttttattttattttatttttgagatcagttcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccaccgtgatcgtcgtgattatcgccagcgcccatcaccaccaccatcattgatgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 330 - protein, 46 aa, Artificial Sequence
  pGLEX41_HC-GGC-I4(SV40ter)-B7-B7(18)-6His
LSLSRGGGSPPEDPPDSKNTLVLFGAGFGATVIVVIIASAHHHHHH

SEQ ID NO: 331 - DNA, 1135 bp, Artificial Sequence
  pGLEX41_HC-GGC-I4(polyA)-B7-B7(18)-6His
ctgtctccgggcaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatacttgtttattgcagcttataatggttacaaataaagcaatagcatcacaaatttcacaaataaagcatttttttcactgcattctagttgtggtttgtccaaactcatcaatgtatcttatcatgtctgtatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccaccgtgatcgtcgtgattatcgccagcgcccatcaccaccaccatcattgatgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 332 - protein, 45 aa, Artificial Sequence
  pGLEX41_HC-GGC-I4(polyA)-B7-B7(18)-6His
LSPGGGGSPPEDPPDSKNTLVLFGAGFGATVIVVIIASAHHHHHH

SEQ ID NO: 333 - DNA, 1123 bp, Artificial Sequence
  pGLEX41_HC-GGC-I4(polyA)-B7-B7(18)-KCFCK
ctgtctccgggcaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatacttgtttattgcagcttataatggttacaaataaagcaatagcatcacaaatttcacaaataaagcatttttttcactgcattctagttgtggtttgtccaaactcatcaatgtatcttatcatgtctgtatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccaccgtgatcgtcgtgattatcaagtgcttctgcaagtgatgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 334 - protein, 41 aa, Artificial Sequence
  pGLEX41_HC-GGC-I4(polyA)-B7-B7(18)-KCFCK
LSPGGGGSPPEDPPDSKNTLVLFGAGFGATVIVVIIKCFCK

SEQ ID NO: 335 - DNA, 1141 bp, Artificial Sequence
  pGLEX41_HC-GGC-I4(polyA)-B7-B7(20)-6His
ctgtctccgggcaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatacttgtttattgcagcttataatggttacaaataaagcaatagcatcacaaatttcacaaataaagcatttttttcactgcattctagttgtggtttgtccaaactcatcaatgtatcttatcatgtctgtatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccaccgtggtcgtgatcgtcgtgattatcgccagcgcccatcaccaccaccatcattgatgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 336 - protein, 47 aa, Artificial Sequence
  pGLEX41_HC-GGC-I4(polyA)-B7-B7(20)-6His
LSPGGGGSPPEDPPDSKNTLVLFGAGFGATVVVIVVIIASAHHHHHH

SEQ ID NO: 337 - DNA, 1129 bp, Artificial Sequence
  pGLEX41_HC-GGC-I4(polyA)-B7-B7(20)-KCFCK
ctgtctccgggcaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgatacttgtttattgcagcttataatggttacaaataaagcaatagcatcacaaatttcacaaataaagcatttttttcactgcattctagttgtggtttgtccaaactcatcaatgtatcttatcatgtctgtatcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccaccgtggtcgtgatcgtcgtgattatcaagtgcttctgcaagtgatgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 338 - protein, 43 aa, Artificial Sequence
  pGLEX41_HC-GGC-I4(polyA)-B7-B7(20)-KCFCK
LSPGGGGSPPEDPPDSKNTLVLFGAGFGATVVVIVVIIKCFCK

SEQ ID NO: 339 - DNA, 1458 bp, Artificial Sequence
  pGLEX41_HC-GGC-I4(SV40ter)-B7-B7(18)-KCFCK
ctgtctccgggcaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggggcaggataatatatggtagggttcatagccagagtaacctttttttttaatttttattttattttatttttgagatcagttcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccaccgtgatcgtcgtgattatcaagtgcttctgcaagtgatgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 340 - protein, 41 aa, Artificial Sequence; identical to SEQ ID NO: 334
  pGLEX41_HC-GGC-I4(SV40ter)-B7-B7(18)-KCFCK

SEQ ID NO: 341 - DNA, 1476 bp, Artificial Sequence
  pGLEX41_HC-GGC-I4(SV40ter)-B7-B7(20)-6His
ctgtctccgggcaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggggcaggataatatatggtagggttcatagccagagtaacctttttttttaatttttattttattttatttttgagatcagttcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccaccgtggtcgtgatcgtcgtgattatcgccagcgcccatcaccaccaccatcattgatgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 342 - protein, 47 aa, Artificial Sequence; identical to SEQ ID NO: 336
  pGLEX41_HC-GGC-I4(SV40ter)-B7-B7(20)-6His

SEQ ID NO: 343 - DNA, 1464 bp, Artificial Sequence
  pGLEX41_HC-GGC-I4(SV40ter)-B7-B7(20)-KCFCK
ctgtctccgggcaaatgatgaaagcttggtaggtgatcctcctgctgctttggttcagggttttgcttgaggggggggggtggtgatttccttgccatgggcagactgagcagaaaaggccattgggaccatgttctgaatgcctccacctcaaccaccggccggtaggaccaaagccaccccgtgttttctcaggatctcttttcccagggagatccctcggcccaaagagggagatggcaatgctggatgtgtgcacaataattcaacaggcattggaacttcagcatcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggggcaggataatatatggtagggttcatagccagagtaacctttttttttaatttttattttattttatttttgagatcagttcgatgctgaatgcaattaacaatgctcaagcagaacccccggctccatcagcacagtgcaggaccaaaccccatgctgcagcagtggggctgtctgtacggggtgggcaatgggaaccggggtctgctggggctcctgctgcttcagtgctgccatgcagccacacatcctgagagctgaaagggtcggcgtcctcacctggtgcacaccgtagctctgccccacagctttaaggcacctggctaacctctgcgcttcttcccttccctcctccctggctcaggtggtggtggatctccccccgaggacccccctgactccaagaataccctggtgctgttcggcgctggcttcggcgccaccgtggtcgtgatcgtcgtgattatcaagtgcttctgcaagtgatgatgattcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgt

SEQ ID NO: 344 - protein, 43 aa, Artificial Sequence; identical to SEQ ID NO: 338
  pGLEX41_HC-GGC-I4(SV40ter)-B7-B7(20)-KCFCK

SEQ ID NO: 345 - DNA, 9 bp, Artificial Sequence
  Splice donor consensus
maggtragt

SEQ ID NO: 346 - DNA, 12 bp, Artificial Sequence
  Splice acceptor consensus
  misc_feature (7)..(7): poly (Y) tract comprising at least 15 nucleotides and an abundance of pyrimidine (C or T) bases
  misc_feature (8)..(8): n is a, c, g, or t
ctrayynncagg

SEQ ID NO: 347 - DNA, 6 bp, Artificial Sequence
  Branch Point Consensus
ctrayy

(19) United States
(12) Patent Application Publication
(10) Pub. No.: US 2017/0350886 A1
(43) Pub. Date: Dec. 7, 2017

(54) EXPRESSION CONSTRUCTS AND METHODS FOR SELECTING HOST CELLS EXPRESSING POLYPEPTIDES

(71) Applicant: GLENMARK PHARMACEUTICALS S.A., La Chaux-de-Fonds (CH)

(72) Inventors: Christel AEBISCHER-GUMY, La Chaux-de-Fonds (CH); Pierre MORETTI, La Chaux-de-Fonds (CH); Martin BERTSCHINGER, La Chaux-de-Fonds (CH)

(73) Assignee: GLENMARK PHARMACEUTICALS S.A., La Chaux-de-Fonds (CH)

(21) Appl. No.: 15/121,819

(22) PCT Filed: Mar. 2, 2015
(86) PCT No.: PCT/EP2015/054331

§ 371 Date: Aug. 26, 2016

(30) Foreign Application Priority Data
Feb. 28, 2014 (EP) 14157324.6

(51) Int. Cl. G01N 33/569; C07K 16/00; C12N 15/85; G01N 33/68; C12P 21/00

(52) U.S. Cl. CPC G01N 33/56966; G01N 33/6854; C12P 21/00; C12N 15/85; C07K 16/00; C07K 2317/31; C07K 2317/622; C07K 2317/52; C12N 2830/42; C12N 2830/50

(57) ABSTRACT

The invention relates to an expression construct for the soluble expression of polypeptides in host cells and for the expression of polypeptides on the surface of host cells, using alternative splicing. The amount of cell membrane expression of polypeptides such as antibodies, antibody fragments and bispecific antibodies can be correlated directly to the amount of soluble polypeptide expressed. In the case of bispecific antibodies, cell membrane expression of heterodimer and homodimer products can be correlated directly to the soluble expression of these products, thereby aiding selection of a desired producer clone.

### FIELD OF THE INVENTION

The present invention relates to expression constructs and methods for expressing a fraction of a secreted protein of interest on the surface of eukaryotic cells using alternate splicing. The present invention also relates to methods of selecting cell(s) which express a secreted protein of interest at a desired level by detecting the membrane expression level of the protein of interest. The present invention further relates to the selection of cell(s) expressing secreted heteromultimeric proteins of interest by detecting the membrane expression level of the heteromultimeric protein of interest.

### BACKGROUND OF THE INVENTION

In order to produce a protein in a eukaryotic cell, the DNA coding for this protein has to be transcribed into a messenger RNA (mRNA) which will in turn be translated into a protein. The mRNA is first transcribed in the nucleus as pre-mRNA, containing introns and exons. During the maturation of the pre-mRNA into mature mRNA, the introns are cut out (“spliced”) by cellular machinery called the spliceosome. The exons are fused together and the mRNA is modified by the addition of a so called CAP at its 5′end and a poly adenylation (poly(A)) tail at its 3′ end. The mature mRNA is exported to the cytoplasm and serves as template for the translation of proteins.

Alternate splicing is a term describing that the same transcript might be spliced in different fashions leading to different mature mRNAs and in some cases to different proteins. This mechanism is used in nature to change the expression level of proteins or in order to modify the activity of certain proteins during development (Cooper T A & Ordahl C P (1985), J Biol Chem, 260(20): 11140-8). Alternate splicing is usually controlled by complex interactions of many factors (Orengo J P et al., (2006) Nucleic Acids Res, 34(22): e148).

Alternate splicing enables the production of two (or more) different RNA transcripts from the same DNA template. This can be used to produce two (or more) isoforms of the same protein or polypeptide. Throughout the specification the terms protein and polypeptide will be used interchangeably.

In nature, this is a highly controlled process, used for example during the process of antibody production by activated B cells in the human immune system. Most of the antibody is secreted into the extracellular space, but a fraction of the produced antibody is redirected from the secretory pathway to the outer cellular membrane in the form of membrane bound isoforms.

The membrane bound isoforms have the same amino acid sequence and structure as the antibody secreted into the extracellular space. The difference is a C-terminal extension of the secreted antibody's heavy chain with a transmembrane region comprising a transmembrane domain. In B cells this domain can have a length of between approximately 40 to 75 amino acids (Major J G et al., (1996) Mol. Immunol. 33: 179-87).

Expression systems for the production of recombinant polypeptides are well-known in the art. For the production of polypeptides and proteins used in pharmaceutical preparations mammalian host cells such as CHO, BHK, NS0, Sp2/0, COS, HEK and PER.C6 are generally used. For large scale production of therapeutic proteins a high producer cell line has to be generated. After transfection of the host cell line with a gene encoding the polypeptide of interest, a number of clones with different characteristics are obtained and selected for. This is routinely carried out by using, for example, a selectable marker, gene amplification and/or reporter molecules. The selection of an appropriate clone with desired properties e.g. a high producer clone, is a time consuming, often non-routine and therefore expensive process.

For the expression of heteromultimeric proteins, such as bispecific antibodies, the generation of a suitable host cell line becomes even more complicated. The protein subunits that make up the multimer can be expressed in separate cell lines and then brought together for association into the multimeric protein or alternatively the different subunits can be expressed in the same cell line. Expression of the subunits in the same cell line is associated with disadvantages wherein not all protein subunits will associate into the correct form, resulting in a mixture of different species. For the generation of bispecifc antibodies it is common for significant levels of homodimers to be produced rather than the desired heterodimer and this greatly impacts bispecific antibody production yields. Whilst the unwanted homodimers can be removed by various purification techniques, it is desirable to have a host cell line in which heterodimers are expressed at higher levels than homodimers to reduce the time and costs wasted on downstream processing.

Hence there is a need for an expression system that can be used to select for cell clone(s) that express a product of interest at a high level i.e. quantitative selection. There is also a need to be able to select for cell clone(s) that express a product of interest of a desired quality i.e. qualitative selection, for instance the expression of a heteromultimeric protein of interest.

### SUMMARY OF THE INVENTION

The present invention relates to an expression construct or set of constructs for the expression by alternate splicing of a soluble polypeptide of interest, wherein a portion of the soluble peptide is secreted into the extra cellular space and a portion is the displayed on the outer membrane of a cell(s).

The present invention also comprises methods for the selection of host cells comprising one or more constructs according to the present invention, which display on their outer membrane a polypeptide of interest at a desired level.

The present invention further relates to the selection of cell(s) expressing secreted heterodimeric proteins of interest by detecting the membrane display of the heterodimeric protein of interest on a cell(s) comprising one or more constructs according to the present invention.

In a first aspect, the present invention provides an expression construct comprising in a 5′ to 3′ direction:

a promoter;
a first exon encoding a polypeptide of interest;
a splice donor site, an intron and a splice acceptor site, wherein a first stop codon is located between the splice donor site and the splice acceptor site within said intron;
a second exon encoding a transmembrane region which is a modified immunoglobulin transmembrane region or a non-immunoglobulin transmembrane region;
a second stop codon; and
a polyadenylation site;
wherein upon entry into a host cell, transcription of the first and second exons results in expression of the polypeptide of interest and display of a proportion of the polypeptide of interest on the outer membrane of the host cell.

In accordance with another aspect of the present invention the transmembrane region encoded by the second exon of the construct comprises at least 4, 5, 6, 7, 8, 9, 10, 11, 12, 13, 14, 15, 16, 17, 18, 19, 20, 21 or 22 amino acid residues.

In accordance with another aspect of the present invention the transmembrane region encoded by the second exon of the construct comprises no more than 48, 47, 46, 45, 44, 43, 42, 41, 40, 39, 38, 37, 36, 35, 34, 33, 32, 31, 30, 29, 28, 27, 26 amino acid residues.

In particular the transmembrane region may comprise between 17 and 29 residues, 19 and 26 residues or 21 and 24 residues.

In accordance with the present invention, the modified immunoglobulin transmembrane region or modified non-immunoglobulin transmembrane region may be any modified version of a naturally occurring immunoglobulin or non-immunoglobulin transmembrane region.

The inventors have modified a diverse group of transmembrane regions, so as to alter their properties and in particular modulate their integration into the cell membrane and hence the display of the polypeptide of interest. In particular the amino acid composition of the transmembrane region may be altered so as to reduce or increase the number of non-hydrophobic residues therein by one or more residues, as well as increase or decrease the size of the transmembrane region.

The inventors of the present invention have surprisingly found that use of a non-immunoglobulin transmembrane region for instance a murine B7-1 transmembrane region (SEQ ID NO: 173) leads to a high level of cell membrane expression compared to the levels observed when using the IgG1 transmembrane region.

The inventors have also tested other transmembrane regions from ACLV1 (NP_001070869.1) SEQ ID NO: 174, ANTR2 (NP_477520.2) SEQ ID NO: 175, CD4 (NP_000607.1) SEQ ID NO: 176, PTPRM (NP_002836.3) SEQ ID NO: 177, TNR5 (NP_001241.1) SEQ ID NO: 178, ITB1 (NP_596867.1) SEQ ID NO: 179, IGF1R (NP_000866.1) SEQ ID NO: 181, 1B07 (NP_005505.2) SEQ ID NO: 180, TRMB (NP_000352.1) SEQ ID NO: 182, IL4RA (NP_000409.1) SEQ ID NO: 183, LRP6 (NP_002327.2) SEQ ID NO: 184, GpA (NP_002090) SEQ ID NO: 185, PTCRA (NP_001230097.1) SEQ ID NO: 186 as well as modified versions of these transmembrane regions, and shown these to be suitable for use in the construct(s) according to the present invention.

In accordance with another aspect of the present invention the first stop codon is located 3′ of the splice donor site of the intron.

In one embodiment the present invention also provides methods for altering the splice ratio observed for a given construct, so that a sufficient amount of polypeptide is displayed on the cell membrane to enable cell selection, whilst most of the polypeptide is expressed solubly.

The inventors have tested the different components of the claimed expression constructs allowing them to modulate the membrane display of the protein of interest.

According to the present invention, the level of soluble polypeptide expression can be increased by including a poly(A) site in the intron of the expression construct as described herein.

According to an aspect of the present invention the construct may comprise a constitutive intron positioned between the promoter and the first exon.

In accordance with the present invention the strength of the splice acceptor and the splice donor may be modified so as to increase or decrease the amount of the protein of interest displayed on the cell membrane.

In particular the consensus sequence of the splice donor site may be modified, by decreasing or increasing the % identity or similarity of the sequence of the splice donor site present in the construct to the consensus splice donor site sequence (C/A)AGGT(A/G)AGT (SEQ ID NO: 345).

In a further embodiment, the consensus sequence of the splice donor site is modified in order to decrease the splice donor strength. The consensus sequence of the splice donor site can be modified by alternative codon usage, e.g. by replacing AAA coding for lysine with an AAG coding for lysine. This will reduce the level of membrane expression. Conversely by increasing the % identity or similarity of the splice donor site to the consensus splice donor site sequence (C/A)AGGT(A/G)AGT (SEQ ID NO: 345), this will increase the strength of the splice donor site in the construct and increase the level of membrane expression.

According to an aspect of the present invention, the splice donor site overlaps the 3′ end of the first exon and the 5′ end of the intron and the splice acceptor site of the intron is located at the 3′ end of the intron.

According to an aspect of the present invention the first stop codon is located 3′ of the splice donor site.

According to the intron of the expression construct optionally comprises a poly(Y) tract included in the splice acceptor site. The Y content of the poly(Y) tract included in the splice acceptor site of the intron of the expression construct can be modified. Altering the number of pyrimidine bases (Ys) in the poly(Y) tract can be used to decrease or increase cell membrane expression of the polypeptide of interest. For a polypeptide of interest with high level of membrane expression by the cell, reducing the number of Ys in the poly(Y) tract will reduce the level of membrane expression. For a polypeptide with low level of membrane expression by the cell, increasing the number of Ys in the poly(Y) tract will increase the level of membrane expression.

In particular the poly(Y) content of the splice acceptor may be decreased so as to decrease the strength of the splice acceptor site, by altering the poly(Y) content of the splice acceptor site, reducing the membrane display of the polypeptide of interest.

Alternatively the poly(Y) content of the splice acceptor may be increased so as to increase the strength of the splice acceptor site, by altering the poly(Y) content of the splice acceptor site, so increasing the membrane display of the polypeptide of interest.

In addition to the modifications outlined above, the alternate splicing event leading to membrane displayed polypeptide can also be influenced by modifying the DNA (and hence the RNA) sequence of the branch point region. A nucleotide of the branch point region initiates the splice event by attacking the first nucleotide of the intron at the 5′ splice site, thus forming a lariat intermediate. Changing the sequence of the branch point region relative to the consensus sequence (CTRAYY SEQ ID NO: 347) can have an impact on the efficacy of the initiation of the splice event and thus on the ratio of secreted polypeptide versus membrane displayed polypeptide.

Further, the length of the intron can impact the splice ratio. It has been demonstrated that the likelihood of including an alternate exon in CD44 increases when the intron directly upstream of the exon was shortened (Bell, M., Cowper, A., Lefranc, M., Bell, J. and Screaton, G. (1998). Influence of Intron Length on Alternative Splicing of CD44 Mol Cell Biol. 18(10): 5930-5941.). Hence a further means to modify the constructs is to shorten the length of the intron in the alternate splicing constructs so as to to increase the fraction of membrane displayed polypeptide or vice versa.

An additional mechanism to influence the splice event is the co-expression of RNA-binding proteins leading to exon inclusion or skipping. For example the proteins CUG-BP (Uniprot Acc.-No.: Q5F3T7) and muscle-blind like 3 (MBNL) (Uniprot Acc.-No.: Q5ZKW9) have been shown to influence the splice ratio in a construct expressing EGFP and dsRED (Orengo et al., 2006).

The present invention therefore also provides a further modified construct comprising expressible ORFs encoding RNA-binding proteins which cause exon inclusion or skipping. As well as methods involving the co-transfection of constructs according to the present invention, comprising expressible ORFs encoding RNA-binding proteins which cause exon inclusion or skipping and/or separate constructs comprising such ORFs.

In addition most membrane proteins pass through the endoplasmic reticulum (ER) and Golgi apparatus before reaching the cell surface. Export from the ER is a selective process that is mediated by coatomer complex II (COPII) transport vesicles that bud from sites of ER exit. Interactions between components of the COPII transport vesicles and short amino acid sequences with linear di-acid, hydrophobic and aromatic motifs or structural motifs in the cytoplasmic domain of membrane-anchored proteins concentrate cargo proteins at ER exit sites and enhance cargo recruitment into COPII vesicles. These short linear or structural amino acid sequence motifs are called ER exportation signal.

Another approach to adjust the membrane display of a protein of interest therefore is to include an ER exportation signal or not, so as to modify ER passage of the polypeptide of interest fused with the transmembrane region. Modification of the ER exportation signal comprised within the construct so as to increase ER exportation is useful for increasing the membrane display of proteins which have low half-life or other stability or degradation issues and vice versa.

The inventors of the present invention have found that the amount of polypeptide of interest displayed on the cell membrane is directly proportional to the level of expression of soluble polypeptide. Therefore host cells expressing the polypeptide of interest at high titre display more polypeptide on the membrane than host cells expressing the polypeptide at low titre. This enables the straightforward identification and isolation of high producing recombinant host cells.

In an aspect of the present invention, the polypeptide encoded by the expression construct can be part of a protein multimer for instance a heteromultimeric polypeptide such as recombinant antibody or fragments thereof. The antibody fragments may be selected from the list consisting of: Fab, Fd, Fv, dAb, F(ab′)₂ and scFv. In a preferred embodiment, the polypeptide expressed by the expression construct can be an antibody heavy chain or fragments thereof.

In a further aspect of the present invention, the expression construct can be used for the expression of a bispecific antibody in a host cell. In one embodiment, the polypeptide expressed is an antibody heavy chain. Alternatively, the polypeptide expressed is a fragment of antibody linked to an antibody Fc region. The antibody fragment may be selected from the list consisting of: Fab, Fd, Fv, dAb, F(ab′)₂ and scFv. Preferably the antibody fragment is a Fab or a scFv. More preferably the antibody fragment is a scFv. To effect expression of a bispecific antibody, a separate expression construct may also be provided for the expression of an antibody light chain. Co-expression of the expression constructs coding for an antibody heavy chain and an antibody fragment-Fc with an expression construct coding for an antibody light chain in host cells, results in the expression of bispecific antibody. As discussed above, expression of a bispecific antibody in host cells results in a number of unwanted homodimeric species besides the desired heterodimer. In a preferred embodiment of the invention, cell membrane display of these bispecific antibody components enables the straightforward selection of a host cell expressing predominantly heterodimeric antibodies.

The present invention also provides a method for altering the splice ratio so that a sufficient amount of polypeptide is displayed on the cell membrane to enable cell selection, whilst most of the polypeptide is expressed solubly.

In accordance with this aspect of the present invention the method involves measuring the membrane display of the polypeptide of interest and then modifying the components of the construct based upon the observed membrane display of the polypeptide of interest so as to increase or decrease membrane display so as to allow the better selection of cell(s) expressing the polypeptide of interest.

The present invention also provides a method to select a cell(s) comprising at least one construct according to the present invention, involving the selection of cell(s) by detecting the membrane expression level of the protein of interest or a heteromultimeric protein comprising the protein of interest.

### BRIEF DESCRIPTION OF THE FIGURES

FIG. 1: Schematic drawing of an alternate splicing construct of the present invention. Alternate splicing of the polypeptide gives rise to two splice products. For splice product 1, the codons coding for the two last amino acids glycin (G) and lysine (K) are incorporated in the mRNA and the polypeptide is secreted from the cell. For splice product 2, the polypeptide does not include the two amino acids G and K, but is extended by a transmembrane region, composed of an optional connecting region, a transmembrane domain and an optional small intracellular domain and the resulting polypeptide is not secreted, but presented on the outer membrane of the cell.

FIG. 2: Staining profiles of CHO-S cells transfected with alternate splicing constructs for the display and secretion of an IgG1 antibody using different transmembrane regions. The analysis was performed the day after transfection. As a negative control, CHO-S cells were transfected with non-splicing constructs coding for the secreted IgG1 antibody (ctrl). The staining performed for the cells transfected with different alternate splicing constructs is presented as a filled histogram and the staining of the control cells transfected with non-splicing constructs coding for secreted IgG1 antibody is included as overlaying black line (A-D). The histograms were used in order to determine the percentage of stained cells (E) and the mean fluorescence of staining (G) of the alternate splicing constructs and the control. Titers of secreted antibody were determined 4 days after transfection using the Octet device (H).

FIG. 3: Staining profiles of CHO-S cells transfected with alternate splicing constructs for the display and secretion of an IgG4 antibody. The analysis was performed the day after transfection. As a negative control, CHO-S cells were transfected with non-splicing constructs coding for the secreted IgG4 antibody (IgG4). The staining performed for the cells transfected with the alternate splicing construct is presented as a filled histogram and the staining of the control cells transfected with non-splicing construct coding for secreted IgG4 antibody is included as overlaying black line (A). The histograms were used in order to determine the percentage of stained cells (B) and the mean fluorescence of staining (D) of the alternate splicing construct and the control. Titers of secreted antibody were determined 4 days after transfection using the Octet device (C).

FIG. 4: Specific detection of a bispecific antibody displayed on the cell membrane using alternate splicing technology. Cells were stained 1 day after transfection. Fc fragments (scFv-Fc and HC) were detected using a PE conjugated goat anti-human Fc gamma specific antibody (A-C, J-L). Kappa light chains were detected using a mouse anti-human kappa LC APC labelled antibody (D-F, M-O). The staining of the scFv-Fc was performed using a FITC-labelled Protein A. The staining performed for the transfected cells was presented as a filled histogram. For the first transfection cocktail (A, D, G) the profile obtained for non-transfected cells was presented as an overlay (black line). For all other experiments (B, C, E, F and H-R) the negative controls (profiles obtained for transfected cells without transmembrane domain) were presented as an overlay (dashed lines).

FIG. 5: Titers of secreted BEAT® and scFv-Fc homodimer molecule 6 days after transient transfection.

FIG. 6: Staining profiles of CHO-S cells transfected with alternate splicing constructs for display and secretion of an IgG1 antibody including different transmembrane regions and different modifications of the expression construct. The analysis was performed the day after transfection. As a negative control, CHO-S cells were transfected with non-splicing constructs coding for secreted IgG1 antibody (Control).

FIG. 7: Normalized expression levels of different alternate splicing constructs for display and secretion of an IgG1 antibody transfected in CHO-S cells. The supernatant was analysed using the Octet QK device and protein A bioprobes (standard HC: Control without alternate splicing).

FIG. 8: Staining profiles of CHO-S cells transfected with alternate splicing constructs for the display and secretion of an IgG1 antibody using the B7-1 transmembrane region and different Y content of the poly(Y) tract of the splice acceptor. The analysis was performed the day after transfection. As a negative control, CHO-S cells were transfected with non-splicing constructs coding for secreted IgG1 antibody (ctrl). The staining performed for the cells transfected with different alternate splicing constructs is presented as a filled histogram, the staining of cells transfected with the non-splicing construct coding for secreted IgG1 antibody is included as overlaying black line (A-G). The histograms were used in order to determine the percentage of stained cells (H) and the mean fluorescence of staining (J) of the alternate splicing constructs and the control. Titers of secreted antibody were determined 4 days after transfection using the Octet device (I).

FIG. 9: Staining profiles of CHO-S cells transfected with alternate splicing constructs for the display and secretion of an IgG1 antibody using the B7-1 transmembrane region and modification of the splice donor consensus sequence. The analysis was performed the day after transfection. As a negative control, CHO-S cells were transfected with non-splicing constructs coding for secreted IgG1 (ctrl). The staining performed for the cells transfected with different alternate splicing constructs is presented as a filled histogram, the staining of cells transfected with the non-splicing constructs coding for secreted IgG1 antibody is included as overlaying black line (A-C). The histograms were used in order to determine the percentage of stained cells (D) and the mean fluorescence of staining (F) of the alternate splicing constructs and the control. Titers of secreted antibody were determined 4 days after transfection using the Octet device (E).

FIG. 10: Staining profiles of CHO-S cells transfected with alternate splicing constructs for the display and secretion of an IgG1 antibody using the B7-1 transmembrane region with different transmembrane domains of 22-23 hydrophobic amino acids. The analysis was performed the day after transfection. As positive control, the alternate splicing construct coding for secreted and membrane displayed IgG1 with B7-1 transmembrane region including B7-1 transmembrane domain was transfected (filled histogram in (A)) and as a negative control, CHO-S cells were transfected with non-splicing constructs coding for secreted IgG1 antibody (overlaying black line in (A)). The staining performed for the cells transfected with constructs coding for secreted and membrane displayed IgG1 using different transmembrane domains is presented as filled histogram, the staining of cells transfected with the construct using the B7-1 transmembrane region including B7-1 transmembrane domain is included as overlaying black line (B-G). The histograms were used in order to determine the percentage of stained cells (H) and the mean fluorescence of staining (I) of the alternate splicing constructs and the control. Titers of secreted antibody were determined 4 days after transfection using the Octet device (G).

FIG. 11: Staining profiles of CHO-S cells transfected with alternate splicing constructs for the display and secretion of an IgG1 antibody using the B7-1 transmembrane region with different transmembrane domains of 21-24 hydrophobic amino acids containing hydrophobic, polar and charged residues. The analysis was performed the day after transfection. As positive control, the alternate splicing construct coding for secreted and membrane displayed IgG1 with B7-1 transmembrane region including the B7-1 transmembrane domain was transfected (filled histogram in (A)) and as a negative control, CHO-S cells were transfected with non-splicing constructs coding for secreted IgG1 antibody (overlaying black line in (A)). The staining performed for the cells transfected with constructs coding for secreted and membrane displayed IgG1 using different transmembrane domains is presented as filled histogram, the staining of cells transfected with the construct using the B7-1 transmembrane region including the B7-1 transmembrane domain is included as overlaying black line (B-H). The staining performed for the cells transfected with constructs coding for secreted and membrane displayed IgG1 using PTCRA transmembrane domains is presented as filled histogram, the staining of cells transfected with non-splicing constructs coding for secreted IgG1 antibody is included as overlaying black line (I). The histograms were used in order to determine the percentage of stained cells (J) and the mean fluorescence of staining (L) of the alternate splicing constructs and the control. Titers of secreted antibody were determined 4 days after transfection using the Octet device (K).

FIG. 12: Staining profiles of CHO-S cells transfected with alternate splicing constructs for the display and secretion of an IgG1 antibody using the B7-1 transmembrane region including PTCRA transmembrane domain with variation of the hydrophobic residues numbers in PTCRA transmembrane domain. The analysis was performed the day after transfection. As positive control, the alternate splicing construct coding for secreted and membrane displayed IgG1 with B7-1 transmembrane region and the wild-type PTCRA transmembrane domain was transfected (filled histogram in (A)) and as a negative control, CHO-S cells were transfected with non-splicing constructs coding for secreted IgG1 antibody (overlaying black line in (A)). The staining performed for the cells transfected with constructs coding for secreted and membrane displayed IgG1 using different modifications of PTCRA transmembrane domains is presented as filled histogram, the staining of cells transfected with the construct using the B7-1 transmembrane region with the wild-type PTCRA transmembrane domain is included as overlaying black line (B-H). The histograms were used in order to determine the percentage of stained cells (I) and the mean fluorescence of staining (K) of the alternate splicing constructs and the control. Titers of secreted antibody were determined 4 days after transfection using the Octet device (J).

FIG. 13: Staining profiles of CHO-S cells transfected with alternate splicing constructs for the display and secretion of an IgG1 antibody using the B7-1 transmembrane region with different transmembrane domains of 17-19 hydrophobic amino acids containing hydrophobic, polar and charged residues. The analysis was performed the day after transfection. As positive control, the alternate splicing construct coding for secreted and membrane displayed IgG1 with B7-1 transmembrane region including B7-1 transmembrane domain was transfected (filled histogram in (A)) and as a negative control, CHO-S cells were transfected with non-splicing constructs coding for secreted IgG1 antibody (overlaying black line in (A)). The staining performed for the cells transfected with constructs coding for secreted and membrane displayed IgG1 using different transmembrane domains is presented as filled histogram, the staining of cells transfected with the construct using the B7-1 transmembrane region including B7-1 transmembrane domain is included as overlaying black line (B-D). The histograms were used in order to determine the percentage of stained cells (E) and the mean fluorescence of staining (G) of the alternate splicing constructs and the control. Titers of secreted antibody were determined 4 days after transfection using the Octet device (F).

FIG. 14: Staining profiles of CHO-S cells transfected with alternate splicing constructs for the display and secretion of an IgG1 antibody using the B7-1 transmembrane region with different transmembrane domains of 26-27 hydrophobic amino acids containing hydrophobic, polar and charged residues. The analysis was performed the day after transfection. As positive control, the alternate splicing construct coding for secreted and membrane displayed IgG1 with B7-1 transmembrane region including the B7-1 transmembrane domain was transfected (filled histogram in (A)) and as a negative control, CHO-S cells were transfected with non-splicing constructs coding for secreted IgG1 antibody (overlaying black line in (A)). The staining performed for the cells transfected with constructs coding for secreted and membrane displayed IgG1 using different transmembrane domains is presented as filled histogram, the staining of cells transfected with the construct using the B7-1 transmembrane region including the B7-1 transmembrane domain is included as overlaying black line (B-E). The histograms were used in order to determine the percentage of stained cells (F) and the mean fluorescence of staining (H) of the alternate splicing constructs and the control. Titers of secreted antibody were determined 4 days after transfection using the Octet device (G).

FIG. 15: Staining profiles of CHO-S cells transfected with alternate splicing constructs for the display and secretion of an IgG1 antibody using the B7-1 transmembrane region with shortened or elongated B7-1 transmembrane domains. The analysis was performed the day after transfection. As positive control, the alternate splicing construct coding for secreted and membrane displayed IgG1 with B7-1 transmembrane region including the wild-type B7-1 transmembrane domain was transfected (filled histogram in (A)) and as a negative control, CHO-S cells were transfected with non-splicing constructs coding for secreted IgG1 antibody (overlaying black line in (A)). The staining performed for the cells transfected with constructs coding for secreted and membrane displayed IgG1 using different transmembrane domains is presented as filled histogram, the staining of cells transfected with the construct using the B7-1 transmembrane region including the B7-1 wild-type transmembrane domain is included as overlaying black line (B-E). The histograms were used in order to determine the percentage of stained cells (F) and the mean fluorescence of staining (H) of the alternate splicing constructs and the control. Titers of secreted antibody were determined 4 days after transient expression using the Octet device (G).

FIG. 16: Staining profiles of CHO-S cells transfected with alternate splicing constructs for the display and secretion of an IgG1 antibody using the B7-1 transmembrane region with different cytosolic tails. The analysis was performed the day after transfection. As positive control, the alternate splicing construct coding for secreted and membrane displayed IgG1 with the B7-1 transmembrane region including the B7-1 cytosolic tail was transfected (filled histogram in (A)) and as a negative control, CHO-S cells were transfected with non-splicing constructs coding for secreted IgG1 antibody (overlaying black line in (A)). The staining performed for the cells transfected with constructs coding for secreted and membrane displayed IgG1 using different cytosolic tails is presented as filled histogram, the staining of cells transfected with the construct using the B7-1 transmembrane region including the B7-1 cytosolic tail is included as overlaying black line (B-N). The histograms were used in order to determine the percentage of stained cells (O) and the mean fluorescence of staining (Q) of the alternate splicing constructs and the control. Titers of secreted antibody were determined 4 days after transfection using the Octet device (P).

FIG. 17:

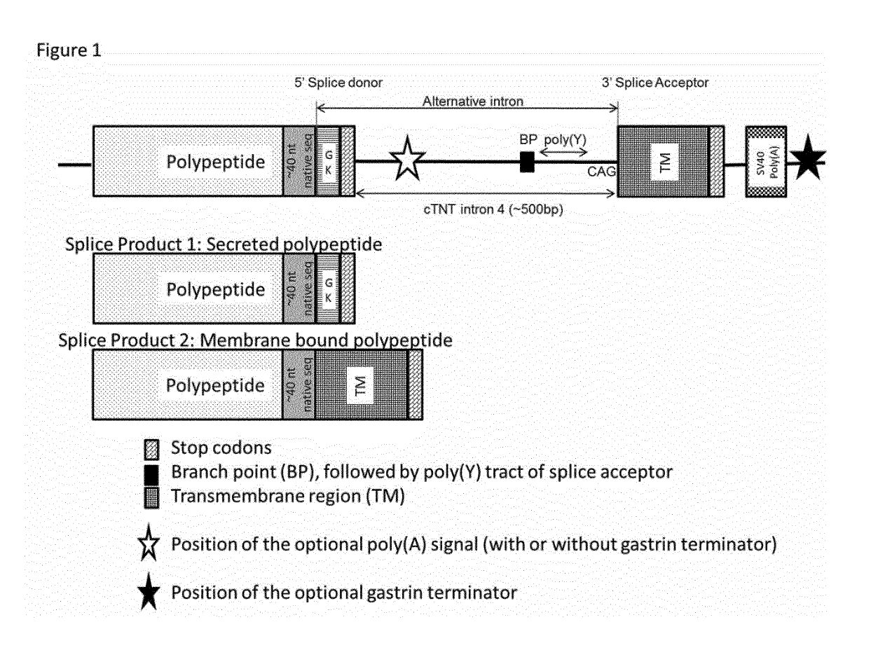

Staining profiles of CHO-S cells transfected with alternate splicing constructs for the display and secretion of an IgG1 antibody using the M1M2 transmembrane region with different cytosolic tails. The analysis was performed the day after transfection. As positive control, the alternate splicing construct coding for secreted and membrane displayed IgG1 with M1M2 transmembrane region including the M1M2 cytosolic tail was transfected (filled histogram in (A)) and as a negative control, CHO-S cells were transfected with non-splicing constructs coding for secreted IgG1 antibody (overlaying black line in (A)). The staining performed for the cells transfected with constructs coding for secreted and membrane displayed IgG1 using the M1M2 transmembrane region with the B7-1 cytosolic tail is presented as filled histogram, the staining of cells transfected with the construct using the M1M2 transmembrane region including the M1M2 cytosolic tail is included as overlaying black line (B). The histograms were used in order to determine the percentage of stained cells (C) and the mean fluorescence of staining (E) of the alternate splicing constructs and the control. Titers of secreted antibody were determined 4 days after transfection using the Octet device (D).

FIG. 18: Assembly variants of the three subunits of the BEAT bispecific antibody.

FIG. 19: Example of a dot plot obtained after dual kappa light chain (LC) and Fc fragment staining of a selected stable cell pool. The potential molecules detected by the staining on the cell membrane are displayed on the right. Fc fragment were detected using a PE conjugated goat anti-human Fc gamma specific antibody. Kappa light chains were detected using a mouse anti-human kappa LC APC labelled antibody.

FIG. 20: Correlation between surface staining and secretion profiles of stable pools transfected with alternate splicing vectors. The percentage of Q6* and Q7* plotted refer to 100% being the entire producing cell population (excluding Q8).

FIG. 21: Dot plot obtained after surface staining of a stable cell pool prior cell sorting. The staining uses soluble Target1 and 2 for the detection of the binders displayed on the cell surface. The potential molecules detected by the staining on the cell membrane are indicated in the corresponding quadrants.

FIG. 22: Correlation between the fraction of BEAT® molecule detected in the supernatants of a 14 days fed-batch and the fraction of cells displaying a BEAT® phenotype on their cell surface.

FIG. 23: Example of the measured surface Fc profile obtained for a selected stable cell pool transfected with the alternate splicing constructs. IgG present on the cell membrane were detected using a PE conjugated goat anti-human Fc gamma specific antibody. Living cells were gated in a FSC vs SSC dot plot (g1 in A) to display the fluorescence distribution of the tested alternate splicing pools (filled histogram, B). The staining of an IgG secreting clone lacking the alternate splicing construct was used a negative control and is presented as an overlay (dashed line).

FIG. 24: Correlation between surface staining intensity and expression level of secreting cells assayed during a batch process: correlation between surface intensity and IgG titer on day 1 of a supplemented batch (A) and correlation between the surface intensity on day 1 and the qP measured during the exponential phase (day 1-3) (B)

FIG. 25: Surface staining intensity on day 1 is predictive of the accumulated secreted IgG on day 7.

FIG. 26: Selection of high producers according to the IgG surface display. Cells displaying “low”, “medium” and “high” density of IgG on their membrane were selected by flow cytometric cell sorting (A). The distribution of the surface fluorescence was determined two weeks following sorting (B) and the specific productivity of the sorted clones was assessed in a supplemented batch (C).

### DETAILED DESCRIPTION OF THE INVENTION

The present invention provides expression constructs and methods for the cell membrane expression of polypeptides, especially heteromultimeric polypeptides. In particular the protein of interest may be recombinant antibodies or fragments thereof or bispecific antibodies, in host cells using alternate splicing. The level of cell membrane display is indicative of the secretion level of the polypeptide in a recombinant host cell and for heterodimeric antibodies the cell membrane display is also indicative of the secretion profile i.e. heterodimer or homodimer expression.

The term “expression construct” or “construct” as used interchangeably herein includes a polynucleotide sequence encoding a polypeptide to be expressed and sequences controlling its expression such as a promoter and optionally an enhancer sequence, including any combination of cis-acting transcriptional control elements. The sequences controlling the expression of the gene, i.e. its transcription and the translation of the transcription product, are commonly referred to as regulatory unit. Most parts of the regulatory unit are located upstream of coding sequence of the gene and are operably linked thereto. The expression construct may also contain a downstream 3′ untranslated region comprising a poly(A) site.

The regulatory unit of the invention is either operably linked to the gene to be expressed, i.e. transcription unit, or is separated therefrom by intervening DNA such as for example by the 5′-untranslated region (5′UTR) of the heterologous gene. Preferably the expression construct is flanked by one or more suitable restriction sites in order to enable the insertion of the expression construct into a vector and/or its excision from a vector. Thus, the expression construct according to the present invention can be used for the construction of an expression vector, in particular a mammalian expression vector.

The term “polynucleotide sequence encoding a polypeptide” as used herein includes DNA coding for a gene, preferably a heterologous gene expressing the polypeptide.

The terms “heterologous coding sequence”, “heterologous gene sequence”, “heterologous gene”, “recombinant gene” or “gene” are used interchangeably. These terms refer to a DNA sequence that codes for a recombinant polypeptide, in particular a recombinant heterologous protein product that is sought to be expressed in a host cell, preferably in a mammalian cell and harvested for further use. The product of the gene can be a polypeptide, glycopeptide or lipoglycopeptides. The heterologous gene sequence may not naturally be present in the host cell and is derived from an organism of the same or a different species and may be genetically modified. Alternatively the heterologous gene sequence is naturally present in the host cell.

The terms “protein” and “polypeptide” are used interchangeably to include a series of amino acid residues connected to the other by peptide bonds between the alpha-amino and carboxy groups of adjacent residues.

The term “promoter” as used herein defines a regulatory DNA sequence generally located upstream of a gene that mediates the initiation of transcription by directing RNA polymerase to bind to DNA and initiating RNA synthesis. Promoters for use in the invention include, for example, viral, mammalian, insect and yeast promoters that provide for high levels of expression, e.g. the mammalian cytomegalovirus or CMV promoter, the SV40 promoter, or any promoter known in the art suitable for expression in eukaryotic cells. Promoters particularly suitable for use in an expression construct of the present invention can be selected from the list consisting of: SV40 promoter, human tk promoter, MPSV promoter, mouse CMV, human CMV, rat CMV, human EFlalpha, Chinese hamster EFlalpha, human GAPDH, hybrid promoters including MYC, HYK and CX promoter, synthetic promoters based on rearrangement of transcription factor binding sites. A particular preferred promoter of the present invention is the mouse CMV promoter.

The term “5′ untranslated region (5′UTR)” refers to an untranslated segment in the 5′ terminus of the pre-mRNA or mature mRNA. On mature mRNA, the 5′UTR typically harbours on its 5′ end a 7-methylguanosine cap and is involved in many processes such as splicing, polyadenylation, mRNA export towards the cytoplasm, identification of the 5′ end of the mRNA by the translational machinery and protection of the mRNAs against degradation (Cowling V H (2010) Biochem J, 425: 295-302).

The term “intron” refers to a segment of nucleic acid non-coding sequence that is transcribed and is present in the pre-mRNA but is excised by the splicing machinery based on the sequences of the splice donor site and splice acceptor site, respectively at the 5′ and 3′ ends of the intron, and therefore not present in the mature mRNA transcript. Typically introns have an internal site, called the branch point, located between 20 and 50 nucleotides upstream of the 3′ splice site. The length of the intron used in the present invention may be between 50 and 10000 nucleotides long. A shortened intron may comprise 50 or more nucleotides. A full length intron may comprise more than 10000 nucleotides. Introns suitable for use in an expression construct of the present invention can be selected from the list consisting of: synthetic or artificial introns; or naturally occurring introns such as -globin/IgG chimeric intron, -globin intron, IgG intron, mouse CMV first intron, rat CMV first intron, human CMV first intron, Ig variable region intron and splice acceptor site (Bothwell et al., (1981) Cell, 24: 625-637; U.S. Pat. No. 5,024,939), introns of the chicken TNT gene and the first intron of EFlalpha or modified versions thereof.

The intron used in the present invention may have splice acceptor and splice donors sites from different introns, e.g. the intron may comprise a splice donor site from a human IgG intron e.g. the splice donor site of the ighg1 gene and a splice acceptor site from the chicken cTNT intron 4.

In a preferred embodiment the intron comprises a splice donor site from a human IgG intron and the splice acceptor site from the chicken cTNT intron 4. More preferred are introns comprising the splice donor site from a human IgG intron and a splice acceptor selected from the group consisting of chicken TNT intron 4 (SEQ ID NO: 69) and constructs derived from chicken TNT intron 4 (SEQ ID NOs: 70-78).

The term “exon” refers to a segment of nucleic acid sequence that is transcribed into mRNA and maintained in the mature mRNA after splicing.

The term “splice site” refers to specific nucleic acid sequences that are capable of being recognized by the splicing machinery of a eukaryotic cell as suitable for being cut and/or ligated to a corresponding splice site. Splice sites allow for the excision of introns present in a pre-mRNA transcript. Typically the 5′ portion of the splice site is referred to as the splice donor site and the 3′ corresponding splice site is referred to as the splice acceptor site. The term splice site includes, for example, naturally occurring splice sites, engineered splice sites, for example, synthetic splice sites, canonical or consensus splice sites, and/or non-canonical splice sites, for example, cryptic splice sites. Splice donor or acceptor sites suitable for use in an expression construct of the present invention can be selected from the list consisting of: Splice donor and acceptor from -globin/IgG chimeric intron, splice donor and acceptor from -globin intron, splice donor and acceptor from IgG intron, splice donor and acceptor from mouse CMV first intron, splice donor and acceptor from rat CMV first intron, splice donor and acceptor from human CMV first intron, splice donor and acceptor from Ig variable region intron and splice acceptor sequence (Bothwell et al., (1981) Cell, 24: 625-637; U.S. Pat. No. 5,024,939), splice donor and acceptor from introns of the chicken TNT gene and splice donor and acceptor from the first intron of the EFlalpha gene or fusion constructs thereof. In a preferred embodiment the splice donor site is from the human IgG intron and the splice acceptor site is selected from the group consisting of chicken TNT intron 4 (SEQ ID NO: 69) and constructs derived from chicken TNT intron 4 (SEQ ID NOs: 70-78).

The term consensus sequence of the splice donor region as used herein refers to the sequence (C/A)AGGU(A/G)AGU (underlined sequences are part of the intron) SEQ ID NO: 345 as described in “Molecular Biology of the Cell” (Alberts et al., Garland Publishing, New York 1995).

The term consensus sequence of the splice acceptor region as used herein refers to the sequence CTRAYY - - - poly(Y) tract - - - NCAGG (underlined sequences are part of the intron; in italics: branch point region) SEQ ID NO: 346 as described in “Molecular Biology of the Cell” (Alberts et al., Garland Science, New York 2002).

The term “stop codon” refers to any one of the three stop codons that signal termination of protein synthesis (TAA (RNA: UAA), TAG (RNA: UAG) and TGA (RNA: UGA). In order to avoid incomplete termination efficiency or “leakiness” of a stop codon, 2,3 or multiple stop codons can be used to signal termination of protein synthesis.

The term “transmembrane region” refers to a polypeptide or protein which is encoded by a nucleic acid sequence and which comprises an optional extracellular part, a transmembrane domain and an optional cytosolic tail. A transmembrane domain is any three-dimensional protein structure which is thermodynamically stable in a membrane and usually comprises a single transmembrane alpha helix of a transmembrane protein, predominantly composed of hydrophobic amino acids. The length of the transmembrane domain is in average 21 amino acids, but might vary between 4 to 48 amino acids (Baeza-Delgado et al., 2012). A transmembrane region comprises an optional N-terminal extracellular connecting stretch of amino acids and a transmembrane domain. In some embodiments, the transmembrane region may further comprise a C-terminal cytoplasmic amino acid stretch or an intracellular domain. The transmembrane region that is found in the human ighg1 gene e.g. is composed of a connecting stretch of amino acids followed by the two domains M1 and M2 (this transmembrane region will be referred to as “M1M2” or “IgG1 transmembrane region”). Transmembrane regions of use in the invention include, but are not limited to, the transmembrane region of the human platelet-derived growth factor receptor (PDGFR) gene (Swissprot entry P16234), human asialoglycoprotein receptor (Swissprot entry P07306), human and murine B7-1 (human: Swissprot entry P33681 and murine: Swissprot entry Q00609), human ICAM-1 (Swissprot entry P05362), human erbb1 (Swissprot entry P00533), human erbb2 (Swissprot entry P04626), human erbb3 (Swissprot entry P21860), human erbb4 (Swissprot entry Q15303), human fibroblast growth factor receptors such as FGFR 1(Swissprot entry P11362),FGFR2 (Swissprot entry P21802), FGFR3 (Swissprot entry P22607), FGFR4 (Swissprot entry P22455), human VEGFR-1 (Swissprot entry P17948), human VEGFR-2 (Swissprot entry P35968), human erythropoietin receptor (Swissprot entry P19235), human PRL-R, prolactin receptor (Swissprot entry P16471), human EphA1, Ephrin type-A receptor 1 (Swissprot entry P21709), human insulin (Swissprot entry P06213), Insulin-like growth factor 1 receptor (IGFR1, Swissprot entry P08069, SEQ ID NO: 181), human receptor-like protein tyrosine phosphatases (Swissprot entries Q12913, P23471, P23467, P18433, P23470, P23469, P23468), human neuropilin (Swissprot entry P014786), human major histocompatibility complex class II (alpha and beta chains), human integrins (alpha and beta families), human Syndecans, human Myelin protein, human cadherins, human synaptobrevin-2 (Swissprot entry P63027), human glycophorin-A (GpA, Swissprot entry P02724, SEQ ID NO: 185), human Bnip3 (Swissprot entry Q12983), human APP (Swissprot entry P05067), amyloid precursor protein (Swissprot entry P0DJI8), human T-cell receptor alpha gene (PTCRA, Swissprot entry PQ6ISU1, SEQ ID NO: 186) and T-cell receptor beta, CD3 gamma (Swissprot entry P09693), CD3 delta (Swissprot entry P04234), CD3 zeta (Swissprot entry P20963), and CD3 epsilon (CD3E, Swissprot entry P07766, SEQ ID NO: 197), human Serine/threonine-protein kinase receptor R3 (ACVL1, Swissprot entry P37023, SEQ ID NO: 174), human Anthrax toxin receptor 2 (ANTR2, Swissprot entry P58335, SEQ ID NO: 175), human T-cell surface glycoprotein CD4 (CD4, Swissprot entry P01730, SEQ ID NO: 176), human Receptor-type tyrosine-protein phosphatase mu (PTPRM, Swissprot entry P28827, SEQ ID NO: 177), human Tumor necrosis factor receptor superfamily member 5 (TNR5, Swissprot entry P25942, SEQ ID NO: 178). Human Integrin beta-1 (ITB1, Swissprot entry P05556, SEQ ID NO: 179), human HLA class I histocompatibility antigen, B-7 alpha chain (Swissprot entry P01889, SEQ ID NO: 180), human Thrombomodulin (TRBM, Swissprot entry P07204, SEQ ID NO: 182), human Interleukin-4 receptor subunit alpha (IL4RA, Swissprot entry P24394, SEQ ID NO: 183), human Low-density lipoprotein receptor-related protein 6 (LRP6, Swissprot entry 075581, SEQ ID NO: 184), human High affinity immunoglobulin epsilon receptor subunit alpha (FCERA, Swissprot entry P12319, SEQ ID NO: 194), human Killer cell immunoglobulin-like receptor 2DL2 (KI2L2, Swissprot entry P43627, SEQ ID NO: 195), human Cytokine receptor common subunit beta (IL3RB, Swissprot entry P32927, SEQ ID NO: 196), human Integrin alpha-IIb (ITA2B, Swissprot entry P08514, SEQ ID NO: 198), human T-cell-specific surface glycoprotein CD28 (CD28, Swissprot entry P10747, SEQ ID NO: 199)

In a preferred embodiment the transmembrane region used in the present invention is selected form the group consisting of the human B7-1 transmembrane region, the murine B7-1 transmembrane region, the PDGFR transmembrane region, the human asialoglycoprotein receptor transmembrane region and the erbb-2 transmembrane region. More preferred is the murine B7-1 transmembrane region, most preferred is the murine B7-1 transmembrane region as shown in SEQ ID NO: 66.

An immunoglobulin transmembrane region includes the transmembrane region from the human immunoglobulin genes IGHA1 (NCBI access code: M60193), IGHA2 (NCBI access code: M60194), IGHD (NCBI access code: K02881), IGHE (NCBI access code: X63693), IGHG1 (NCBI access code: X52847), IGHG2 (NCBI access code: AB006775), IGHG3 (NCBI access code: D78345), IGHG4(NCBI access code: AL928742), IGHGP (NCBI access code: X52849), IGHM (NCBI access code: X14940) as well as the transmembrane regions from the murine immunoglobulin genes IGHA1 (NCBI access code:K00691), IGHD (NCBI access code: J00450), IGHE (NCBI access code: X03624, U08933), IGHG1 (NCBI access code:J00454, J00455), IGHG2A (NCBI access code: J00471), IGHG2B (NCBI access code: J00462, D78344), IGHG3 (NCBI access code: X00915, V01526), IGHM (NCBI access code: J00444).

In one embodiment of the invention the transmembrane region used is a non-immunoglobulin transmembrane region which does not comprise transmembrane regions encoded by immunoglobulin genes, in a particular a non-immunoglobulin transmembrane region which does not comprise transmembrane regions encoded by the human immunoglobulin genes IGHA1 (NCBI access code: M60193), IGHA2 (NCBI access code: M60194), IGHD (NCBI access code: K02881), IGHE (NCBI access code: X63693), IGHG1 (NCBI access code: X52847), IGHG2 (NCBI access code: AB006775), IGHG3 (NCBI access code: D78345), IGHG4(NCBI access code: AL928742), IGHGP (NCBI access code: X52849), IGHM (NCBI access code: X14940) and does not comprise transmembrane regions encoded by the murine immunoglobulin genes IGHA1 (NCBI access code:K00691), IGHD (NCBI access code: J00450), IGHE (NCBI access code: X03624, U08933), IGHG1 (NCBI access code:J00454, J00455), IGHG2A (NCBI access code: J00471), IGHG2B (NCBI access code: J00462, D78344), IGHG3 (NCBI access code: X00915, V01526), IGHM (NCBI access code: J00444).

The term “poly(Y) tract” refers to the stretch of nucleic acids found between the branch point of the intron and the intron-exon border. This stretch of nucleic acids has an abundance of pyrimidines (Ys), meaning an abundance of the pyrimidine bases C or T.

The term “3′ untranslated region (3′UTR)” refers to an untranslated segment in the 3′ terminus of the pre-mRNAs or mature mRNAs. On mature mRNAs this region harbours the poly(A) tail and is known to have many roles in mRNA stability, translation initiation and mRNA export (Jia J et al., (2013) Curr Opin Genet Dev, 23(1): 29-34).

The term “enhancer” as used herein defines a nucleotide sequence that acts to potentiate the transcription of genes independent of the identity of the gene, the position of the sequence in relation to the gene, or the orientation of the sequence. The vectors of the present invention optionally include enhancers.

The term “polyadenylation signal” or “poly(A) signal” or “poly(A)” or “poly(A) site” refers to a nucleic acid sequence present in the mRNA transcripts, that allows for the transcripts, when in the presence of the poly(A) polymerase, to be polyadenylated on the polyadenylation site located 10 to 30 bases downstream the poly(A) signal. Many poly(A) signals are known in the art and may be useful in the present invention. Examples include the human variant growth hormone poly(A) signal, the SV40 late poly(A) signal and the bovine growth hormone poly(A) signal.

The terms “functionally linked” and “operably linked” are used interchangeably and refer to a functional relationship between two or more DNA segments, in particular gene sequences to be expressed and those sequences controlling their expression. For example, a promoter and/or enhancer sequence, including any combination of cis-acting transcriptional control elements is operably linked to a coding sequence if it stimulates or modulates the transcription of the coding sequence in an appropriate host cell or other expression system. Promoter regulatory sequences that are operably linked to the transcribed gene sequence are physically contiguous to the transcribed sequence.

“Orientation” refers to the order of nucleotides in a given DNA sequence. For example, an orientation of a DNA sequence in opposite direction in relation to another DNA sequence is one in which the 5′ to 3′ order of the sequence in relation to another sequence is reversed when compared to a point of reference in the DNA from which the sequence was obtained. Such reference points can include the direction of transcription of other specified DNA sequences in the source DNA and/or the origin of replication of replicable vectors containing the sequence.

The term “nucleic acid sequence homology” or “nucleotide sequence homology” as used herein include the percentage of nucleotides in the candidate sequence that are identical with the nucleotide sequence of the comparison sequence after aligning the sequences and introducing gaps, if necessary, to achieve the maximum percent sequence identity. Thus sequence identity can be determined by standard methods that are commonly used to compare the similarity in position of the nucleotides of two nucleotide sequences.

The term “expression vector” as used herein includes an isolated and purified DNA molecule which upon transfection into an appropriate host cell provides for an appropriate expression level of a recombinant gene product within the host cell. In addition to the DNA sequence coding for the recombinant or gene product the expression vector comprises regulatory DNA sequences that are required for an efficient transcription of the DNA coding sequence into mRNA and for an efficient translation of the mRNAs into proteins in the host cell line.

The terms “host cell” or “host cell line” as used herein include any cells, in particular mammalian cells, which are capable of growing in culture and expressing a desired recombinant product protein.

Recombinant polypeptides and proteins can be produced in various expression systems such as prokaryotic (e.g. E. coli), eukaryotic (e.g. yeast, insect, vertebrate, mammalian), and in vitro expression systems. Most commonly used methods for the large-scale production of protein-based biologics rely on the introduction of genetic material into host cells by transfection of DNA vectors. Transient expression of polypeptides can be achieved with transient transfection of host cells. Integration of vector DNA into the host cell genome results in a cell line that is stably transfected and propagation of such a stable cell line can be used for the large-scale production of polypeptides and proteins.

Methods for producing multiple polypeptides in a eukaryotic cell by means of alternate splicing are described in WO2005/089285. Two different expression cassettes are under control of a single promoter wherein the expression cassettes have splice sites which allow for their alternative splicing and expression as two or more independent gene products at a desired ratio.

The present inventors have found however, that such an approach is limited for the expression of a soluble polypeptide variant and a plasma-membrane-bound variant as two entirely independent proteins will be produced and hence, the plasma-membrane-bound variant does not represent the desired product quality of the soluble polypeptide variant.

Methods for selecting eukaryotic cells expressing a heterologous protein and expression of a soluble polypeptide variant and a plasma-membrane-bound variant from an alternatively spliceable nucleic acid are described in WO2007/131774. The plasma-membrane-bound variant is connected to the cell expressing it and can be used as a marker to isolate cells that have been successfully transfected.

The present inventors have found however, that such an approach is limited as there is no means to control the ratio of soluble polypeptide expression to membrane displayed polypeptide. Alternate splicing, as mentioned above, is a process by which different variants of a polypeptide can be expressed but since the mechanisms of alternate splicing are highly variable, without any ability to control the splicing ratio, the amounts of polypeptide variants expressed will vary.

A cell expression system whereby a portion of the expressed polypeptide is re-directed to be expressed on the cell membrane will therefore have reduced titres of soluble polypeptide. Whilst cell membrane expression of polypeptides is a useful marker to isolate high expressing clones, without any form of control of the amount of polypeptide expressed on the cell membrane, the titres of soluble polypeptide can be reduced significantly. Furthermore, no two polypeptides express at the same level under the same culture conditions, therefore a method developed for the soluble and cell membrane expression of one polypeptide is unlikely to be suitable for all polypeptides.

In contrast to the alternate splicing approaches described previously, the present applicants have designed an alternate splicing approach for the expression of both soluble and cell membrane displayed polypeptide, in which the components of the expression construct can be modified to optimize the amount of soluble polypeptide expressed whilst still permitting cell membrane expression of the polypeptide at an amount sufficient to enable cell clone selection.

In an exemplary expression construct of the present invention for the expression of an antibody heavy chain, the IgG1 heavy chain constant region contains a weak splice donor upstream of the stop codon terminating the open reading frame of the secreted heavy chain. A splice acceptor site and the DNA sequence coding for a transmembrane region followed by a stop codon and a poly(A) site located 3′ of the stop codon so that the transmembrane region is in frame with the open reading frame of the IgG1 heavy chain after splicing. The resulting alternative open reading frames are terminated by a stop codon and a poly(A) site, respectively. The applicants have found that by modifying this expression construct they can alter the splice ratio and therefore the amount of soluble polypeptide versus membrane displayed polypeptide.

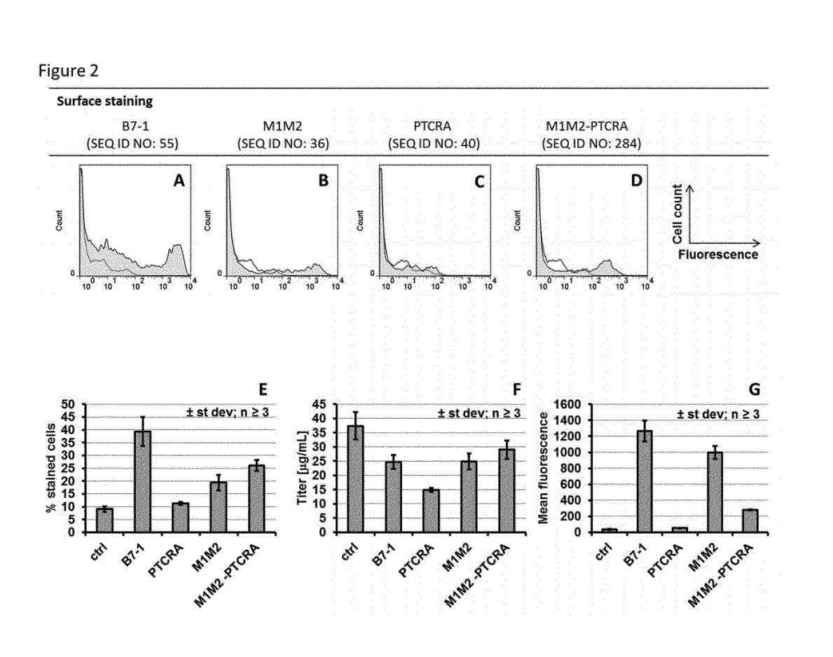

These modifications can include, for example, use of a heterologous transmembrane region. The naturally occurring transmembrane region of IgG1 is composed by a connecting stretch of amino acids and the domains M1 and M2. By replacement of the IgG1 transmembrane region with a transmembrane region which is a non-immunoglobulin transmembrane region e.g. by replacement with the murine B7-1 transmembrane region, the amount of membrane displayed polypeptide can be increased. As is shown in FIG. 2 of Example 2, almost 40% of the transiently transfected cells were positive for membrane displayed IgG1, which is a significant increase over the percentage of cells transfected with a construct comprising the IgG1 transmembrane region (up to 20%). Such a result is surprising as it would be expected that use of the natural IgG1 transmembrane region would be most efficient for the cell membrane display of IgG1, not a heterologous transmembrane region. Preferably, the transmembrane region used in an expression construct of the present invention comprises a transmembrane region selected from the group consisting of the transmembrane region of human platelet-derived growth factor receptor (PDGFR) gene, human asialoglycoprotein receptor, human and murine B7-1, human ICAM-1, human erbb1, human erbb2, human erbb3, human erbb4, human fibroblast growth factor receptors such as FGFR 1,FGFR2, FGFR3, FGFR4, human VEGFR-1, human VEGFR-2, human erythropoietin receptor, human PRL-R, prolactin receptor, human EphA1, Ephrin type-A receptor 1, human insulin, IGF-1 receptors, human receptor-like protein tyrosine phosphatases, human neuropilin, human major histocompatibility complex class II (alpha and beta chains), human integrins (alpha and beta families), human Syndecans, human Myelin protein, human cadherins, human synaptobrevin-2, human glycophorin-A, human Bnip3, human APP, amyloid precursor protein, human T-cell receptor alpha (PTCRA) and T-cell receptor beta, CD3 gamma, CD3 delta, CD3 zeta, and CD3 epsilon. In a preferred embodiment the transmembrane region used in this invention is selected form the group consisting of the human B7-1 transmembrane region, the murine B7-1 transmembrane region, the PDGFR transmembrane region, the human asialoglycoprotein receptor transmembrane region and the erbb-2 transmembrane region. More preferred is the murine B7-1 transmembrane region, most preferred is the murine B7-1 transmembrane region as shown in SEQ ID NO: 66.

Further modifications to alter the splice ratio of the amount of secreted polypeptide versus membrane displayed polypeptide include altering the number of pyrimidine bases in a polypyrimidine (poly(Y)) tract upstream of the exon coding for the transmembrane region and/or modifying the splice donor and acceptor consensus sequences. Decreasing the number of pyrimidine bases (Ys) has been shown in Example 3 to cause a shift in the splice ratio towards secreted protein. If no pyrimidine bases are included in the construct, there is no surface expression of the protein of interest. Such a mechanism to shift the splice ratio away from cell membrane expression could be useful for a protein which is expressed strongly on the cell membrane but shows weaker soluble expression. The number of pyrimidine bases in the poly(Y) tract may comprise between 0 and 30 bases. Preferably, the poly(Y) tract comprises 20 pyrimidine bases or less, more preferably 15 bases or less, even more preferably 10 bases or less.

Since an increase in the amount of membrane displayed protein can have a negative impact on the titres of soluble polypeptide, the inventors have developed modifications to the expression construct that can increase soluble polypeptide expression in the host cell without impacting on the amount of membrane displayed polypeptide. The addition of a poly(A) site in the intron was found to increase soluble polypeptide titre by a maximum of 50% (for the M1M2 transmembrane region) while maintaining a significant level of cell membrane display. In a further embodiment of the present invention, a poly(A) site is located in the intron of the expression construct.

In an aspect of the present invention, the expression construct is suitable for polypeptide multimers and proteins for example antibodies or fragments thereof or bispecific antibodies or fragments thereof.

The term “antibody” as referred to herein includes whole antibodies and any antigen binding fragments or single chains thereof. An “antibody” refers to a glycoprotein comprising at least two heavy (H) chains and two light (L) chains inter-connected by disulfide bonds, or an antigen binding fragment thereof. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain, CL. The VH and VL regions can be further subdivided into regions of hypervariability, termed complementarity determining regions (CDR) which are hypervariable in sequence and/or involved in antigen recognition and/or usually form structurally defined loops, interspersed with regions that are more conserved, termed framework regions (FR or FW). Each VH and VL is composed of three CDRs and four FWs, arranged from amino-terminus to carboxy-terminus in the following order: FW1, CDR1, FW2, CDR2, FW3, CDR3, FW4. The amino acid sequences of FW1, FW2, FW3, and FW4 all together constitute the “non-CDR region” or “non-extended CDR region” of VH or VL as referred to herein.

The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system.

Antibodies are grouped into classes, also referred to as isotypes, as determined genetically by the constant region. Human constant light chains are classified as kappa (C_{κ}) and lambda (C) light chains. Heavy chains are classified as mu (μ), delta (δ), gamma (γ), alpha (α), or epsilon (ε), and define the antibody's isotype as IgM, IgD, IgG, IgA, and IgE, respectively. The IgG class is the most commonly used for therapeutic purposes. In humans this class comprises subclasses IgG1, IgG2, IgG3 and IgG4.

The term “Fab” or “Fab region” as used herein includes the polypeptides that comprise the VH, CH1, VL, and CL immunoglobulin domains. Fab may refer to this region in isolation, or this region in the context of a full length antibody or antibody fragment.

The term “Fc” or “Fc region”, as used herein includes the polypeptide comprising the constant region of an antibody excluding the first constant region immunoglobulin domain. Thus Fc refers to the last two constant region immunoglobulin domains of IgA, IgD, and IgG, and the last three constant region immunoglobulin domains of IgE and IgM, and the flexible hinge N-terminal to these domains. For IgA and IgM, Fc may include the J chain. For IgG, Fc comprises immunoglobulin domains C gamma 2 and C gamma 3 (C2 and C3) and the hinge between C gamma 1 (C1) and C gamma 2 (C2). Although the boundaries of the Fc region may vary, the human IgG heavy chain Fc region is usually defined to comprise residues C226 or P230 to its carboxyl-terminus, wherein the numbering is according to the EU numbering system. For human IgG1 the Fc region is herein defined to comprise residue P232 to its carboxyl-terminus, wherein the numbering is according to the EU numbering system (Edelman G M et al., (1969) Proc Natl Acad Sci USA, 63(1): 78-85). Fc may refer to this region in isolation or this region in the context of an Fc polypeptide, for example an antibody.

The term “full length antibody” as used herein includes the structure that constitutes the natural biological form of an antibody, including variable and constant regions. For example, in most mammals, including humans and mice, the full length antibody of the IgG class is a tetramer and consists of two identical pairs of two immunoglobulin chains, each pair having one light and one heavy chain, each light chain comprising immunoglobulin domains VL and CL, and each heavy chain comprising immunoglobulin domains VH, CH1 (C1), CH2 (C2), and CH3 (C3). In some mammals, for ex ample in camels and llamas, IgG antibodies may consist of only two heavy chains, each heavy chain comprising a variable domain attached to the Fc region.

Antibody fragments include, but are not limited to, (i) the Fab fragment consisting of VL, VH, CL and CH1 domains, including Fab and Fab -SH, (ii) the Fd fragment consisting of the VH and CH1 domains, (iii) the Fv fragment consisting of the VL and VH domains of a single antibody; (iv) the dAb fragment (Ward E S et al., (1989) Nature, 341: 544-546) which consists of a single variable, (v) F(ab′)₂ fragments, a bivalent fragment comprising two linked Fab fragments (vi) single chain Fv molecules (scFv), wherein a VH domain and a VL domain are linked by a peptide linker which allows the two domains to associate to form an antigen binding site (Bird R E et al., (1988) Science 242: 423-426; Huston J S et al., (1988) Proc. Natl. Acad. Sci. USA, 85: 5879-83), (vii) bispecific single chain Fv dimers (PCT/US92/09965), (viii) “diabodies” or “triabodies”, multivalent or multispecific fragments constructed by gene fusion (Tomlinson I & Hollinger P (2000) Methods Enzymol. 326: 461-79; WO94/13804; Holliger P et al., (1993) Proc Natl Acad Sci USA, 90: 6444-48) and (ix) scFv genetically fused to the same or a different antibody (Coloma M J & Morrison S L (1997) Nature Biotech, 15(2): 159-163).

Antibodies and fragment thereof that can be expressed by an expression construct as described herein may bind to an antigen selected from the group consisting of: AXL, Bcl2, HER2, HERS, EGF, EGFR, VEGF, VEGFR, IGFR, PD-1, PD-1L, BTLA, CTLA-4, GITR, mTOR, CS1, CD3, CD16, CD16a, CD19, CD20, CD22, CD25, CD27, CD28, CD30, CD32b, CD33, CD38, CD40, CD52, CD64, CD79, CD89, CD137, CD138, CA125, cMet, CCR6, MUCI, PEM antigen, Ep-CAM, EphA2, 17-1a, CEA, AFP, HLA class II, HLA-DR, HSG, IgE, IL-12, IL-17a, IL-18, IL-23, IL-1alpha, IL-1beta, GD2-ganglioside, MCSP, NG2, SK-I antigen, Lag3, PAR2, PDGFR, PSMA, Tim3, TF, CTLA4, TL1A, TIGIT, SIRPa, ICOS, Treml2, NCR3, HVEM, OX40, VLA-2 and 4-1BB.

Bispecific or heterodimeric antibodies have been available in the art for many years. However the generation of such antibodies is often associated with the presence of mispaired by-products, which reduces significantly the production yield of the desired bispecific antibody and requires sophisticated purification procedures to achieve product homogeneity. The mispairing of immunoglobulin heavy chains can be reduced by using several rational design strategies, most of which engineer the antibody heavy chains for heterodimerisation via the design of man-made complementary heterodimeric interfaces between the two subunits of the CH3 domain homodimer. The first report of an engineered CH3 heterodimeric domain pair was made by Carter et al. describing a “protuberance-into-cavity” approach for generating a hetero-dimeric Fc moiety (U.S. Pat. No. 5,807,706; Inobs-into-holes′; Merchant A M et al., (1998) Nat Biotechnol, 16(7):677-81). Alternative designs have been recently developed and involved either the design of a new CH3 module pair by modifying the core composition of the modules as described in WO2007110205 or the design of complementary salt bridges between modules as described in WO2007147901 or WO2009089004. The disadvantage of the CH3 engineering strategies is that these techniques still result in the production of a significant amount of undesirable homo-dimers. A more preferred technique for generating bispecific antibodies in which predominantly heterodimers are produced is described in PCT Publication No: WO2012/131555. Bispecific antibodies can be generated to a number of targets, for example, a target located on tumour cells and/or a target located on effector cells. Preferably, a bispecific antibody can bind to two targets selected from the group consisting of: AXL, Bcl2, HER2, HERS, EGF, EGFR, VEGF, VEGFR, IGFR, PD-1, PD-1L, BTLA, CTLA-4, GITR, mTOR, CS1, CD3, CD16, CD16a, CD19, CD20, CD22, CD25, CD27, CD28, CD30, CD32b, CD33, CD38, CD40, CD52, CD64, CD79, CD89, CD137, CD138, CA125, cMet, CCR6, MUCI, PEM antigen, Ep-CAM, EphA2, 17-1a, CEA, AFP, HLA class II, HLA-DR, HSG, IgE, IL-12, IL-17a, IL-18, IL-23, IL-1alpha, IL-1beta, GD2-ganglioside, MCSP, NG2, SK-I antigen, Lag3, PAR2, PDGFR, PSMA, Tim3, TF, CTLA4, TL1A, TIGIT, SIRPa, ICOS, Treml2, NCR3, HVEM, OX40, VLA-2 and 4-1BB.

In a further aspect, the present invention provides a host cell comprising an expression construct or an expression vector as described supra. The host cell can be a human or non-human cell. Usually the host cell is selected from the group consisting of a mammalian cell, an insect cell and a yeast cell. Preferred host cells are mammalian cells. Preferred examples of mammalian host cells include, without being restricted to, Human embryonic kidney cells (Graham F L et al., (1977) J. Gen. Virol. 36: 59-74), MRC5 human fibroblasts, 983M human melanoma cells, MDCK canine kidney cells, RF cultured rat lung fibroblasts isolated from Sprague-Dawley rats, B16BL6 murine melanoma cells, P815 murine mastocytoma cells, MT1 A2 murine mammary adenocarcinoma cells, PER:C6 cells (Leiden, Netherlands) and Chinese hamster ovary (CHO) cells or cell lines (Puck T T et al., (1958), J. Exp. Med. 108: 945-955).

In a particular preferred embodiment the host cell is a Chinese hamster ovary (CHO) cell or cell line. Suitable CHO cell lines include e.g. CHO-S(Invitrogen, Carlsbad, Calif., USA), CHO K1 (ATCC CCL-61), CHO pro3-, CHO DG44, CHO P12 or the dhfr− CHO cell line DUK-BII (Urlaub G & Chasin L A (1980) PNAS 77(7): 4216-4220), DUXBI1 (Simonsen C C & Levinson A D (1983) PNAS 80(9): 2495-2499), or CHO-K1SV (Lonza, Basel, Switzerland).

In a further aspect, the present disclosure provides a method for the selection of a host cell expressing a polypeptide of interest comprising transfecting a host cell with the expression construct or an expression vector as described supra, culturing the host cell, detecting cell membrane expression of the polypeptide of interest and selecting the host cell clone with the desired cell membrane expression. In a further step the polypeptide of interest can be recovered from the host cell. The polypeptide is preferably a heterologous, more preferably a human polypeptide. As is shown in Examples, the level of cell membrane expression of the polypeptide of interest is directly proportional to the level of soluble polypeptide expressed. Therefore this enables the quantitative selection of a host cell clone expressing high levels of the polypeptide of interest.

In a preferred embodiment, the present invention provides an in vitro method for the selection of a host cell expressing a multimeric protein, preferable a heterodimeric antibody by transfecting a host cell with the expression constructs or expression vectors as described supra, culturing the host cell, detecting cell membrane expression of the multimeric protein of interest and selecting the host cell clone with the desired cell membrane expression. In a further step the multimeric protein of interest, for example a heterodimeric antibody can be recovered from the host cell. As is shown in the Examples, a host cell which displays more heteromodimeric antibody over unwanted homodimeric antibody species can be easily selected for i.e. allowing for qualitative selection of a host cell clone.

For transfecting the expression construct or the expression vector into a host cell according to the present invention any transfection technique such as those well-known in the art, e.g. electroporation, calcium phosphate co-precipitation, cationic polymer mediated transfection, lipofection, can be employed if appropriate for a given host cell type. It is to be noted that the host cell transfected with the expression construct or the expression vector of the present invention is to be construed as being a transiently or stably transfected cell line. Thus, according to the present invention the present expression construct or the expression vector can be maintained episomally i.e. transiently transfected or can be stably integrated in the genome of the host cell i.e. stably transfected.

A transient transfection is characterised by non-appliance of any selection pressure for a vector borne selection marker. In transient expression experiments which commonly last two to up to ten days post transfection, the transfected expression construct or expression vector are maintained as episomal elements and are not yet integrated into the genome. That is the transfected DNA does not usually integrate into the host cell genome. The host cells tend to lose the transfected DNA and the cells having lost the transfected DNA tend to overgrow transfected cells in the population upon culture of the transiently transfected cell pool. Therefore expression is strongest in the period immediately following transfection and decreases with time. Preferably, a transient transfectant according to the present invention is understood as a cell that is maintained in cell culture in the absence of selection pressure up to a time of two to ten days post transfection.

In a preferred embodiment of the invention the host cell e.g. the CHO host cell is stably transfected with the expression construct or the expression vector of the present invention. Stable transfection means that newly introduced foreign DNA such as vector DNA is becoming incorporated into genomic DNA, usually by random, non-homologous recombination events. The copy number of the vector DNA and concomitantly the amount of the gene product can be increased by selecting cell lines in which the vector sequences have been amplified after integration into the DNA of the host cell. Therefore, it is possible that such stable integration gives rise, upon exposure to further increases in selection pressure for gene amplification, to double minute chromosomes in CHO cells. Furthermore, a stable transfection may result in loss of vector sequence parts not directly related to expression of the recombinant gene product, such as e.g. bacterial copy number control regions rendered superfluous upon genomic integration. Therefore, a transfected host cell has integrated at least part or different parts of the expression construct or the expression vector into the genome.

In a further aspect, the present disclosure provides the use of the expression construct or an expression vector as described supra for the expression of a heterologous polypeptide from a mammalian host cell, in particular the use of the expression construct or an expression vector as described supra for the in vitro expression of a heterologous polypeptide from a mammalian host cell. In a preferred embodiment the expression constructs or expression vectors as described supra are used for the in vitro expression of a heterodimeric antibody from a mammalian host cell.

An expression construct as described in the present invention can be used in a method for selecting a recombinant host cell expressing a polypeptide or protein of interest. Preferably, the protein of interest is an antibody. The method comprises:
(i) co-transfecting a host cell with:
   (a) an expression construct comprising in a 5′ to 3′ direction:
   a promoter;
   an exon encoding an antibody heavy chain;
   a splice donor site, an intron and a splice acceptor site, wherein a first stop codon is located between the splice donor site and the splice acceptor site within said intron;
   a second exon encoding a transmembrane region which is selected from a modified immunoglobulin transmembrane region or a modified or unmodified non-immunoglobulin transmembrane region;
   a second stop codon; and
   a poly(A) site,
   (b) an expression construct encoding an antibody light chain,
(ii) culturing the transfected host cell under conditions suitable for expression of the antibody;
(iii) detecting cell membrane expression of the antibody; and
(iv) selecting the host cell displaying the antibody on the surface of the cell membrane.

Furthermore, the above detailed method can be used for the selection of a recombinant host cell expressing a bispecific antibody whereby the host cell is co-transfected with a third expression construct encoding a scFv-Fc or a third expression construct encoding a scFv-Fc with splicing of a non-immunoglobulin transmembrane region, such as a murine B7-1 transmembrane region.

Selection of the desired host cell can be made according to methods known in the art. Such methods include, fluorescence, ELISA, Western blotting, SDS polyacrylamide gel electrophoresis, radioimmunoassay or by using antibodies or specific molecules such as aptamers that recognise and bind to the protein of interest. Preferably, the protein of interest displayed on the membrane of the host cell is detected by a fluorescent probe, or linked to a fluorescent label, therefore permitting selection using fluorescence activated cell sorting or FACS.

Expression and recovering of the protein can be carried out according to methods known to the person skilled in the art.

In a further aspect, the present disclosure provides the use of the expression construct or the expression vector as described supra for the preparation of a medicament for the treatment of a disorder.

In a further aspect, the present disclosure provides the expression construct or the expression vector as described supra for use as a medicament for the treatment of a disorder.

In a further aspect, the present disclosure provides the expression construct or the expression vector as described supra for use in gene therapy.

### EXAMPLES

### Example 1: Cloning of the Vectors

### Introduction

The alternate splicing constructs that were prepared comprised the splice donor sequence of the human ighg1 gene. The introns (including the splice acceptor site) were derived from the chicken cTNT intron 4, whereas the exon was derived from the transmembrane region of the ighg1 gene (in the following referred to as M1M2), the transmembrane region of the T-cell receptor alpha (PTCRA), or the murine B7-1 gene. In several constructs, the murine B7-1 transmembrane domain was replaced by other transmembrane domains, including naturally occurring transmembrane domains and modifications of these. Furthermore, the B7-1 cytosolic tail in the B7-1 transmembrane region was replaced by several other cytosolic tails with or without an ER exportation signal.

For the expression of a full length antibody of IgG1 or IgG4 format, the simultaneous expression of heavy and light chain was necessary. All subunits were expressed using separate plasmids using a co-transfection strategy. The alternate splicing construct was used for the expression of the IgG1 or IgG4 heavy chain. The light chain of the IgG1 or IgG4 was cloned in the same expression vector backbone, but without alternate splicing.

For expression of the in-house generated bispecific antibody formats (BEAT® technology; described in WO2012/131555), three different subunits had to be transfected in cells: heavy chain, light chain and the scFv-Fc. In order to evaluate the best strategy for membrane display of the bispecific antibody format, spliced and alternate splicing constructs were cloned for expression of the heavy chain and the scFv-Fc. The light chain was cloned in the same expression vector backbone, but without alternate splicing. Table 1 summarises all the constructs generated in this example.

TABLE 1
Summary of all constructs generated (*)

| | Plasmid | | |
|---|---|---|---|
| | batch | DNA Sequence | Protein Sequence |
| Plasmid name | number | SEQ ID NO | SEQ ID NO |
| pGLEX41_IgG4-HC | GSB59 | 79 | 126 |
| pGLEX41_HC | GSC314 | 48 | 286 |
| pGLEX41_LC | GSC315 | 294 | 295 |
| pGLEX41_IgG4-LC | GSC3704 | 319 | 320 |
| pGLEX41_HC-AS | GSC3836 | 287 | 288 |
| pGLEX41_HC-cTNTintron4 | GSC3848 | 289 | 288 |
| pGLEX41_HC-I4-M1M2-M1M2-M1M2 | GSC3899 | 36 | 290 |
| pGLEX41_HC-I4(0Y)-M1M2-M1M2-M1M2 | GSC4398 | 37 | 290 |
| pGLEX41_HC-I4(polyA)-M1M2-M1M2-M1M2 | GSC4401 | 38 | 290 |
| pGLEX41_HC-I4-M1M2-M1M2-M1M2-SV40ter | GSC4402 | 39 | 290 |
| pGLEX41_BEAT-HC-I4-M1M2-M1M2-M1M2 | GSC5537 | 50 | 299 |
| pGLEX41_scFv-Fc-I4-M1M2-M1M2-M1M2 | GSC5538 | 52 | 301 |
| pGLEX41_BEAT-LC | GSC5540 | 302 | 303 |
| pGLEX41_BEAT-HC-His | GSC5607 | 296 | 297 |
| pGLEX41_scFv-Fc | GSC5608 | 51 | 300 |
| pGLEX41_BEAT-HC | GSC5644 | 49 | 298 |
| pGLEX41_HC-I4-PTCRA | GSC5854 | 40 | 291 |
| pGLEX41_HC-I4(0Y)-PTCRA | GSC5855 | 41 | 291 |
| pGLEX41_HC-I4-PTCRA-SV40ter | GSC5856 | 43 | 291 |
| pGLEX41_HC-I4(polyA)-PTCRA | GSC5857 | 42 | 291 |
| pGLEX41_HC-I4-M1M2-PTCRA-M1M2 | GSC6030 | 44 | 292 |
| pGLEX41_HC-I4-M1M2-PTCRA-M1M2-SV40ter | GSC6046 | 47 | 292 |
| pGLEX41_HC-I4(polyA)-M1M2-PTCRA-M1M2 | GSC6047 | 46 | 292 |
| pGLEX41_HC-I4(0Y)-M1M2-PTCRA-M1M2 | GSC6048 | 45 | 292 |
| pGLEX41_HC-I4-B7-B7-B7 | GSC7056 | 55 | 293 |
| pGLEX41_HC-I4(0Y)-B7-B7-B7 | GSC7057 | 53 | 293 |
| pGLEX41_HC-I4(polyA)-B7-B7-B7 | GSC7058 | 54 | 293 |
| pGLEX41_HC-I4-B7-B7-B7-SV40ter | GSC7059 | 56 | 293 |
| pGLEX41_HC-I4(5Y)-I4-B7-B7-B7 | GSC9763 | 61 | 293 |
| pGLEX41_HC-SD_CCC-I4-B7-B7-B7 | GSC9764 | 58 | 293 |
| pGLEX41_HC-SD_GGC-I4-B7-B7-B7 | GSC9765 | 57 | 293 |
| pGLEX41_HC-I4(5Y-5)-I4-B7-B7-B7 | GSC9768 | 60 | 293 |
| pGLEX41_HC-I4(3Y)-I4-B7-B7-B7 | GSC9770 | 62 | 293 |
| pGLEX41_HC-I4(9Y)-I4-B7-B7-B7 | GSC9949 | 59 | 293 |
| pGLEX41_HC-I4(15Y-3′)-I4-B7-B7-B7 | GSC9950 | 63 | 293 |
| pGLEX41_scFv-Fc-I4-B7-B7-B7 | GSC10487 | 323 | 324 |
| pGLEX41_BEAT-HC-I4-B7-B7-B7 | GSC10488 | 321 | 322 |
| pGLEX41_HC-I4-B7-PTCRA_K13V-B7 | GSC11203 | 95 | 142 |
| pGLEX41_HC-I4-B7-PTCRA-B7 | GSC11204 | 93 | 140 |
| pGLEX41_HC-I4-B7-PTPMR-B7 | GSC11205 | 84 | 131 |
| pGLEX41_HC-I4-M1M2-PTCRA-M1M2_version 1 | GSC11206 | 284 | 285 |
| pGLEX41_HC-I4-B7-B7-6His | GSC11207 | 112 | 159 |
| pGLEX41_HC-I4-B7-PTCRA_K13V_D18V-B7 | GSC11208 | 99 | 146 |
| pGLEX41_HC-I4-B7-B7-KCFCK | GSC11209 | 113 | 160 |
| pGLEX41_HC-I4-B7-CD4-B7 | GSC11210 | 83 | 130 |
| pGLEX41_HC-I4-B7-PTCRA_D18V-B7 | GSC11211 | 96 | 143 |
| pGLEX41_HC-I4-B7-PTCRA_R8V_K13V-B7 | GSC11212 | 97 | 144 |
| pGLEX41_HC-I4-B7-ACVL1-B7 | GSC11213 | 81 | 128 |
| pGLEX41_HC-I4-B7-ANTR2-B7 | GSC11214 | 82 | 129 |
| pGLEX41_HC-I4-B7-PTCRA_R8V-B7 | GSC11216 | 94 | 141 |
| pGLEX41_HC-I4-B7-TNR5-B7 | GSC11217 | 85 | 132 |
| pGLEX41_IgG4-HC-B7-B7-B7 | GSC11218 | 80 | 127 |
| pGLEX41_HC-I4-B7-PTCRA_R8V_K13V_D18V-B7 | GSC11291 | 100 | 147 |
| pGLEX41_HC-I4-B7-B7(26)-B7 | GSC11292 | 111 | 158 |
| pGLEX41_HC-I4-B7-IGF1R-B7 | GSC11295 | 88 | 135 |
| pGLEX41_HC-I4-B7-1B07-B7 | GSC11296 | 87 | 134 |
| pGLEX41_HC-I4-B7-FCERA-B7 | GSC11297 | 101 | 148 |
| pGLEX41_HC-I4-B7-KI2L2-B7 | GSC11298 | 102 | 149 |
| pGLEX41_HC-I4-B7-M1M2-B7 | GSC11389 | 107 | 154 |
| pGLEX41_HC-I4-B7-TRBM-B7 | GSC11390 | 89 | 136 |
| pGLEX41_HC-I4-B7-IL4RA-B7 | GSC11391 | 90 | 137 |
| pGLEX41_HC-I4-B7-LRP6-B7 | GSC11392 | 91 | 138 |
| pGLEX41_HC-I4-B7-IL3RB-B7 | GSC11393 | 103 | 150 |
| pGLEX41_HC-I4-B7-CD3E-B7 | GSC11394 | 104 | 151 |
| pGLEX41_HC-I4-B7-ITA2B-B7 | GSC11395 | 105 | 152 |
| pGLEX41_HC-I4-B7-CD28-B7 | GSC11396 | 106 | 153 |
| pGLEX41_HC-I4-B7-GpA-B7 | GSC11397 | 92 | 139 |
| pGLEX41_HC-I4-B7-PTCRA_R8V_D18V-B7 | GSC11398 | 98 | 145 |
| pGLEX41_HC-I4-B7-B7-GAT1noER | GSC11483 | 118 | 165 |
| pGLEX41_HC-I4-B7-B7(24)-B7 | GSC11484 | 110 | 157 |
| pGLEX41_HC-I4-B7-B7(18)-B7 | GSC11677 | 108 | 155 |
| pGLEX41_HC-I4-B7-B7-ERGIC53_noER | GSC11678 | 116 | 163 |
| pGLEX41_HC-I4-B7-B7(20)-B7 | GSC11679 | 109 | 156 |
| pGLEX41_HC-I4-B7-ITB1-B7 | GSC11756 | 86 | 133 |
| pGLEX41_HC-I4-B7-B7-M1M2 | GSC11758 | 114 | 161 |
| pGLEX41_HC-I4-B7-B7-GAT1 | GSC11759 | 117 | 164 |
| pGLEX41_HC-I4-B7-B7-AGTR2 | GSC11760 | 119 | 166 |
| pGLEX41_HC-I4-B7-B7-AGTR2_noER | GSC11761 | 120 | 167 |
| pGLEX41_HC-I4-B7-B7-V1BR | GSC11762 | 121 | 168 |
| pGLEX41_HC-I4-B7-B7-VGLG_noER | GSC11763 | 124 | 171 |
| pGLEX41_HC-I4-B7-B7-ERGIC53 | GSC11764 | 115 | 162 |
| pGLEX41_HC-I4-B7-B7-V1BR_noER | GSC11765 | 122 | 169 |
| pGLEX41_HC-I4-B7-B7-VGLG | GSC11766 | 123 | 170 |
| pGLEX41_HC-I4-M1M2-M1M2-B7 | GSC11294 | 125 | 172 |

(*) Sequence listed stretches from the last 5 amino acids of the non-spliced ORF to the end of the expression construct for DNA sequences and from the same amino acids to the end of the fused transmembrane region for protein sequences, except for GSC314 (SEQ ID NO: 48), GSC315 (SEQ ID NO: 294), GSC5607 (SEQ ID NO: 296), GSC5644 (SEQ ID NO: 49), GSC5608 (SEQ ID NO: 51), GSC5540 (SEQ ID NO: 302), GSB59 (SEQ ID NO: 79) (complete ORF sequence).

### Materials and Methods

### LB Culture Plates

500 ml of water were mixed and boiled with 16 g of LB Agar (Invitrogen, Carlsbad, Calif., USA) (1 liter of LB contains 10 g tryptone, 5 g yeast extract and 10 g NaCl). After cooling, the respective antibiotic was added to the solution which was then plated (ampicillin plates at 100 g/ml and kanamycin plates at 50 g/ml).

### Polymerase Chain Reaction (PCR)

All PCRs were performed using 1 l of dNTPs (10 mM for each dNTP; Invitrogen, Carlsbad, Calif., USA), 2 units of Phusion® DNA Polymerase (Finnzymes Oy, Espoo, Finland), 25 nmol of Primer A (Microsynth, Balgach, Switzerland or Operon, Ebersberg, Germany), 25 nmol of Primer B (Microsynth, Balgach, Switzerland or Operon, Ebersberg, Germany), 10 l of 5×HF buffer (7.5 mM MgCl₂, Finnzymes, Espoo, Finland), 1.5 l of Dimethyl sulfoxide (DMSO, Finnzymes, Espoo, Finland) and 1-3 l of the template (10-20 ng) in a 501 final volume. All primers used are listed in Table 2.

The PCRs were started by an initial denaturation at 98° C. for 3 min, followed by 35 cycles of 30 sec denaturation at 98° C., 30 sec annealing at a primer-specific temperature (according to GC content) and 2 min elongation at 72° C. A final elongation at 72° C. for 10 min was performed before cooling and keeping at 4° C.

TABLE 2
Summary of primers used in PCRs. Primers were obtained either from Operon
(Ebersberg, Germany) or from Microsynth (Balgach, Switzerland).

| | | SEQ ID |
|---|---|---|
| Primer name | Primer sequence | NO |
| GlnPr269 | CAGGGGGGCGGAGCCTATGG | 1 |
| GlnPr497 | GCCCCCCTCCCGCGAGGAGATGACCAAGAACCAGGTGTCCCTG | 2 |
| GlnPr498 | TTTCTAGATTCATCACTTGCCGGGGGACAGGCTC | 3 |
| GlnPr501 | TATAAAGCTTCCACCATGGAGAGCCCTGCCC | 4 |
| GlnPr502 | TATATCTAGATTCATCAGCACTCGCCCCGGTTG | 5 |
| GlnPr1285 | CGGAAGAATTCAGCCACAGCTTTAAGGCACCTGGCTAAC | 259 |
| GlnPr1437 | CCAAATCGATTTATCAGCACTCGCCCCGGTTGAAG | 6 |
| GlnPr1452 | ACGTATCGATTCATCACTTGCCGGGGGACAG | 327 |
| GlnPr1487 | CTGCTGCTGTGGCTGCCCGACGGGGTGCACGCTGAGATCGTGCTGACA | 325 |
| | CAGAG | |
| GlnPr1488 | CTGCTGCTGTGGCTGCCCGACGGGGTGCACGCTCAGGTCACACTGAAA | 328 |
| | GAGTC | |
| GlnPr1491 | CCAAATCGATTTATCAGCACTCGCCCCGGTTGAAG | 326 |
| GlnPr1494 | ACGAACTAGTCCACCATGGAAAGCCCAGCCCAGCTGCTGTTCCTGCTG | 260 |
| | CTGCTGTGGCTGCCCGAC | |
| GlnPr1500 | CCGGGATCGATTTATCAGTGATGGTGATGGTGATG | 7 |
| GlnPr1502 | GGCCGATCGATTTATCACTTGCCAGGAGACAG | 8 |
| GlnPr1516 | GATGAAAGCTTGGTAGGTGATCCTCCTG | 9 |
| GlnPr1517 | ATGTATTCGAATATCTCCACCGGTTGCAGCTCTGAGCCAGGGAGGAGG | 10 |
| | GAAG | |
| GlnPr1518 | TAAGCTAGCCCACCATGGAGAGCCCTGC | 11 |
| GlnPr1519 | GAGGCTCTTCTGCGTGTAGTGGTTGTGCAAGGCCTCGTGCATCACGCT | 12 |
| | G | |
| GlnPr1520 | CTACCAAGCTTTCATCATTTACCCGGAGACAGGGAGAGGCTCTTCTGC | 13 |
| | GTG | |
| GlnPr1521 | GGGAGCTGGACGGGCTGTGGACGACCATCACCATCTTCATCACACTCT | 14 |
| | TCCTGTTAAGCGTGTGCTACAGTGCCACCGTCACCTTCTTCAAG | |
| GlnPr1522 | GTAGTCGGGGATGATGGTCTGCTTCAGGTCCACCACCGAGGAGAAGA | 15 |
| | TCCACTTCACCTTGAAGAAGGTGACGGTGGCACTGTAGCACACGC | |
| GlnPr1523 | ATATACCGGTGGAGAGCTGTGCGGAGGCGCAGGACGGGGAGCTGGAC | 16 |
| | GGGCTGTG | |
| GlnPr1524 | ATATTTCGAATCACTAGGCCCCCTGTCCGATCATGTTCCTGTAGTCGGG | 17 |
| | GATGATGGTC | |
| GlnPr1649 | TCTCCACCGGTTGCAGCTCTGTTCCCTCCTCCCTCGGTTAG | 18 |
| GlnPr1650 | GCGCCATCGATACAGACATGATAAGATACATTG | 19 |
| GlnPr1651 | GCGCGATCGATACTTGTTTATTGCAGCTTATAATGG | 20 |
| GlnPr1689 | ATATGCTAGCCCACCATGCGGAGCCCTGCCCAGCTGCTGTTCCTGCTG | 21 |
| | CTGCTG | |
| GlnPr1690 | TGCTGTTCCTGCTGCTGCTGTGGATCCCTGGCACCAACGCCGAGGTGC | 22 |
| | AGCTGGTCGAGTC | |
| GlnPr1725 | ATATACTAGTCCACCATGCGGAGCCCTGCCCAGCTGCTGTTCCTGCTG | 23 |
| | CTGCTG | |
| GlnPr1726 | CTGTTCCTGCTGCTGCTGTGGATCCCTGGCACCAACGCCCAGGTGCAG | 24 |
| | CTGCAGCAGTC | |
| GlnPr1727 | CTGTTCCTGCTGCTGCTGTGGATCCCTGGCACCAACGCCATGGAGACA | 25 |
| | GACACACTCC | |
| GlnPr1735 | GACTTCGAATCATCAATGATGGTGGTGGTGATGGGCGCTGGCGAGGA | 26 |
| | GCAGGTCGAACAGGAGCAGCTTG | |
| GlnPr1760 | CCGCATCGATTTATCATTTACCCGGGCTCAGGCTCAG | 27 |
| GlnPr1799 | ATATACCGGTGGAGGCCCTGTGGCTGGGCGTGCTGAGACTGCTGCTGT | 28 |
| | TCAAGCTGCTCCTGTTCGACCTG | |
| GlnPr1800 | GTAAGCTTTCATCATTTACCCGGAGACAGGGAGAGGCTCTTCTGCGTG | 29 |
| | AATCGGTTGTGC | |
| GlnPr1801 | GCGCGAAGCTTTCATCATTTACCCGGAGACAGGGAGAGGCTCTTCTGC | 30 |
| | GTATAATGATTG | |
| GlnPr1840 | ATATACCGGTGGAGGCCCTGTGGCTGGGCGTGCTG | 31 |
| GlnPr2099 | TCCTCGGGGGGAGATCCACCACCACCTGAGCCAGGGAGGAGGGAAG | 32 |
| GlnPr2100 | GCGCTTCGAATCATCACAGAAACACGGTCTG | 33 |
| GlnPr2101 | TGGTGGTGGATCTCCCCCCGAGGACCCCCCTGACTCCAAG | 34 |
| GlnPr2102 | TCCTCGGGGGGAGATCCACCACCACCTGTTCCCTCCTCCCTCGGTTAG | 35 |
| GlnPr2160 | CCAAGCTTTCATCATTTGCCCGGAGACAGGGAGAGG | 312 |
| GlnPr2161 | CCAAGCTTTCATCATTTACCGGGAGACAGGGAGAGGCTCTTC | 313 |
| GlnPr2162 | GTCCTCGGGGGGAGATCCACCACCACCTGTCCCAGAAGGAGACGGTT | 314 |
| | AG | |
| GlnPr2163 | GTCCTCGGGGGGAGATCCACCACCACCTGTGCAATCCTCCCAGGGTTA | 315 |
| | G | |
| GlnPr2164 | GTCCTCGGGGGGAGATCCACCACCACCTGTGCAATAAGGACAGGGTT | 316 |
| | AG | |
| GlnPr2165 | GTCCTCGGGGGGAGATCCACCACCACCTGTCCCCTCAGGTCTCGGTTA | 317 |
| | G | |
| GlnPr2166 | GTCCTCGGGGGGAGATCCACCACCACCTGAGCCAGGTAGCAGGGAAG | 318 |
| | GG | |
| GlnPr2294 | CTACCAAGCTTTCATCATTTACCCGGAGACAGGGAGAGGCTCTTCTGC | 261 |
| | GTGTAGTGGTTGTGCAAGGCCTCG | |
| GlnPr2376 | GGCCGTTCGAATCATCACTTGCAGAAGCACTTGATAATC | 219 |
| GlnPr2377 | GTCATTTCGAATCATCAATGATGGTGGTGGTGATGGGCGCTGGCGATA | 220 |
| | ATCACGACGATCACGAC | |
| GlnPr2378 | TTGAACAGCAGCAGTCTCAGCACGCCCAGCCACAGGGCCCCGTCCAG | 221 |
| | CTCCCCGTCCTG | |
| GlnPr2379 | CTGAGACTGCTGCTGTTCAAGCTGCTCCTGTTCGACCTGCTCCTCAAGT | 222 |
| | GGATCTTCTCCTCGGTG | |
| GlnPr2380 | GCTTGAACAGCAGCAGTCTCAGCACGCCCAGCCACAGGGCATTCTTGG | 223 |
| | AGTCAGGGGGGTCCTC | |
| GlnPr2381 | CAGGACCGGTGCTTGCAGAAGCACTTGAGGAGCAGGTCGAACAGGAG | 224 |
| | CAGCTTGAACAGCAGCAGTCTC | |
| GlnPr2382 | GCTTGAACAGCAGCAGTACCAGCACGCCCAGCCACAGGGCATTCTTG | 225 |
| | GAGTCAGGGGGGTCCTC | |
| GlnPr2383 | CAGGACCGGTGCTTGCAGAAGCACTTGAGGAGCAGGTCGAACAGGAG | 226 |
| | CAGCTTGAACAGCAGCAGTACC | |
| GlnPr2384 | GACCGGTGCTTGCAGAAGCACTTGAGGAGCAGGTCGAACAGGAGCAG | 227 |
| | CACGAACAGCAGCAGTCTC | |
| GlnPr2385 | GACCGGTGCTTGCAGAAGCACTTGAGGAGCAGGACGAACAGGAGCAG | 228 |
| | CTTGAACAGCAGCAGTCTC | |
| GlnPr2386 | CAGGACCGGTGCTTGCAGAAGCACTTGAGGAGCAGGTCGAACAGGAG | 229 |
| | CAGCACGAACAGCAGCAGTACC | |
| GlnPr2387 | CAGGACCGGTGCTTGCAGAAGCACTTGAGGAGCAGGACGAACAGGAG | 230 |
| | CAGCTTGAACAGCAGCAGTACC | |
| GlnPr2388 | GACCGGTGCTTGCAGAAGCACTTGAGGAGCAGGACGAACAGGAGCAG | 231 |
| | CACGAACAGCAGCAGTCTC | |
| GlnPr2389 | CAGGACCGGTGCTTGCAGAAGCACTTGAGGAGCAGGACGAACAGGAG | 232 |
| | CAGCACGAACAGCAGCAGTACC | |
| GlnPr2390 | GGTCGTGATCGTCGTGATTATCAAGTGGATCTTCTCCTCGGTGGTGG | 233 |
| GlnPr2391 | CCACCACCGAGGAGAAGATCCACTTGATAATCACGACGATCACGACC | 234 |
| GlnPr2392 | CTTCGGCGCCACCGTGGTCGTGATCGTCGTGATTATCAAGTG | 235 |
| GlnPr2393 | CAGGACCGGTGCTTGCAGAAGCACTTGATAATCACGACGATC | 236 |
| GlnPr2394 | CTTCGGCGCCACCGTGATCGTCGTGATTATCAAGTGCTTC | 237 |
| GlnPr2395 | CAGGACCGGTGCTTGCAGAAGCACTTGATAATCACGAC | 238 |
| GlnPr2396 | CTTCGGCGCCGTGGTCATTGTAACCGTGGTCGTGATCGTCGTG | 239 |
| GlnPr2397 | CAGGACCGGTGCTTGCAGAAGCACTTGATAATCACGACGATCACGAC | 240 |
| | CAC | |
| GlnPr2398 | CTTCGGCGCCGTGGTCATTGTAGTCGTAACCGTGGTCGTGATCGTCGT | 241 |
| | G | |
| GlnPr2399 | CAGGACCGGTGCTTGCAGAAGCACTTGATAATCACGACGATCACGAC | 242 |
| | CACGG | |
| GlnPr2400 | GAGCCACCAGTGCCAGCAGAGCCAGCACAGGTCCCAGGATCAGAGCC | 243 |
| | AGATTCTTGGAGTCAGGGGGGTC | |
| GlnPr2401 | CAGGACCGGTGCTTGCAGAAGCACTTCCACAGTCCCAGCACTCCCAGA | 244 |
| | GCCACCAGTGCCAGCAG | |
| GlnPr2402 | CGATGCCCAGCAGCAGGAGCAACACCAGGATCACGATGATGGCGGCG | 245 |
| | ATATTCTTGGAGTCAGGGGGGTC | |
| GlnPr2403 | CAGGACCGGTGCTTGCAGAAGCACTTCCAGAACCACCACATCAGGCC | 246 |
| | GATGCCCAGCAGCAGGAG | |
| GlnPr2404 | GATAAACAGCAGCAGGCCAGCCACTCCGCCCAGCACAATCAGGGCCA | 247 |
| | TATTCTTGGAGTCAGGGGGGTC | |
| GlnPr2405 | CAGGACCGGTGCTTGCAGAAGCACTTGAAGAAGATGCCCAGGCCGAT | 248 |
| | AAACAGCAGCAGGCC | |
| GlnPr2406 | GTCCAATCAGCACGATGCCAGCCACCACGCCGGCCACGATAGGGATG | 249 |
| | ATATTCTTGGAGTCAGGGGGGTC | |
| GlnPr2407 | CAGGACCGGTGCTTGCAGAAGCACTTCCAGATCAGCAGCAGGGCCAG | 250 |
| | TCCAATCAGCACGATGCC | |
| GlnPr2408 | CCAGGAAGATGATCACGAACAGCAGGATGCCAGCGATCACGCCAGCG | 251 |
| | ATATTCTTGGAGTCAGGGGGGTC | |
| GlnPr2409 | CAGGACCGGTGCTTGCAGAAGCACTTCATCACGAGCACCACGCCCAG | 252 |
| | GAAGATGATCACGAA | |
| GlnPr2410 | GCAGGATGGCGAACAGGATGCCGAAGATGATGGGGATCACGACCAGA | 253 |
| | GCATTCTTGGAGTCAGGGGGGTC | |
| GlnPr2411 | CAGGACCGGTGCTTGCAGAAGCACTTGATGAACACCAGCACCAGCAG | 254 |
| | GATGGCGAACAGGATG | |
| GlnPr2412 | CTTCGGCGCCGTGATTACCGTGGTCGTGATCGTCGTGATTATCAGGTC | 255 |
| | CCAGCAGGAGGCCGCCG | |
| GlnPr2413 | GTCATTTCGAATCATCAGAAGAACTTCTTGGCGGCGGCCTCCTGCTGG | 256 |
| | GAC | |
| GlnPr2414 | GTCATTTCGAATCATCACCACCACTTCTTGGCGGCGGCCTCCTGCTGG | 257 |
| | GAC | |
| GlnPr2415 | GGTCATTTCGAATCATCAGGAGCCCTTCAGGGTCAGGAACATGATAAT | 258 |
| | CACGACGATCACGAC | |

### Restriction Digest

For all restriction digests around 1 g of plasmid DNA (quantified with NanoDrop, ND-1000 Spectrophotometer (Thermo Scientific, Wilmington, Del., USA)) was mixed to 10-20 units of each enzyme, 41 of corresponding 10×NEBuffer (NEB, Ipswich, Mass., USA), and the volume was made up to 40 l with sterile H₂O. The reactions were incubated for 1 h at the temperature necessary for the enzymes.

After each preparative digestion of backbone, 1 unit of Calf Intestinal Alkaline Phosphatase (CIP; NEB, Ipswich, Mass., USA) was added and the mix was incubated 30 min at 37° C.

### PCR Clean Up

To allow digestion, all PCR fragments were cleaned prior to restriction digests using the Macherey Nagel Extract II kit (Macherey Nagel, Oensingen, Switzerland) or Gel and PCR clean-up kit (new brand name of the same kit) following the manual of the manufacturer using 40 μl of elution buffer. This protocol was also used for changing buffers of DNA samples.

### DNA Extraction

Gel electrophoresis was performed using 1-2% agarose gels. These were prepared using the necessary amount of UltraPure™ Agarose (Invitrogen, Carlsbad, Calif., USA) and 1× Tris Acetic Acid EDTA buffer (TAE, pH 8.3; Bio RAD, Munich, Germany). For staining of DNA 1 l of Gel Red Dye (Biotum, Hayward, Calif., USA) was added to 100 ml of agarose gel. As size marker 21 of the 1 kb DNA ladder (NEB, Ipswich, Mass., USA) was used. The electrophoresis was run for around 1 hour at 125 Volts.

The bands of interests were cut out from the agarose gel and purified using the kit Extract II (Macherey-Nagel, Oensingen, Switzerland) or Gel and PCR clean-up kit (new brand name of the same kit) or Qiaquick Gel extraction kit (Qiagen, Hilden, Germany), following the manual of the manufacturer using 40 μl of elution buffer.

### Ligation

For each ligation, 41 of insert was mixed with 1 l of vector, 400 units of ligase (T4 DNA ligase, NEB, Ipswich, Mass., USA) and 1 l of 10× ligase buffer (T4 DNA ligase buffer; NEB, Ipswich, Mass., USA), filled up to a 10 l final volume using UHP water. The mix was incubated for 1-2 h at RT.

Transformation of Ligation Products into Competent Bacteria

For transformation of ligation products, the competent bacteria “TOP 10” (One Shot® TOP 10 Competent E. coli; Life Technologies, Carlsbad, Calif., USA) were used. 25-50 l of bacteria were thawed on ice for 5 minutes. Then, 3-5 l of ligation product were added to competent bacteria and incubated for 20-30 min on ice before a short heat shock of 1 min at 42° C. Then, 500 l of S.O.C medium (Invitrogen, Carlsbad, Calif., USA) were added per tube and incubate d for 1 h at 37° C. under agitation. Finally, the bacteria were put on a LB plate with ampicillin or kanamycin (Sigma-Aldrich, St. Louis, Mo., USA) and incubated overnight at 37° C.

### Minipreparation of Plasmids

For minipreparation, colonies of transformed bacteria were grown for 6-16 hours in 2.5 ml of LB and ampicillin or kanamycin at 37° C., 200 rpm. The DNA was extracted with a plasmid purification kit for E. coli (NucleoSpin QuickPure or NucleoSpin Plasmid (Macherey Nagel, Oensingen, Switzerland) or Qiagen QuickLyse Miniprep (Qiagen)), following the manufacturer's manual.

Plasmid DNA from minipreparations was quantified once with the NanoDrop ND-1000 Spectrophotometer (Thermo Scientific, Wilmington, Del., USA) by measuring the absorbance at 260 nm and assessing the ratio of the OD260 nm/OD280 nm that had to be between 1.8 and 2. A control digestion was performed before sending the sample to Fasteris SA (Plan-les-Ouates, Switzerland) for sequence confirmation.

### Midipreparation of Plasmids

For midipreparation, transformed bacteria were grown at 37° C. overnight in 200 ml of LB and ampicillin (or kanamycin). Then, the culture was centrifuged 20 min at 725 g and the plasmid was purified using a commercial kit (NucleoBond Xtra Midi; Macherey Nagel, Oensingen, Switzerland) following the protocol provided in the manufacturer's manual.

Plasmid DNA from midipreparation was quantified three times with the NanoDrop ND-1000 Spectrophotometer, by measuring the absorbance at 260 nm and assessing the ratio of the OD260 nm/OD280 nm that had to be between 1.8 and 2 as confirmed by restriction digest. The samples were then sent for sequencing (Fasteris SA, Plan-les-Ouates, Switzerland).

### Maxipreparation of Plasmids

For maxipreparation, transformed bacteria were grown at 37° C. overnight in 500 ml of LB and ampicillin (or kanamycin). The culture was centrifuged for 20 min at 725 g and the plasmid was purified using a commercial kit (NucleoBond PC500 or NucleoBond Xtra Maxi; Macherey Nagel, Oensingen, Switzerland) following the protocol provided in the manufacturer's manual.

Plasmid DNA from maxipreparation was quantified three times with the NanoDrop ND-1000 Spectrophotometer, by measuring the absorbance at 260 nm and assessing the ratio of the OD260 nm/OD280 nm that had to be between 1.8 and 2 as confirmed by restriction digest. The samples were then sent for sequencing (Fasteris SA, Plan-les-Ouates, Switzerland).

### Gigapreparation of Plasmids

For gigapreparation, transformed bacteria were grown at 37° C. overnight in 1500 ml of LB and ampicillin (or kanamycin). The culture was centrifuged for 20 min at 725 g and the plasmid was purified using a commercial kit (NucleoBond PC10000; Macherey Nagel, Oensingen, Switzerland) following the protocol provided in the manufacturer's manual.

Plasmid DNA from gigapreparation was quantified three times with the NanoDrop ND-1000 Spectrophotometer, by measuring the absorbance at 260 nm and assessing the ratio of the OD260 nm/OD280 nm that had to be between 1.8 and 2 as confirmed by restriction digest. The samples were then sent for sequencing (Fasteris SA, Plan-les-Ouates, Switzerland).

### Results

### Cloning of the Heavy Chain of an IgG1 Antibody

The gene coding for the IgG1 heavy chain used in this patent was optimized for expression in Chinese hamster cells by GeneArt (Regensburg, Germany). The variable part and the constant part of the heavy chain were provided in two different plasmids by GeneArt. After reception, GeneArt constructs were solubilised in water and the heavy chain constant region and the heavy chain variable region were cloned together using the enzymes KpnI and ApaI. This work was performed by an external CRO and the fused product was delivered to Glenmark. After sequencing of this fused construct, a sequence variation in the heavy chain constant region compared to the theoretical sequence was identified in the CH2 domain of the Fc region (EEMTK to DELTK). Primers were designed to change this region by megaprimer PCR and simultaneously introduced convenient restriction sites 5′ and 3′ of the open reading frame.

A first PCR using the primers GlnPr497 (SEQ ID NO:2) and GlnPr498 (SEQ ID NO:3) was performed on the original construct. The obtained amplicon (304 bps) was used in different concentrations as a megaprimer in a second PCR, using the second primer GlnPr501 and the original construct as template. The final PCR product was gel-purified and cloned into the shuttle vector pCR-blunt (Zero Blunt® PCR Cloning Kit, Invitrogen, LifeTechnologies). After miniprep screening it appeared that although the insert sequence was correct, the restriction sites 5′ and 3′ to the open reading frame were missing. Therefore the insert was re-amplified using the primers GlnPr501 (SEQ ID NO:4) and GlnPr498 (SEQ ID NO: 3) and a higher annealing temperature (64° C.). The amplicon was gel-purified and cloned into pCR-blunt (Zero Blunt® PCR Cloning Kit, Invitrogen, LifeTechnologies). One miniprep with the correct restriction pattern was chosen for subcloning in pSEL1, a shuttle vector, using restriction enzymes HindIII and XbaI. After miniprep screening, a maxiprep was produced. The insert of this maxiprep was again cut out using the restriction enzymes XbaI and HindIII and cloned into the backbone of the plasmid pGLEX41 (GSC281, SEQ ID NO: 304), opened with the same enzymes and treated with CIP. pGLEX41 is the standard in-house vector for expression and contains an expression cassette consisting of the mouse CMV promoter followed by a constitutively spliced intron, a multiple cloning site (MCS) and a SV40 poly(A) sequence. After miniprep screening, a maxiprep of pGLEX41_HC was produced and received the plasmid batch number GSC314. Further midipreps or gigapreps of the same DNA received the plasmid batch numbers GSC314a, GSC314b, GSC314c, GSC314d and GSC314e (all these preps were confirmed to be identical by DNA sequencing and will be referred to as GSC314 plasmid (SEQ ID NO: 48)).

### Cloning of the Alternate Splicing Expression Vector for Membrane Displayed Heavy Chain of an IgG1 Antibody

The coding region of the heavy chain of an antibody of the IgG1 format was amplified by PCR using the primers GlnPr1518 (SEQ ID NO: 11), GlnPr1519 (SEQ ID NO: 12) and GlnPr1520 (SEQ ID NO: 13) and the plasmid pGLEX41_HC (GSC314, SEQ ID NO: 48) as template. The resulting PCR product contained a NheI restriction site at the 5′end, and the 3′ end of the amplicon was modified in order to contain precisely the last 40 bps of NCBI database entry for the ighg1 gene (SEQ ID NO: 65) as well as a HindIII restriction site. The 3′end modification was done in order to reconstitute the natural splice donor site in the C-terminal region of the constant part of the IgG1 heavy chain. The PCR product was cut using the restriction enzymes NheI and HindIII and cloned in pGLEX41 (GSC281, SEQ ID NO: 304), cut using the same enzymes and CIPed. The resulting vector was called pGLEX41_HC-AS (plasmid batch number GSC3836, SEQ ID NO: 287) and was confirmed by restriction digest and sequence analysis.

The chicken troponin (cTNT) intron number 4 (SEQ ID NO: 69) was amplified from an in-house plasmid containing its sequence (GSC2819, SEQ ID NO: 67) with the primers GlnPr1516 (SEQ ID NO: 9) and GlnPr1517 (SEQ ID NO: 10). Troponin is expressed exclusively in cardiac muscle and embryonic skeletal muscle. Over 90% of the mRNAs include the exon in early embryonic heart and skeletal muscle, whereas >95% of mRNAs in the adult exclude the exon (Cooper & Ordahl (1985) J Biol Chem, 260(20): 11140-8). The primers added a HindIII site 5′ and an AgeI site followed by BstBI to the 3′ terminus. The amplicon was gel-purified, digested using HindIII and BstBI and cloned into the vector pGLEX41_HC-IgAS (GSC3836, SEQ ID NO: 287) opened using the same enzymes and CIPed. The resulting vector was called pGLEX41_HC-cTNTintron4 (plasmid batch number GSC3848, SEQ ID NO: 289) and was confirmed by restriction digest and sequence analysis.

The sequence coding for the transmembrane region of the human ighg1 gene (M1M2) was assembled using plasmids GlnPr1521 (SEQ ID NO: 14) and GlnPr1522 (SEQ ID NO: 15). These primers overlap partially and were completed to double stranded DNA using PCR. The blunt-ended PCR product was cloned into pCR-blunt (Zero Blunt® PCR Cloning Kit, Invitrogen, LifeTechnologies), leading to the plasmid pCRblunt-M1M2 which was confirmed by sequencing. The insert was re-amplified with the primers GlnPr1523 (SEQ ID NO: 16) and GlnPr1524 (SEQ ID NO: 17) to complete the M1M2 transmembrane domain sequence. The amplicon was gel-purified, digested using AgeI and BstBI, and cloned in vector pGLEX41_HC-cTNTintron4 (GSC3848, SEQ ID NO: 289). The resulting plasmid was named pGLEX41_HC-I4-M1M2-M1M2-M1M2 and was produced in maxiprep scale, received the plasmid batch number GSC3899 and was confirmed by sequence analysis (SEQ ID NO: 36).

In order to increase the splice ratio between expressed and membrane displayed antibody, the number of pyrimidines in the poly(Y) tract upstream of the exon coding for the transmembrane region was reduced. For this the I4(0Y) fragment (SEQ ID NO: 70) was amplified from an in-house vector (GSC3469, SEQ ID No. 68) using the primers GlnPr1516 (SEQ ID NO: 9) and GlnPr1649 (SEQ ID NO: 18). The amplicon was digested using the restriction enzymes HindIII and AgeI and cloned into the backbone of the pGLEX41_HC-I4-M1M2-M1M2-M1M2 (GSC3899, SEQ ID NO: 36) backbone opened using the same enzymes and treated with CIP. After miniprep screening, the midiprep pGLEX41_HC-I4(0Y)-M1M2-M1M2-M1M2 was prepared, received the batch number GSC4398 and was confirmed by sequencing (SEQ ID NO: 37).

A poly(A) was introduced in the intron separating the exon coding for the IgG1 and the alternate exon coding for the transmembrane region. The SV40 poly(A) signal was amplified from an in-house vector (GSC3469, SEQ ID NO: 305) with the primers GlnPr1650 (SEQ ID NO: 19) and GlnPr1651 (SEQ ID NO: 20). The amplicon was digested using the restriction enzyme ClaI and cloned into the vector pGLEX41_HC-M1M2-M1M2-M1M2 (GSC3899, SEQ ID NO: 36) digested using ClaI and CIPed, resulting in the I4(polyA) intron (SEQ ID NO: 71) After miniprep screening and confirmation of the right orientation of the insert, the pGLEX41_HC-I4(polyA)-M1M2-M1M2-M1M2 midiprep was prepared, received the batch number GSC4401 and was confirmed by sequencing (SEQ ID NO: 38).

The gastrin terminator sequence had been previously added to the SV40 poly(A) signal of an in-house vector (GSC23, SEQ ID NO: 306) sharing the same general backbone than pGLEX41 (GSC281, SEQ ID NO: 304) but bearing a different promoter. The fusion of SV40 poly(A) and gastrin terminator was designated as “SV40ter” (SEQ ID NO: 306) in the plasmid nomenclature. The expression cassette of GSC23 (SEQ ID NO: 306) (promoter+coding sequence) was replaced by the expression cassette of pGLEX41_HC-I4-M1M2-M1M2-M1M1 (GSC3899, SEQ ID NO: 36) except for the SV40 poly(A) signal (i.e. promoter and coding sequence). The insert was cut out with the restriction enzymes BstBI and NruI and cloned into the backbone of GSC23 (SEQ ID NO: 306) digested with the same enzymes and treated with CIP. After miniprep screening, a midiprep of plasmid pGLEX41_HC-I4-M1M2-M1M2-M1M2-SV40ter was prepared, received the plasmid batch number GSC4402 and was confirmed by sequencing (SEQ ID NO: 39).

Another preferred construct comprises a poly(A) signal with the gastrin terminator in the intron. The sequence of I4(SV40ter) is given in SEQ ID NO: 77.

Cloning of the Alternate Splicing Expression Vector for Membrane Displayed Heavy Chain of an IgG1 Antibody Using the Human T-Cell Receptor Alpha Transmembrane Domain (PTCRA)

In order to replace the M1M2 transmembrane region in the alternate splicing constructs with the transmembrane domain of the human T-cell receptor alpha (PTCRA) gene, the complementary primers GlnPr1799 (SEQ ID NO: 28) and GlnPr1735 (SEQ ID NO: 26) were used for generation of the insert fragment. These primers overlap partially and were completed to double stranded DNA using PCR. The insert fragment was cut using AgeI and BstBI and cloned into the backbone of pGLEX41_HC-I4-M1M2-M1M2-M1M2 (GSC3899, SEQ ID NO: 36) digested with the same enzymes and treated with CIP. As a part of the insert was missing from the resulting miniprep a new amplification was performed on this miniprep using the primers GlnPr1799 (SEQ ID NO: 28) and GlnPr269 (SEQ ID NO: 1). The amplified insert fragment was digested with AgeI and BstBI and cloned into the backbone of pGLEX41_HC-I4(0Y)-M1M2-M1M2-M1M2 (GSC4398, SEQ ID NO: 37) digested with the same enzymes and treated with CIP. One miniprep was confirmed by sequencing, and was used for another PCR with the primers GlnPr1840 (SEQ ID NO: 31) and GlnPr269 (SEQ ID NO: 1). The amplicon was digested with AgeI and BstBI and cloned in the backbones pGLEX41_HC-I4-M1M2-M1M2-M1M2 (GSC3899, SEQ ID NO: 36), pGLEX41_HC-I4(0Y)-M1M2-M1M2-M1M2 (GSC4398, SEQ ID NO: 37), pGLEX41_HC-I4(polyA)-M1M2-M1M2-M1M2 (GSC4401, SEQ ID NO: 38) and pGLEX41_HC-I4-M1M2-M1M2-M1M2-SV40ter (GSC4402, SEQ ID NO: 39) digested using the same enzymes and treated with CIP. The ligations resulted in the midipreps pGLEX41_HC-I4-PTCRA (GSC5854, SEQ ID NO: 40), pGLEX41_HC-I4(0Y)-PTCRA (GSC5855, SEQ ID NO: 41), pGLEX41_HC-I4(polyA)-PTCRA (GSC5857, SEQ ID NO: 42) and pGLEX41_HC-I4-PTCRA-SV40ter (GSC5856, SEQ ID NO: 43), respectively. All the midipreps were verified by sequencing.

Cloning of the Alternate Splicing Expression Vector for Membrane Displayed Heavy Chain of an IgG1 Antibody Using an M1M2-PTCRA Fusion Construct

The insert coding for the M1M2-PTCRA fusion construct was ordered from GeneArt with the name GeneArt_Seq32 (SEQ ID NO: 64). This fragment contains the regular M1M2 transmembrane region of an IgG1 heavy chain, except for the transmembrane domain that was replaced by the transmembrane domain of the T-cell receptor alpha subunit (PTCRA). The plasmid provided by GeneArt was cut using the restriction enzymes AgeI and BstBI and the purified insert was ligated in the backbone of pGLEX41_HC-I4-M1M2-M1M2-M1M2 (GSC3899, SEQ ID NO: 36), pGLEX41_HC-I4(0Y)-M1M2-M1M2-M1M2 (GSC4398, SEQ ID NO: 37), pGLEX41_HC-I4(polyA)-M1M2-M1M2-M1M2 (GSC4401, SEQ ID NO: 38) and pGLEX41_HC-I4-M1M2-M1M2-M1M2-SV40ter (GSC4402, SEQ ID NO: 39), respectively, digested with the same enzymes and treated with CIP.

The ligations resulted in the midipreps pGLEX41_HC-I4-M1M2-PTCRA-M1M2 (GSC6030, SEQ ID NO: 44), pGLEX41_HC-I4(0Y)-M1M2-PTCRA-M1M2 (GSC6048, SEQ ID NO: 45), pGLEX41_HC-I4(polyA)-M1M2-PTCRA-M1M2(GSC6047, SEQ ID NO: 46) and pGLEX41_HC-I4-M1M2-PTCRA-M1M2-SV40ter (GSC6046, SEQ ID NO: 47). All midipreps were confirmed by sequencing.

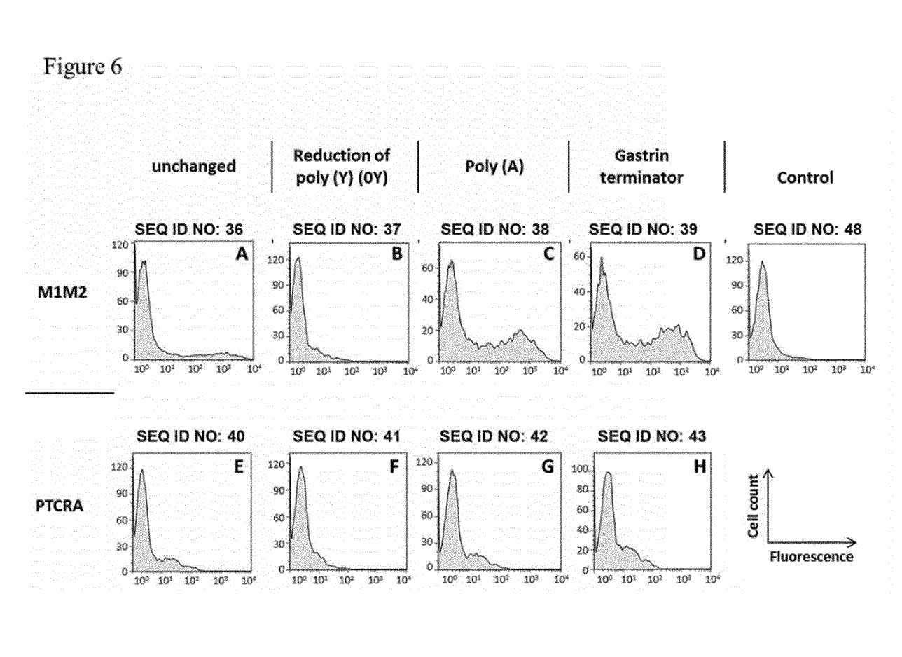

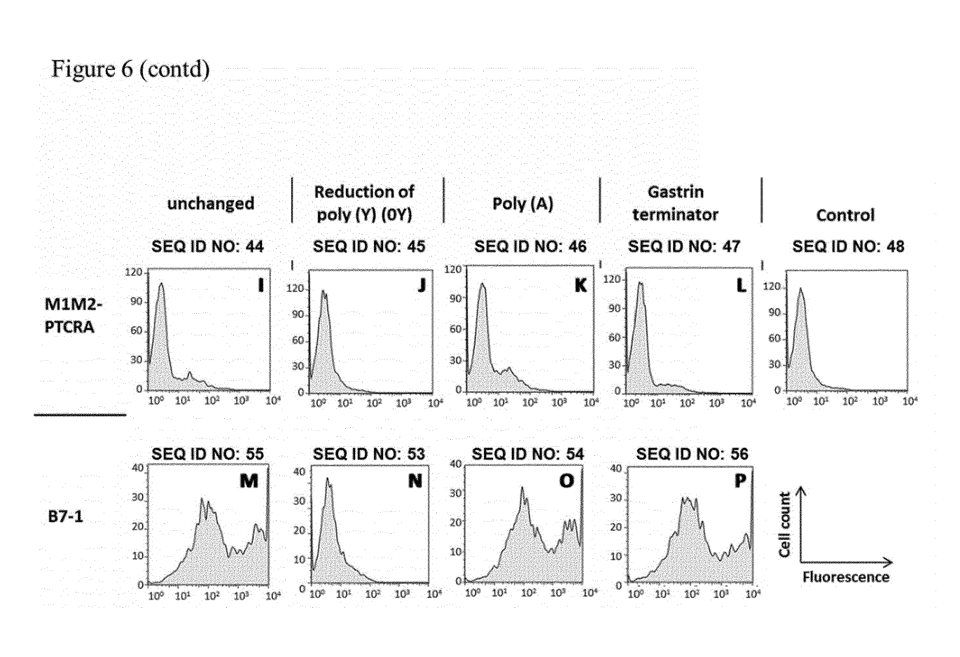

A first M1M2-PTCRA fusion was generated, these constructs allowed membrane display of the IgG1 and showed the same behaviour regarding modifications of introns as the native M1M2 constructs (see FIG. 6 in Example 3).

A further construct pGLEX41_HC-I4-M1M2-PTCRA-M1M2_corrected was designed to allow a correct analysis of the impact of the modification of the transmembrane domain in the transmembrane region.

For this construct, a fusion PCR was performed. A portion of the cTNT 4 intron and the M1M2 extracellular part of the M1M2 transmembrane region were amplified from the plasmid pGLEX41_HC-I4-M1M2-M1M2-M1M2-(GSC3899, SEQ ID NO: 36) with the primers GlnPr1285 (SEQ ID NO: 259) and GlnPr2378 (SEQ ID NO: 221). The cytosolic tail of the M1M2 transmembrane region as well as the SV40 poly(A) signal were amplified with the primers GlnPr2379 (SEQ ID NO: 222) and GlnPr1650 (SEQ ID NO: 19) from the same template. The primers GlnPr2378 (SEQ ID NO: 221) and GlnPr2379 (SEQ ID NO: 222) allowed the creation of overlapping ends of the two PCR products creating the corrected PTCRA transmembrane domain. An overlapping PCR was then performed from these two first PCR products with the primers GlnP1285 (SEQ ID NO: 259) and GlnPr1650 (SEQ ID NO:19). The resulting fusion PCR product was digested with AgeI and BstBI and ligated in the backbone pGLEX41_HC-I4-M1M2-M1M2-M1M2 (GSC3899, SEQ ID NO: 36) digested with the same enzymes and treated with CIP. After miniprep screening, the midiprep pGLEX41_HC-I4-M1M2-PTCRA-M1M2_corrected was produced, received the plasmid batch number GSC11206 and was confirmed by sequencing (SEQ ID NO: 284).

Cloning of the Alternate Splicing Expression Vector for Membrane Displayed Heavy Chain of an IgG1 Antibody Using the Murine B7-1 Transmembrane Region

In order to change the transmembrane region to the transmembrane region of the murine B7-1 gene, fusion PCRs were performed.

First, the sequence coding for the murine B7-1 transmembrane region was amplified by PCR from an in-house construct (ordered from GeneArt as GeneArt_Seq43, SEQ ID NO: 66) using the primers GlnPr2100 (SEQ ID NO: 33) and GlnPr2101 (SEQ ID NO:34).

For cloning of vector pGLEX41_HC-I4(0Y)-B7-B7-B7, the fragment coding for cTNT-I4(0Y) was amplified by PCR from template pGLEX41_HC-I4(0Y)-M1M2-M1M2-M1M2 (GSC4398, SEQ ID NO: 37) using primers GlnPr1516 (SEQ ID NO: 9) and GlnPr2102 (SEQ ID NO:35). The sequence coding for the murine B7-1 transmembrane region was amplified by PCR from an in-house construct (ordered from GeneArt as GeneArt_Seq43, SEQ ID NO: 66) using the primers GlnPr2100 (SEQ ID NO: 33) and GlnPr2101 (SEQ ID NO:34). Both PCR products were fused using the primers GlnPr1516 (SEQ ID NO: 9) and GlnPr2100 (SEQ ID NO:33). The resulting PCR product was digested with BstBI and HindIII, and was ligated into pGLEX41_HC-I4-M1M2-M1M2-M1M2 (GSC3899, SEQ ID NO: 36) digested with the same enzymes and treated with CIP. After miniprep screening, the midiprep of pGLEX41_HC-I4(0Y)-B7-B7-B7 was produced, received the batch number GSC7057 and was confirmed by restriction digest and sequencing (SEQ ID NO: 53).

For cloning of vector pGLEX41_HC-I4(polyA)-B7-B7-B7, the fragment coding for cTNT-I4(polyA) was amplified by PCR from template pGLEX41_HC-I4(polyA)-M1M2-M1M2-M1M2 (GSC4401, SEQ ID NO: 38) using primers GlnPr1516 (SEQ ID NO: 9) and GlnPr2099 (SEQ ID NO:32). The sequence coding for the murine B7-1 transmembrane region was amplified by PCR from an in-house construct (ordered from GeneArt as GeneArt_Seq43, SEQ ID NO: 66) using the primers GlnPr2100 (SEQ ID NO: 33) and GlnPr2101 (SEQ ID NO:34). Both PCR products were fused using the primers GlnPr1516 (SEQ ID NO: 9) and GlnPr2100 (SEQ ID NO:33). The resulting PCR product was digested with BstBI and HindIII, and was ligated into pGLEX41_HC-I4-M1M2-M1M2-M1M2 (GSC3899, SEQ ID NO: 36) digested with the same enzymes and treated with CIP. After miniprep screening, the resulting pGLEX41_HC-I4(polyA)-B7-B7-B7 midiprep was given the batch number GSC7058 and was confirmed by restriction digest and sequencing (SEQ ID NO: 54).

For cloning of the vector pGLEX41_HC-I4-B7-B7-B7, the cTNT-I4 was amplified by PCR from template pGLEX41_HC-I4-M1M2-M1M2-M1M2 (GSC3899, SEQ ID NO: 36) using primers GlnPr1516 (SEQ ID NO: 9) and GlnPr2099 (SEQ ID NO:32). The sequence coding for the murine B7-1 transmembrane region was amplified by PCR from an in-house construct (ordered from GeneArt as GeneArt_Seq43, SEQ ID NO: 66) using the primers GlnPr2100 (SEQ ID NO: 33) and GlnPr2101 (SEQ ID NO:34). Both PCR products were fused using the primers GlnPr1516 (SEQ ID NO: 9) and GlnPr2100 (SEQ ID NO:33). The resulting PCR product was digested with BstBI and HindIII, and was ligated into pGLEX41_HC-I4-M1M2-M1M2-M1M2 (GSC3899, SEQ ID NO: 36) digested with the same enzymes and treated with CIP. After miniprep screening, the resulting pGLEX41_HC-I4-B7-B7-B7 midiprep was given the batch number GSC7056 and was confirmed by restriction digest and sequencing (SEQ ID NO: 55).

For cloning of vector pGLEX41_HC-I4-B7-B7-B7-SV40ter, the same PCR product as for pGLEX41_HC-I4-B7-B7-B7 construct was used, digested with HindIII and BstBI and ligated into pGLEX41_HC-I4-M1M2-M1M2-M1M2-SV40ter (GSC4402, SEQ ID NO: 39) digested with the same enzymes and treated with CIP. After miniprep screening, the resulting pGLEX41_HC-I4-B7-B7-B7-SV40ter midiprep was given the batch number GSC7059 and was confirmed by restriction digest and by sequencing (SEQ ID NO: 56).

Further Reduction of the Splice Ratio of Secreted to Membrane Displayed Heavy Chain of an IgG1 Antibody Using the Murine B7-1 Transmembrane Region

It has been shown that the membrane display of chimeric proteins is highly efficient using the murine B7-1 transmembrane region (Chou W-C et al., (1999) Biotechnol Bioeng, 65: 160-9; Liao K-W et al., (2001) Biotechnol Bioeng, 73: 313-23). It has also been shown herein that decreasing the amount of Ys in the poly(Y) tract of the splice-acceptor to 0 lowered the amount of displayed antibody on the cell membrane significantly (see FIG. 6F in Example 3).

In order to assess the effect of the reduction of the amount of pyrimidines in the poly(Y) tract of the intron, several constructs were produced by PCR fusion.

TABLE 3
Modification of the intron: Primer pairs and templates used for PCR.

| | Intron Sequence | Number of Y | | Primer pairs |
|---|---|---|---|---|
| Construct ID | SEQ ID NO | in the intron | Template PCR 1 | PCR 1 |
| pGLEX41_HC-I4(3Y)-I4-B7- | 72 | 3Y | GSC4340 | GlnPr1516 |
| B7-B7 | | | (SEQ ID NO: 307) | (SEQ ID NO: |
| | | | | 9)/GlnPr2165 |
| | | | | (SEQ ID |
| | | | | NO: 317) |
| pGLEX41_HC-I4(5Y)-I4-B7- | 73 | 10Y | GSC4222 | GlnPr1516 |
| B7-B7 | | | (SEQ ID NO: 308) | (SEQ ID NO: |
| | | | | 9)/GlnPr2164 |
| | | | | (SEQ ID |
| | | | | NO: 316) |
| pGLEX41_HC-I4(5Y-5)-I4- | 74 | 5Y | GSC2617 | GlnPr1516 |
| B7-B7-B7 | | | (SEQ ID NO: 309) | (SEQ ID NO: |
| | | | | 9)/GlnPr2163 |
| | | | | (SEQ ID |
| | | | | NO: 315) |
| pGLEX41_HC-I4(9Y)-I4-B7- | 75 | 9Y | GSC4335 | GlnPr1516 |
| B7-B7 | | | (SEQ ID NO: 310) | (SEQ ID NO: |
| | | | | 9)/GlnPr2162 |
| | | | | (SEQ ID |
| | | | | NO: 314) |
| pGLEX41_HC-I4(15Y-3′)-I4- | 76 | 25Y | GSC2332 | GlnPr1516 |
| B7-B7-B7 | | | (SEQ ID NO: 311) | (SEQ ID NO: |
| | | | | 9)/GlnPr2166 |
| | | | | (SEQ ID |
| | | | | NO: 318) |

A 1^{st} PCR allowed amplifying the desired intron from in-house backbones containing the modified introns. The primers and templates used for each construct are listed in Table 3. A 2^{nd} PCR (common for all constructs) allowed the amplification of the B7-1 transmembrane domains from the construct pGLEX41_HC-I4-B7-B7-B7 (GSC7056, SEQ ID NO: 55) with the primers GlnPr2101 (SEQ ID NO: 34) and GlnPr2100 (SEQ ID NO:33). After purification of these PCR products, the introns and transmembrane domain were fused by overlapping extension PCR with the primers GlnPr1516 (SEQ ID NO: 9) and GlnPr2100 (SEQ ID NO:33). The purified PCR products were then digested with HindIII and BstBI and ligated in the backbone of pGLEX41_HC-I4-B7-B7-B7 (GSC7056, SEQ ID NO: 55) digested with the same enzymes and treated with CIP. After miniprep screening, midipreps were produced for each of the constructs and verified by sequencing. The plasmid batch numbers attributed to each constructs are the following:

| | |
|---|---|
| pGLEX41_HC-I4(3Y)-I4-B7-B7-B7 | GSC9770 (SEQ ID NO: 62) |
| pGLEX41_HC-I4(5Y)-I4-B7-B7-B7 | GSC9763 (SEQ ID NO: 61) |
| pGLEX41_HC-I4(5Y-5)-I4-B7-B7-B7 | GSC9768 (SEQ ID NO: 60) |
| pGLEX41_HC-I4(9Y)-I4-B7-B7-B7 | GSC9949 (SEQ ID NO: 59) |
| pGLEX41_HC-I4(15Y-3′)-I4-B7-B7-B7 | GSC9950 (SEQ ID NO: 63) |

Another way to reduce the splicing was to weaken the splice donor site. The splice donor is characterized by a consensus sequence defining the 5′end of the intron. Therefore, two constructs were designed, where the DNA sequence of the splice donor consensus was modified without impact on the protein sequence expressed by the gene.

PCR amplifying the IgG1 heavy chain with modified 3′ end sequence was performed on the template pGLEX41_HC-I4-B7-B7-B7 (GSC7056, SEQ ID NO: 55) with the primer GlnPr1518 (SEQ ID NO: 11) and GlnPr2161 (SEQ ID NO: 313) for the SD_CCC construct and GlnPr1518 (SEQ ID NO: 11) and GlnPr2160 (SEQ ID NO: 314) for the SD_GGC construct. The PCR were digested with NheI and HindIII and ligated in the backbone pGLEX41_HC-I4-B7-B7-B7 (GSC7056, SEQ ID NO: 55) digested with the same enzymes and treated with CIP. After miniprep screening, the midipreps pGLEX41_HC-SD_CCC-I4-B7-B7-B7 (plasmid batch number GSC9764, SEQ ID NO: 58) and pGLEX41_HC-SD_GGC-I4-B7-B7-B7 (plasmid batch number GSC9765, SEQ ID NO: 57) were produced and confirmed by sequencing. The intron sequence of the construct pGLEX41_HC-SD_GGC-I4-B7-B7-B7 is given in SEQ ID NO: 78.

Replacement of B7-1 Transmembrane Domain by Transmembrane Domains of Similar Length with Only Hydrophobic Residues

To assess the effect of the transmembrane domain on the membrane display, several constructs were designed. Transmembrane helices containing mainly hydrophobic residues are thought to be favourable for the stabilization of the surface display, and thus, as 1st approach, the B7-1 transmembrane domain of the B7-1 transmembrane region was replaced by transmembrane domains of other proteins containing only hydrophobic residues. Moreover, as the length of the transmembrane helice might have an impact on surface display, these first constructs were only composed of transmembrane domains with a length similar to the B7-1 transmembane domain.

The sequences and characteristics of these transmembrane domains are summarized in Table 4.

TABLE 4
Transmembrane domains with 22-23 amino acids (all hydrophobic):
characteristics and sequences.

| Trans- | | | | | | SEQ |
|---|---|---|---|---|---|---|
| membrane | | Hydro- | | | | ID |
| domian | Length | phobic | Polar | Charged | Sequence | NO |
| B7 | 22 | 20 | 2 | 0 | TLVLFGAGFGAVITVVVIVVII | 173 |
| ACLV1 | 23 | 23 | 0 | 0 | LALILGPVLALLALVALGVLGLW | 174 |
| ANTR2 | 23 | 23 | 0 | 0 | IAAIIVILVLLLLLGIGLMWWFW | 175 |
| CD4 | 22 | 22 | 0 | 0 | MALIVLGGVAGLLLFIGLGIFF | 176 |
| ITB1 | 23 | 23 | 0 | 0 | IAGVIAGILLFVIIFLGVVLVM | 179 |
| PTPRM | 22 | 22 | 0 | 0 | IAGVIAGILLFVIIFLGVVLVM | 177 |
| TNR5 | 22 | 22 | 0 | 0 | ALVVIPIIFGILFAILLVLVFI | 178 |

TABLE 5
Primer pairs used for the cloning of
hydrophobic transmembrane domains.

| Trans- | | |
|---|---|---|
| membrane | | |
| domain | Primer 1^{st} PCR | Primers 2^{nd} PCR |
| ACVL1 | GlnPr1516 (SEQ ID NO: 9)/ | GlnPr1516 (SEQ ID NO: 9)/ |
| | GlnPr2400 (SEQ ID NO: 243) | GlnPr2401 (SEQ ID NO: 244) |
| ANTR2 | GlnPr1516 (SEQ ID NO: 9)/ | GlnPr1516 (SEQ ID NO: 9)/ |
| | GlnPr2402 (SEQ ID NO: 245) | GlnPr2403 (SEQ ID NO: 246) |
| CD4 | GlnPr1516 (SEQ ID NO: 9)/ | GlnPr1516 (SEQ ID NO: 9)/ |
| | GlnPr2404 (SEQ ID NO: 247) | GlnPr2405 (SEQ ID NO: 248) |
| PTPRM | GlnPr1516 (SEQ ID NO: 9)/ | GlnPr1516 (SEQ ID NO: 9)/ |
| | GlnPr2408 (SEQ ID NO: 251) | GlnPr2409 (SEQ ID NO: 252) |
| TNR5 | GlnPr1516 (SEQ ID NO: 9)/ | GlnPr1516 (SEQ ID NO: 9)/ |
| | GlnPr2410 (SEQ ID NO: 253) | GlnPr2411 (SEQ ID NO: 254) |
| ITB1 | GlnPr1516 (SEQ ID NO: 9)/ | GlnPr1516 (SEQ ID NO: 9)/ |
| | GlnPr2406 (SEQ ID NO: 249 | GlnPr2407 (SEQ ID NO: 250) |

The different transmembrane domains were created by 2 step PCR amplification of a portion of the cTNT intron 4 with primers modifying the 3′ end of the transmembrane domain. The template was always the pGLEX41_HC-I4-B7-B7-B7 (GSC7056, SEQ ID NO: 55) construct. For each construct, a first round PCR was performed with the respective primers listed in Table 5. After clean-up of this 1^{st} round PCR products, a 2^{nd} round of PCR was performed with the primers listed in the Table 5. After clean-up, these PCR products were digested with AgeI and ClaI and ligated in the backbone pGLEX41_HC-I4-B7-B7-B7 (GSC7056, SEQ ID NO: 55) digested with the same enzymes and treated with CIP. After confirmation of the sequences at the miniprep level, midiprep of the different constructs were produced and the following GSC numbers were assigned to these plasmid batches:

| | |
|---|---|
| pGLEX41-GBR200HC-I4-B7-ACVL1-B7 | GSC11213 (SEQ ID NO: 81) |
| pGLEX41-GBR200HC-I4-B7-ANTR2-B7 | GSC11214 (SEQ ID NO: 82) |
| pGLEX41-GBR200HC-I4-B7-CD4-B7 | GSC11210 (SEQ ID NO: 83) |
| pGLEX41-GBR200HC-I4-B7-PTPRM-B7 | GSC11205 (SEQ ID NO: 84) |
| pGLEX41-GBR200HC-I4-B7-TNR5-B7 | GSC11217 (SEQ ID NO: 85) |
| pGLEX41-GBR200HC-I4-B7-ITB1-B7 | GSC11756 (SEQ ID NO: 86) |

All midipreps were confirmed by sequencing.

Replacement of B7-1 Transmembrane Domain by Transmembrane Domains of Similar Length Containing Charged and/or Polar Residues

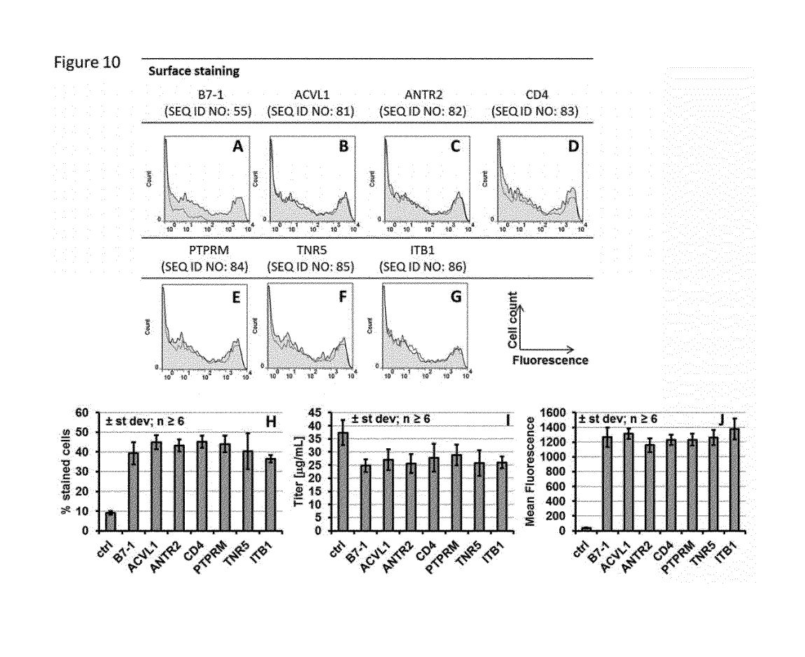

As transmembrane domains containing only hydrophobic residues have no impact on surface display (see FIG. 10 in Example 4), other transmembrane domains were chosen having a similar length than B7-1 transmembrane domain but containing one or more charged and/or polar residues.

The sequences and characteristics of these transmembrane domains are summarized in the Table 6.

TABLE 6
Transmembrane domains with 21-24 amino acids containing charged and/or
polar residues: characteristics and sequences.

| Trans- | | | | | | SEQ |
|---|---|---|---|---|---|---|
| membrane | | Hydro- | | | | ID |
| domian | Length | phobic | Polar | Charged | Sequence | NO |
| B7 | 22 | 20 | 2 | 0 | TLVLFGAGFGAVITVVVIVVII | 173 |
| IGF1R | 24 | 22 | 1 | 1 | LIIALPVAVLLIVGGLVIMLYVFH | 181 |
| 1B07 | 24 | 22 | 1 | 1 | GIVAGLAVLAVVVIGAVVAAVMCR | 180 |
| TRBM | 24 | 19 | 4 | 1 | GLLIGISIASLCLVVALLALLCHL | 182 |
| IL4RA | 24 | 16 | 8 | 0 | LLLGVSVSCIVILAVCLLCYVSIT | 183 |
| LRP6 | 23 | 15 | 8 | 0 | TNTVGSVIGVIVTIFVSGTVYFI | 184 |
| GpA | 23 | 19 | 4 | 0 | ITLIIFGVMAGVIGTILLISYGI | 185 |
| PTCRA | 21 | 18 | 0 | 3 | ALWLGVLRLLLFKLLLFDLLL | 186 |

TABLE 7
GeneArt constructs used for the cloning of transmembrane domains
with 21-24 amino acids containing charged and/or polar residues.

| Trans- | | |
|---|---|---|
| membrane | | |
| domain | GeneArt Seq name | GeneArt Seq SEQ ID NO |
| 1B07 | GeneArt_Seq84 | 266 |
| IGF1R | GeneArt_Seq 83 | 265 |
| TRBM | GeneArt_Seq 85 | 267 |
| IL4RA | GeneArt_Seq 86 | 268 |
| LRP6 | GeneArt_Seq 87 | 269 |
| GpA | GeneArt_Seq 94 | 276 |

The different transmembrane domains (except PTCRA) were ordered from GeneArt with the names indicated in the Table 7. Once received, GeneArt constructs were resuspended in water and then digested with AgeI and ClaI. The resulting inserts were ligated in the backbone pGLEX41_HC-I4-B7-B7-B7 (GSC7056, SEQ ID NO: 55) digested with the same enzymes and treated with CIP. After miniprep screening, midipreps were produced and received the following batch numbers:

| | |
|---|---|
| pGLEX41-GBR200HC-I4-B7-1B07-B7 | GSC11296 (SEQ ID NO: 87) |
| pGLEX41-GBR200HC-I4-B7-IGF1R-B7 | GSC11295 (SEQ ID NO: 88) |
| pGLEX41-GBR200HC-I4-B7-TRBM-B7 | GSC11390 (SEQ ID NO: 89) |
| pGLEX41-GBR200HC-I4-B7-IL4RA-B7 | GSC11391 (SEQ ID NO: 90) |
| pGLEX41-GBR200HC-I4-B7-LRP6-B7 | GSC11392 (SEQ ID NO: 91) |
| pGLEX41-GBR200HC-I4-B7-GpA-B7 | GSC11397 (SEQ ID NO: 92) |

For the construct pGLEX41_HC-I4-B7-PTCRA-B7, a 2 step PCR was performed. The cTNT intron with the B7 extracellular part of the B7-1 transmembrane region was amplified using the primers GlnPr1516 (SEQ ID NO: 9) and GlnPr2380 (SEQ ID NO: 223) and pGLEX41_HC-I4-B7-B7-B7 (GSC7056, SEQ ID NO: 55) as template. The resulting PCR product was re-amplified with the primers GlnPr1516 (SEQ ID NO: 9) and GlnPr2381 (SEQ ID NO: 224). The primers GlnPr2380 (SEQ ID NO: 223) and GlnPr2381 (SEQ ID NO: 224) allowed fusing the PTCRA transmembrane domain to the B7-1 extracellular part of the B7-1 transmembane region. The resulting PCR product was digested with ClaI and AgeI and recloned in the backbone pGLEX41_HC-I4-B7-B7-B7 (GSC7056, SEQ ID NO: 55) digested with the same enzymes and treated with CIP. After miniprep screening, the midiprep pGLEX41_HC-I4-B7-PTCRA-B7 was prepared. It received the batch number GSC11204 and was confirmed by sequencing (SEQ ID NO: 93).

### Modifications of Charged Residues in PTCRA Transmembrane Domain

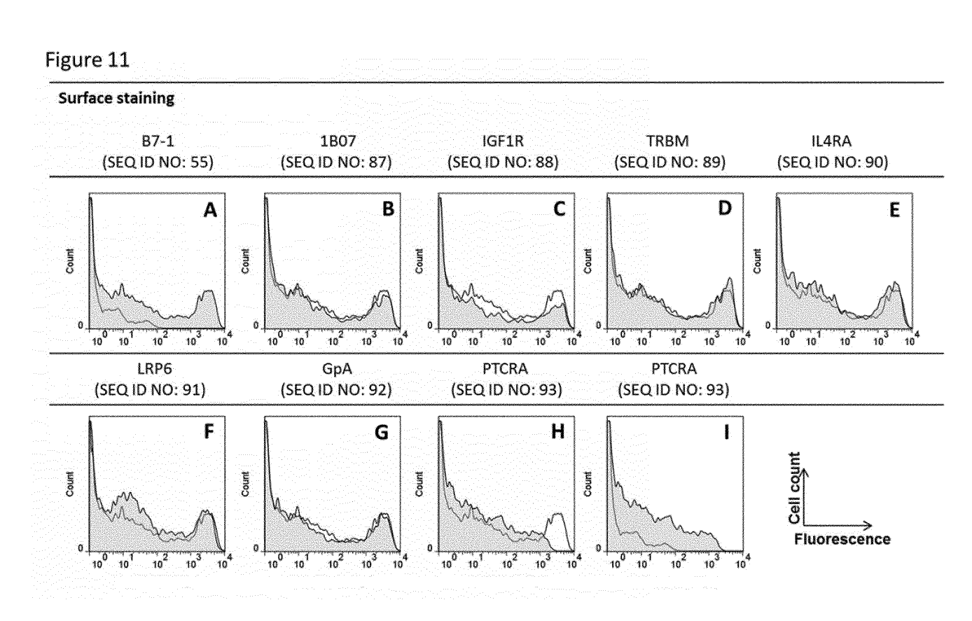

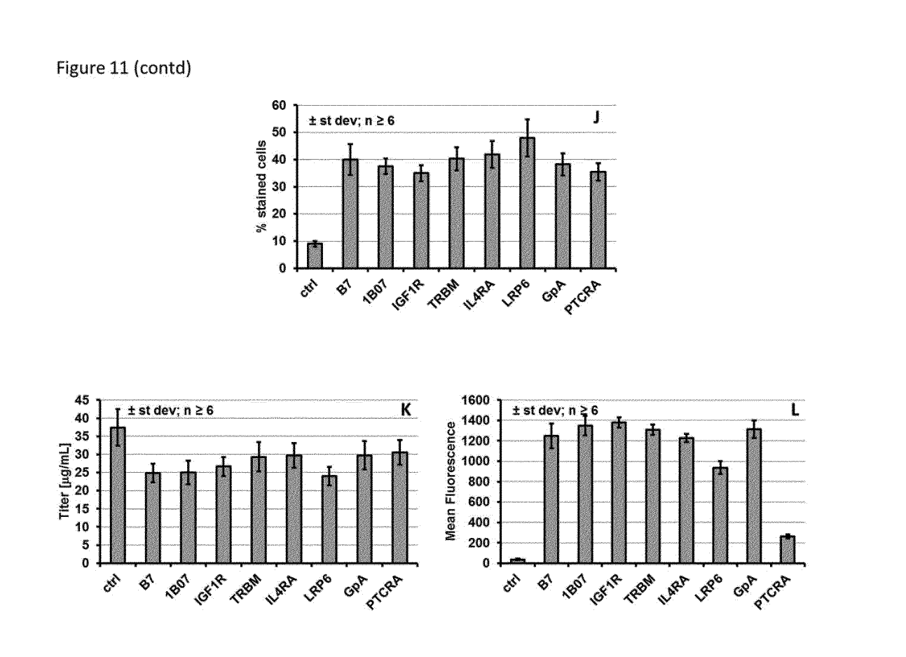

The PTCRA transmembrane domain is 21 amino acids long, 3 of which being charged residues. The presence of these charged residues might be an explanation for the relative poor performance of this particular transmembrane domain in the surface display system (see FIG. 11 in Example 4). A mutation of these residues to the hydrophobic amino acid valine might thus be beneficial in term of surface display.

TABLE 8
Mutations of charged residues in PTCRA transmembrane domain: sequences,
primers and DNA batch numbers.

| | | | | Primer |
|---|---|---|---|---|
| | Transmembrane | | Primer 1^{st} | pair 2^{nd} |
| | domain | SEQ ID | round | round |
| Construct name | sequence | NO | PCR | PCR |
| pGLEX41_HC-I4-B7-PTCRA_R8V- | ALWLGVLVLLLFKLLLFDLLL | 187 | GlnPr2382 | GlnPr2383 |
| B7 | | | (SEQ ID | (SEQ ID |
| | | | NO: 225) | NO: 226) |
| pGLEX41_HC-I4-B7-PTCRA_K13V- | ALWLGVLRLLLFVLLLFDLLL | 188 | GlnPr2380 | GlnPr2384 |
| B7 | | | (SEQ ID | (SEQ ID |
| | | | NO: 223) | NO: 227) |
| pGLEX41_HC-I4-B7-PTCRA_D18V- | ALWLGVLRLLLFKLLLFVLLL | 189 | GlnPr2380 | GlnPr2385 |
| B7 | | | (SEQ ID | (SEQ ID |
| | | | NO: 223) | NO: 228) |
| pGLEX41_HC-I4-B7-PTCRA_R8V_ | ALWLGVLVLLLFVLLLFDLLL | 190 | GlnPr2382 | GlnPr2386 |
| K13V-B7 | | | (SEQ ID | (SEQ ID |
| | | | NO: 225) | NO: 229) |
| pGLEX41_HC-I4-B7-PTCRA_R8V_ | ALWLGVLVLLLFKLLLFVLLL | 191 | GlnPr2382 | GlnPr2387 |
| D18V-B7 | | | (SEQ ID | (SEQ ID |
| | | | NO: 225) | NO: 230) |
| pGLEX41_HC-I4-B7-PTCRA_K13V_ | ALWLGVLRLLLFVLLLFVLLL | 192 | GlnPr2380 | GlnPr2388 |
| D18V-B7 | | | (SEQ ID | (SEQ ID |
| | | | NO: 223) | NO: 231) |
| pGLEX41_HC-I4-B7-PTCRA_R8V_ | ALWLGVLVLLLFVLLLFVLLL | 193 | GlnPr2382 | GlnPr2389 |
| K13V_D18V-B7 | | | (SEQ ID | (SEQ ID |
| | | | NO: 225) | NO: 232) |

All possible combinations of non-mutated and mutated charged residues in PTCRA transmembrane domain were generated by two step PCRs using pGLEX41_HC-I4-B7-B7-B7 (GSC7056, SEQ ID NO: 55) as template, with GlnPr1516 (SEQ ID NO: 9) as forward primer and the reverse primer described in Table 8 for the different constructs. After the 1^{st} round of PCR, PCR products were cleaned up and a second round of PCR was performed with GlnPr1516 (SEQ ID NO: 9) as forward primer and the reverse primer described in Table 8. The resulting PCR products were cleaned up, digested with ClaI and Age I and ligated in the backbone pGLEX41_HC-I4-B7-B7-B7 (GSC7056, SEQ ID NO: 55) digested with the same enzymes and treated with CIP. After miniprep screening, the positive constructs were prepared at midiprep scale and were confirmed by sequencing. The batch numbers of the respective constructs are the following:

| | |
|---|---|
| pGLEX41_HC-I4-B7-PTCRA_R8V-B7 | GSC11216 |
| | (SEQ ID NO: 94) |
| pGLEX41_HC-I4-B7-PTCRA_K13V-B7 | GSC11203 |
| | (SEQ ID NO: 95) |
| pGLEX41_HC-I4-B7-PTCRA_D18V-B7 | GSC11211 |
| | (SEQ ID NO: 96) |
| pGLEX41_HC-I4-B7-PTCRA_R8V_K13V-B7 | GSC11212 |
| | (SEQ ID NO: 97) |
| pGLEX41_HC-I4-B7-PTCRA_R8V_D18V-B7 | GSC11398 |
| | (SEQ ID NO: 98) |
| pGLEX41_HC-I4-B7-PTCRA_K13V_D18V-B7 | GSC11208 |
| | (SEQ ID NO: 99) |
| pGLEX41_HC-I4-B7- | GSC11291 |
| PTCRA_R8V_K13V_D18V-B7 | (SEQ ID NO: 100) |

Replacement of B7-1 Transmembrane Domain by Transmembrane Domains of Different Lengths Containing Charged and/or Polar Residues

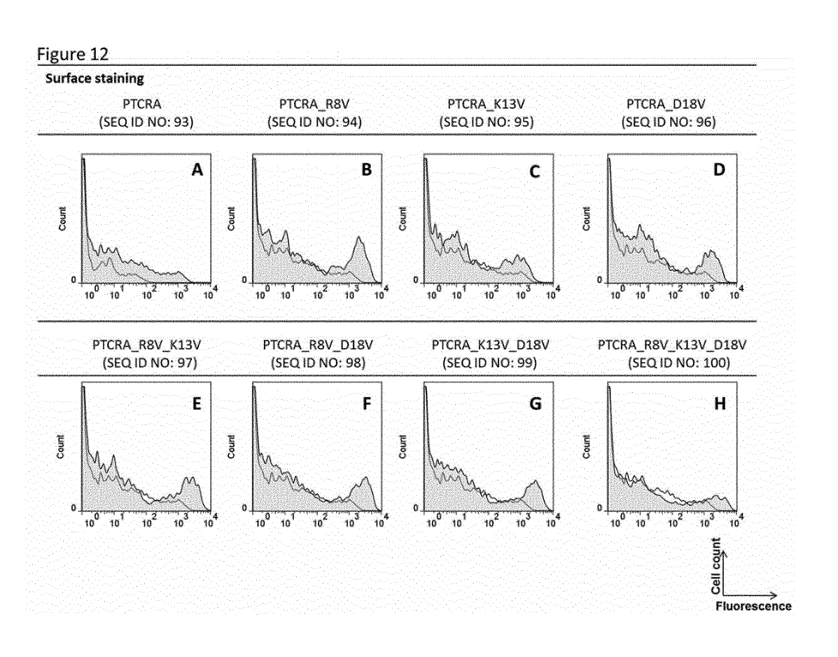

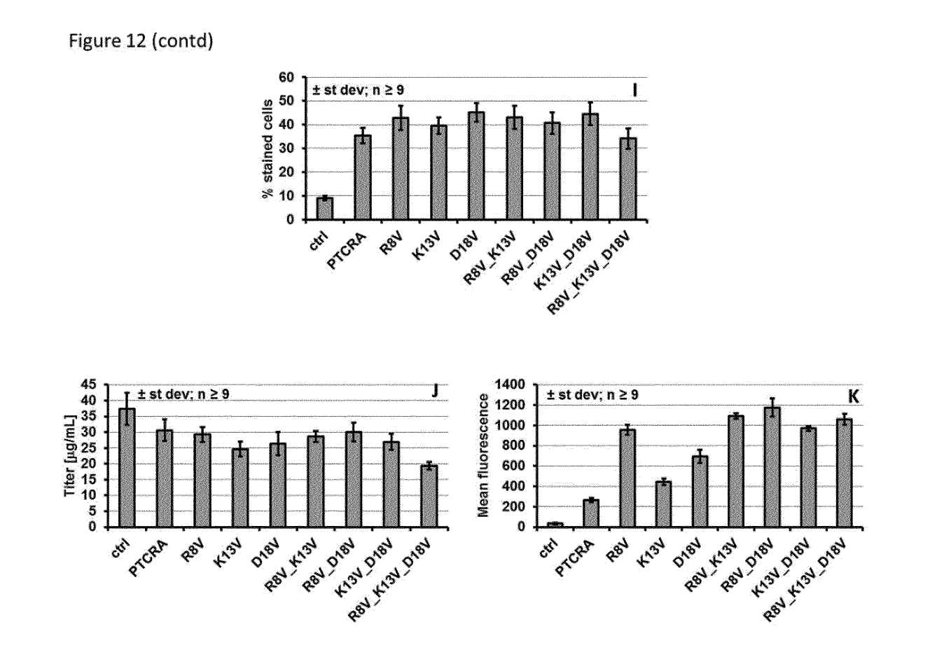

As the presence of charged and residues in the transmembrane domains does not abrogate the surface display (see FIG. 12 in Example 4), the length of the transmembrane domain was assessed using transmembrane domains shorter or longer than B7-1 transmembrane domain and containing this times one or more charged and/or polar residues.

The sequences and characteristics of these transmembrane domains are summarized in the Table 9.

TABLE 9
Transmembrane domains of different length containing charged and/or
polar residues: characteristics and sequences.

| Trans- | | | | | | SEQ |
|---|---|---|---|---|---|---|
| membrane | | Hydro- | | | | ID |
| domian | Length | phobic | Polar | Charged | Sequence | NO |
| B7 | 22 | 20 | 2 | 0 | TLVLFGAGFGAVITVVVIVVII | 173 |
| IL3RB | 17 | 16 | 1 | 0 | VLALIVIFLTIAVLLAL | 196 |
| FCERA | 19 | 17 | 1 | 1 | FFIPLLVVILFAVDTGLFI | 194 |
| KI2L2 | 19 | 17 | 2 | 0 | ILIGTSVVIILFILLFFLL | 195 |
| CD3E | 26 | 18 | 7 | 1 | VMSVATIVIVDICITGGLLLLVYYWS | 197 |
| ITA2B | 26 | 25 | 1 | 0 | AIPIWWVLVGVLGGLLLLTILVLAMW | 198 |
| CD28 | 27 | 23 | 4 | 0 | FWVLVVVGGVLACYSLLVTVAFIIFWV | 199 |
| M1M2 | 27 | 16 | 10 | 1 | LWTTITIFITLFLLSVCYSATVTFFKV | 200 |

TABLE 10
GeneArt constructs used for the cloning of transmembrane domains
with different lengths containing charged and/or polar residues.

| Trans- | | |
|---|---|---|
| membrane | | |
| domain | GeneArt Seq name | GeneArt SEQ ID NO |
| IL3RB | GeneArt_Seq 88 | 270 |
| FCERA | GeneArt_Seq 89 | 271 |
| KI2L2 | GeneArt_Seq 90 | 272 |
| CD3E | GeneArt_Seq 91 | 273 |
| ITA2B | GeneArt_Seq 92 | 274 |
| CD28 | GeneArt_Seq 93 | 275 |
| M1M2 | GeneArt_Seq 80 | 262 |

The different transmembrane domains were ordered from GeneArt with the names indicated in the Table 10. Once received, GeneArt constructs were resuspended in water and then digested with AgeI and ClaI. The resulting inserts were ligated in the backbone pGLEX41_HC-I4-B7-B7-B7 (GSC7056, SEQ ID NO: 55) digested with the same enzymes and treated with CIP. After miniprep screening, midipreps were produced and received the following batch numbers:

| | |
|---|---|
| pGLEX41-GBR200HC-I4-B7-FCERA-B7 | GSC11297 (SEQ ID NO: 101) |
| pGLEX41-GBR200HC-I4-B7-KI2L2-B7 | GSC11298 (SEQ ID NO: 102) |
| pGLEX41-GBR200HC-I4-B7-IL3RB-B7 | GSC11393 (SEQ ID NO: 103) |
| pGLEX41-GBR200HC-I4-B7-CD3E-B7 | GSC11394 (SEQ ID NO: 104) |
| pGLEX41-GBR200HC-I4-B7-ITA2B-B7 | GSC11395 (SEQ ID NO: 105) |
| pGLEX41-GBR200HC-I4-B7-CD28-B7 | GSC11396 (SEQ ID NO: 106) |
| pGLEX41-GBR200HC-I4-B7-M1M2-B7 | GSC11389 (SEQ ID NO: 107) |

### Modification of B7-1 Transmembrane Domain Length

In order to assess the impact of the transmembrane length on surface display, different variants of B7-1 transmembrane domains were generated by removing or adding hydrophobic residues in the middle of B7-1 transmembrane domain sequence.

TABLE 11
Modification of B7-1 transmembrane domain length: sequence and
primer pairs.

| Transmenbrane | | SEQ ID | |
|---|---|---|---|
| domain | Sequence | NO | Primers |
| B7 (18) | TLVLFGAGFGATVIVVII | 201 | GlnPr2395 (SEQ |
| | | | ID NO: 238)/ |
| | | | GlnPr2394 (SEQ |
| | | | ID NO:237) |
| B7 (20) | TLVLFGAGFGATVVVIVVII | 202 | GlnPr2392 (SEQ |
| | | | ID NO: 235)/ |
| | | | GlnPr2393 (SEQ |
| | | | ID NO:236) |
| B7 (24) | TLVLFGAGFGAVVIVTVVVIVVII | 203 | GlnPr2396 (SEQ |
| | | | ID NO: 239)/ |
| | | | GlnPr2397 (SEQ |
| | | | ID NO:240) |
| B7 (26) | TLVLFGAGFGAVVIVVVTVVVIVVII | 204 | GlnPr2398 (SEQ |
| | | | ID NO: 241)/ |
| | | | GlnPr2399 (SEQ |
| | | | ID NO:242) |

For this purpose, primer annealing was performed. For each variant of B7-1 transmembrane domains, primers were ordered that anneal in their 3′ ends. The primer pairs used for each construct are described in Table 11. The primers were allowed to anneal and then the sequence was completed by PCR. After clean-up, the resulting PCR products were either directly digested with SfoI and AgeI and cloned into pGLEX41_HC-I4-B7-B7-B7 (GSC7056, SEQ ID NO: 55) backbone digested with the same enzymes and treated with CIP, or ligated into the pCR-blunt (Zero Blunt® PCR Cloning Kit, Invitrogen, LifeTechnologies) shuttle vector (as the annealing products are very short, the direct ligation did not work for every product). In the 2^{nd} case, minipreps were extracted from colonies obtained after ligation of the annealed product in pCR-blunt (Zero Blunt® PCR Cloning Kit, Invitrogen, LifeTechnologies). After sequence confirmation, the positive minipreps were digested with SfoI and AgeI and the obtained fragments were ligated cloned into pGLEX41_HC-I4-B7-B7-B7 (GSC7056, SEQ ID NO: 55) backbone digested with the same enzymes and treated with CIP. After miniprep screening, midipreps were prepared for each constructs. A GSC batch number was attributed to these midipreps as described below and the preps were confirmed by sequencing.

| | |
|---|---|
| pGLEX41-GBR200HC-I4-B7-B7(18)-B7 | GSC11677 (SEQ ID NO: 108) |
| pGLEX41-GBR200HC-I4-B7-B7(20)-B7 | GSC11679 (SEQ ID NO: 109) |
| pGLEX41-GBR200HC-I4-B7-B7(24)-B7 | GSC11484 (SEQ ID NO: 110) |
| pGLEX41-GBR200HC-I4-B7-B7(26)-B7 | GSC11292 (SEQ ID NO: 111) |

### Modification of Cytosolic Tail in B7-1 Transmembrane Region

As the cytosolic tail (with or without an ER exportation signal) may have an impact on surface display, several constructs were designed with modified cytosolic tails. The sequences and characteristics of these cytosolic tails are summarized in the Table 12.

TABLE 12
Cytosolic tails: characteristics and sequences.

| | ER | export | SEQ |
|---|---|---|---|
| Cytosolic tail | signal | Sequence | ID NO. |
| B7 | Yes | KCFCKHRSCFRRNEASRETNNSLTFGPEEALAEQT | 218 |
| | | VFL | |
| M1M2 | ? | KWIFSSVVDLKQTIIPDYRNMIGQGA | 207 |
| 6His | No | ASAHHHHHH | 205 |
| KCFCK | No | KCFCK | 206 |
| ERGIC53 | Yes | RSQQEAAAKKFF | 208 |
| ERGIC53_noER | No | RSQQEAAAKKWW | 209 |
| GAT1 | Yes | MFLTLKGSLKQRLQVMIQPSEDIVRPENGPEQPQA | 210 |
| | | GSSASKEAYI | |
| GAT1_noER | No | MFLTLKGS | 211 |
| AGTR2 | Yes | CFVGNRFQQKLRSVFRVPITWLQGKRETMSCRKSS | 212 |
| | | SLREMDTFVS | |
| AGTR2_noER | No | CFVGNRFQQKLRSVFRVPITWLQGKRETMSCRKSS | 213 |
| | | SLR | |
| V1BR | Yes | NSHLLPRPLRHLACCGGPQPRMRRRLSDGSLSSRH | 214 |
| | | TTLLTRSSCPATLSLSLSLTLSGRPRPEESPRDLELA | |
| | | DGEGTAETIIF | |
| V1BR_noER | No | NSHEESPRDLELADGEGTAETIIF | 215 |
| VGLG | Yes | CIKLKHTKKRQIYTDIEMNRLGK | 216 |
| VGLG_noER | No | CIKLKHTKKRQIAAAAAANRLGK | 217 |

Different cloning strategies were applied according to the length of the different constructs. The first constructs were generated by PCR amplification of the cTNT intron 4 with B7-1 extracellular and transmembrane domains with primers modifying the cytosolic tail.

TABLE 13
Primer pairs and enzymes used for cytosolic tail modification.

| Cytosolic tail | Primer pair | Enzymes |
|---|---|---|
| 6His | GlnPr2377 (SEQ ID NO: 220)/ | HindIII/BstBI |
| | GlnPr 1516 (SEQ ID NO: 9) | |
| KCFCK | GlnPr 2376 (SEQ ID NO: 219)/ | HindIII/BstBI |
| | GlnPr 1516 (SEQ ID NO: 9) | |
| GAT1_noER | GlnPr 2415 (SEQ ID NO: 258)/ | ClaI/BstBI |
| | GlnPr 1516 (SEQ ID NO: 9) | |

The primers pairs used are described in the Table 13 and the template used was pGLEX41_HC-I4-B7-B7-B7 (GSC7056, SEQ ID NO: 55). The purified PCR products were digested with the enzymes described in Table 13 and cloned in the backbone pGLEX41_HC-I4-B7-B7-B7 (GSC7056, SEQ ID NO: 55) digested with the same enzymes and treated with CIP. After miniprep screening, midipreps were produced which received the following batch number:

| | |
|---|---|
| pGLEX41-GBR200HC-I4-B7-B7-6His | GSC11207 (SEQ ID NO: 112) |
| pGLEX41-GBR200HC-I4-B7-B7- | GSC11209 (SEQ ID NO: 113) |
| KCFCK | |
| pGLEX41-GBR200HC-I4-B7-B7- | GSC11483 (SEQ ID NO: 118) |
| GAT1noER | |

All midipreps were confirmed by sequencing.

In order to fuse the M1M2 cytosolic tail to the B7-1 transmembrane region, a fusion PCR was performed. The cTNT intron 4 with the B7-1 extracellular and transmembrane domains were amplified with GlnPr1516 (SEQ ID NO: 9) and GlnPr2391 (SEQ ID NO: 234) using pGLEX41_HC-I4-B7-B7-B7 (GSC7056, SEQ ID NO: 55) as template. The M1M2 cytosolic tail with the SV40 poly(A) signal was amplified with the primers GlnPr2390 (SEQ ID NO: 233) and GlnPr1650 (SEQ ID NO: 19) using pGLEX41_HC-I4-M1M2-M1M2-M1M2 (GSC3899, SEQ ID NO: 36) as template. After clean-up, both PCR products were fused in a 3^{rd} PCR using GlnPr1516 (SEQ ID NO: 9) and GlnPr1650 (SEQ ID NO: 19) as primers. The resulting PCR product was cleaned up, digested with SfoI and BstBI and ligated in pGLEX41_HC-I4-B7-B7-B7 (GSC7056, SEQ ID NO: 55) digested with the same enzymes and treated with CIP. After miniprep screening, the midiprep pGLEX41_HC-I4-B7-B7-M1M2 was produced, received the batch number GSC11758 and was confirmed by sequencing (SEQ ID NO: 114).

For some constructs, a strategy of primer annealing was chosen. For each cytosolic tail, primers were ordered that anneal in their 3′ ends. The primer pairs used for each construct are described in Table 14. The primers were allowed to anneal and then the sequence was completed by PCR. After clean-up, the resulting PCR products were either directly digested with SfoI and BstBI and cloned into pGLEX41_HC-I4-B7-B7-B7 (GSC7056, SEQ ID NO: 55) backbone digested with the same enzymes and treated with CIP, or ligated into the pCR-blunt shuttle vector (Zero Blunt® PCR Cloning Kit, Invitrogen, LifeTechnologies) (as the annealing products are very short, the direct ligation did not work for every product). In the 2^{nd} case, minipreps were extracted from colonies obtained after ligation of the annealed product in pCR-blunt (Zero Blunt® PCR Cloning Kit, Invitrogen, LifeTechnologies). After sequence confirmation, the positive minipreps were digested with SfoI and BstBI and the obtained fragments were ligated into pGLEX41_HC-I4-B7-B7-B7 (GSC7056, SEQ ID NO: 55) backbone digested with the same enzymes and treated with CIP. After miniprep screening, midipreps were prepared for each constructs. A GSC batch number was attributed to theses midipreps as described in Table 14 and the preps were confirmed by sequencing.

TABLE 14
Primer pairs used for primer annealing for cytosolic
tail modification and DNA batch numbers.

| | | Midiprep |
|---|---|---|
| Cytosolic tail | Primers | Batch number |
| ERGIC53 | GlnPr2412 (SEQ ID NO: 255)/ | GSC11764 |
| | GlnPr2413 (SEQ ID NO: 256) | (SEQ ID NO: 115) |
| ERGIC53_noER | GlnPr2412 (SEQ ID NO: 255)/ | GSC11678 |
| | GlnPr2414 (SEQ ID NO: 257) | (SEQ ID NO: 116) |

Finally, for the last constructs, the sequences were ordered from GeneArt with the sequence names described in the Table 15. Once received, GeneArt constructs were resuspended in water and then digested with SfoI and BstBI. The resulting inserts were ligated in the backbone pGLEX41_HC-I4-B7-B7-B7 (GSC7056, SEQ ID NO: 55) digested with the same enzymes and treated with CIP. After miniprep screening, midipreps were produced and received the batch numbers detailed in the following table.

TABLE 15
GeneArt sequences ordered for cytosolic
tail modification and DNA batch numbers.

| | | Geneart | Midiprep |
|---|---|---|---|
| Cytosolic tail | GeneArt Seq | SEQ ID NO | batch number |
| GAT1 | GeneArt_Seq95 | 277 | GSC11759 |
| | | | (SEQ ID NO: 117) |
| AGTR2 | GeneArt_Seq 96 | 278 | GSC11760 |
| | | | (SEQ ID NO: 119) |
| AGTR2_noER | GeneArt_Seq 97 | 279 | GSC11761 |
| | | | (SEQ ID NO: 120) |
| V1BR | GeneArt_Seq 98 | 280 | GSC11762 |
| | | | (SEQ ID NO: 121) |
| V1BR_noER | GeneArt_Seq 99 | 281 | GSC11765 |
| | | | (SEQ ID NO: 122) |
| VGLG | GeneArt_Seq 100 | 282 | GSC11766 |
| | | | (SEQ ID NO: 123) |
| VGLG_noER | GeneArt_Seq 101 | 283 | GSC11763 |
| | | | (SEQ ID NO: 124) |

In order to further evaluate the impact of the B7-1 cytosolic tail, the cytosolic tail of M1M2 transmembrane domain was replaced by the B7-1 cytosolic tail. For this purpose, the M1M2 transmembrane domain with the B7-1 cytosolic tail was ordered from GeneArt as GeneArt_Seq82 (SEQ ID NO: 264). The GeneArt construct was digested with ClaI and BstBI and the insert was ligated in the backbone pGLEX41_HC-I4-B7-B7-B7 (GSC7056, SEQ ID NO: 55) digested with the same enzymes and treated with CIP. After miniprep screening, the midiprep pGLEX41_HC-M1M2-M1M2-B7 was produced, received the plasmid batch number GSC11294 and was confirmed by sequencing (SEQ ID NO: 125).

### Cloning of the Vector for the Expression of the Light Chain of an IgG1 Antibody

The cloning vector provided from GeneArt carrying the light chain insert (0704970pGA4) was used as a template for PCR using the primers GlnPr501 (SEQ ID NO: 4) and GlnPr502 (SEQ ID NO:5). These primers amplify the open reading frame of the light chain and add a HindIII restriction site 5′ and an XbaI restriction site 3′ to the open reading frame. The amplicon was digested with the XbaI and HindIII and was ligated in a shuttle vector cut using XbaI and HindIII. After miniprep screening, a gigaprep was produced, which was confirmed by sequencing and digestion.

The coding region of the light chain of an antibody of the IgG1 format was cut from this gigaprep with XbaI and HindIII, and cloned into the backbone of the plasmid pGLEX41 (GSC281, SEQ ID NO: 304) opened with the same enzymes and treated with CIP. After miniprep screening, a maxiprep of pGLEX41_LC was produced, received the batch number GSC315 (SEQ ID NO: 294) and was confirmed by sequencing. Subsequently, several batches of the same DNA were produced at midiprep or gigaprep level and received the batch numbers GSC315a, GSC315b, GSC315c, GSC315d and GSC315e (as all these preps were confirmed by sequencing they will be referred to in the following as GSC315 plasmid (SEQ ID NO: 294)).

### Cloning of the Vector for the Expression of the Light Chain of an IgG4 Antibody

The sequence coding for the IgG4 heavy chain was generated at Glenmark and was received in a shuttle vector. The IgG4 heavy chain coding sequence was amplified by PCR with the primers GlnPr1488 (SEQ ID NO: 328) and GlnPr1452 (SEQ ID NO:327). After purification, the resulting PCR product was re-amplified with the primers GlnPr1494 (SEQ ID NO: 260) and GlnPr1452 (SEQ ID NO: 327). These two rounds of PCR modified the leader peptide and added restriction sites at both ends of the coding sequence. The resulting PCR product was purified and cloned in the shuttle vector pCR-blunt (Zero Blunt® PCR Cloning Kit, Invitrogen, LifeTechnologies). After miniprep screening, a positive clone was digested with SpeI and ClaI to extract the IgG4 heavy chain coding sequence. The resulting insert was ligated in pGLEX41 (GSC281, SEQ ID NO: 304) digested with NheI and BstBI and treated with CIP. After miniprep screening, the gigaprep pGLEX41_IgG4-HC was produced, received the plasmid batch number GSB59 and was confirmed by sequencing (SEQ ID NO: 79).

Cloning of the Vector for the Expression of the Heavy Chain of an IgG4 Antibody with Alternate Splicing Allowing the Expression of the B7-1 Transmembrane Region

For the addition of the alternate splicing cassette for membrane-bound IgG4 expression, the coding sequence for IgG4 heavy chain was amplified by PCR with the primers GlnPr1494 (SEQ ID NO: 260) and GlnPr2294 (SEQ ID NO: 261) using pGLEX41_IgG4-HC (GSB59, SEQ ID NO: 79) as template. The resulting PCR product was cleaned-up, digested with SpeI and HindIII and ligated into the pGLEX41_HC-I4-B7-B7-B7 (GSC7056, SEQ ID NO: 55) backbone digested with NheI and HindIII and treated with CIP. After miniprep screening, the midiprep pGLEX41_IgG4-HC-B7-B7-B7 was prepared which received the plasmid batch number GSC11218 and was confirmed by sequencing (SEQ ID NO: 80).

### Cloning of the Vector for the Expression of the Light Chain of an IgG4 Antibody

The sequence coding for the IgG4 light chain was generated at Glenmark and received in a shuttle vector. Using a single round of PCR with three different primers GlnPr1487 (SEQ ID NO:325), GlnPr1491 (SEQ ID NO: 326) and GlnPr1494 (SEQ ID NO:260), the leader peptide was exchanged and restriction sites were added at both ends of the coding sequence. The PCR product was digested with SpeI and ClaI and ligated in the pGLEX41 (GSC281, SEQ ID NO: 304) backbone opened with NheI and BstBI and treated with CIP. After miniprep screening, the midiprep pGLEX41_IgG4-LC was produced, received the plasmid batch number GSC3704 and was confirmed by sequencing (SEQ ID NO: 319).

### Cloning of the Vector for the Expression of Secreted Heavy Chain of a BEAT® Bispecific Antibody

The heavy chain of the bispecific antibody was generated at Glenmark as part of the bispecific BEAT® format. Using two rounds of PCR, a kozak sequence, a signal peptide and flanking restriction sites were added. In the first round, the template was amplified using primers GlnPr1726 (SEQ ID NO: 24) and GlnPr1500 (SEQ ID NO: 7). The PCR product was purified and used as template for a second round of PCR using primers GlnPr1500 (SEQ ID NO: 7) and GlnPr1725 (SEQ ID NO: 23). The resulting amplicon was purified and cut using the restriction enzymes SpeI and ClaI. This insert was cloned into the backbone pGLEX41 (GSC281, SEQ ID NO: 304), opened using NheI and BstBI and treated with CIP. After miniprep screening, the pGLEX41_BEAT-HC-His was produced in midiprep scale and received the batch number GSC5607 (SEQ ID NO: 296). The sequencing control and the restriction digest confirmed the plasmid.

This plasmid was used as template for a modifying PCR using primers GlnPr1725 (SEQ ID NO: 23) and GlnPr1760 (SEQ ID NO: 27) for removal of the His-tag from the template sequence. The amplicon was digested with SpeI and ClaI and cloned in the backbone of the vector pGLEX41 (GSC281, SEQ ID NO: 304), opened using the restriction enzymes NheI and BstBI and treated with CIP. After miniprep screening, the midiprep pGLEX41_BEAT-HC was given the plasmid batch number GSC5644 and was confirmed by sequencing (SEQ ID NO: 49).

### Cloning of the Alternate Splicing Expression Vector for Membrane Displayed Heavy Chain of a Bispecific BEAT® Antibody

The template vector coding for the BEAT heavy chain (GSC5644, SEQ ID NO: 49) was amplified using the primers GlnPr1800 (SEQ ID NO: 29) and GlnPr1725 (SEQ ID NO: 23). The purified product was digested using the restriction enzymes SpeI and HindIII and cloned in the vector pGLEX41_HC-I4-M1M2-M1M2-M1M2 (GSC3899, SEQ ID NO: 36) opened using the enzymes NheI and HindIII and treated with CIP. After miniprep screening, the maxiprep pGLEX41_BEAT-HC-I4-M1M2-M1M2-M1M2, received the plasmid batch number GSC5537 (SEQ ID NO: 50) and was confirmed by restriction digest and sequencing.

In order to use the B7-1 transmembrane region for surface display, the insert of the construct pGLEX41_BEAT-HC-I4-M1M2-M1M2-M1M2 (GSC5537, SEQ ID NO: 50) was digested with SacI and HindIII and recloned in pGLEX41_HC-I4-B7-B7-B7 (GSC7056, SEQ ID NO: 55) digested with the same enzymes and treated with CIP. After miniprep screening, the maxiprep pGLEX41_BEAT-HC-I4-B7-B7-B7 was produced, received the plasmid batch number GSC10488 and was confirmed by sequencing (SEQ ID NO: 321).

Cloning of the Expression Vector for Secreted scFv-Fc of a Bispecific Antibody

The scFv-Fc sequence was developed in-house as part of the bispecific BEAT® format. Amplification was performed using primers GlnPr1690 (SEQ ID NO: 22), GlnPr1689 (SEQ ID NO: 21) and GlnPr1502 (SEQ ID NO: 8). These primers add a kozak sequence, a signal peptide and restriction sites to the amplicon. The PCR product was re-amplified using primers GlnPr1689 (SEQ ID NO: 21) and GlnPr1502 (SEQ ID NO: 8) and the resulting amplicon was digested using NheI and ClaI. This insert fragment was ligated with the backbone pGLEX41 (GSC281, SEQ ID NO: 304) cut using NheI and BstBI and treated with CIP. After miniprep screening, the midiprep pGLEX41_scFv-Fc with the batch number GSC5608 (SEQ ID NO: 51) was produced and confirmed by restriction digest and sequencing.

Cloning of the Alternate Splicing Expression Vector for Membrane Displayed scFv-Fc of a Bispecific Antibody

The template vector coding for the scFv (GSC5608, SEQ ID NO: 51) was amplified using the primers GlnPr1801 (SEQ ID NO: 30) and GlnPr1689 (SEQ ID NO:21). The purified product was digested with NheI and HindIII and cloned in the vector pGLEX41_HC-I4-M1M2-M1M2-M1M2 (GSC3899, SEQ ID NO: 36) opened using the same enzymes and treated with CIP. After miniprep screening, the midiprep pGLEX41_scFv-Fc-I4-M1M2-M1M2-M1M2 with the plasmid batch number GSC5538 (SEQ ID NO: 52) was produced and confirmed by restriction digest and sequencing.

In order to use the B7-1 transmembrane region for surface display, the insert of the construct pGLEX41_scFv-Fc-I4-M1M2-M1M2-M1M2 (GSC5537, SEQ ID NO: 50) was digested with NheI and HindIII and recloned in pGLEX41_HC-I4-B7-B7-B7 (GSC7056, SEQ ID NO: 55) digested with the same enzymes and treated with CIP. After miniprep screening, the maxiprep pGLEX41_scFv-Fc-I4-B7-B7-B7 was produced, received the plasmid batch number GSC10487 and was confirmed by sequencing (SEQ ID NO: 323).

### Cloning of the Expression Vector for the Light Chain of a Bispecific Antibody

The light chain sequence was developed in-house as part of the bispecific BEAT® format. Amplification was performed using primers GlnPr1689 (SEQ ID NO: 21), GlnPr1727 (SEQ ID NO: 25) and GlnPr1437 (SEQ ID NO:6). These primers add a signal peptide, a Kozak sequence and flanking restriction sites to the amplicon. The amplicon was purified and digested using the restriction enzymes ClaI and NheI and cloned in the backbone of a shuttle vector, opened with NheI and ClaI and treated with CIP. After miniprep screening, a maxiprep was prepared and verified by sequencing. This shuttle vector coding for the light chain of the bispecific construct was digested using the restriction enzymes NheI and ClaI and the insert was cloned in the vector pGLEX41 (GSC281, SEQ ID NO: 304), opened with NheI and BstBI and treated with CIPed. After miniprep screening, the midiprep of pGLEX41_BEAT-LC was prepared (plasmid batch number GSC5540, SEQ ID NO: 302) and the plasmid was confirmed by restriction digest and sequencing.

### Example 2: Surface Display and Specific Detection of Translated Product

### Introduction

Example 1 described the cloning of alternate splicing constructs leading to expression of two different mRNAs from the same DNA template, coding either for a secreted protein or the same protein with a C-terminal transmembrane region (TM). As detailed in the definition section, a transmembrane region comprises an optional linker, a transmembrane domain and an optional cytoplasmic tail. These constructs were transfected in CHO-S cells in order to determine whether this technology could be used for displaying a fraction of the protein on the cell membrane, while maintaining an efficient secretion of the target protein. Three different proteins were used in this experiment: An antibody of the IgG1 subclass, an antibody of the IgG4 subclass and a bispecific antibody of the BEAT® format.

### Material and Methods

### Transfections

Suspension CHO-S cells were transfected with the expression vectors using polyethyleneimine (JetPEI®, Polyplus-transfection, Illkirch, France) in 50 ml bioreactor tube (Tubespins, TPP) format. For this purpose, exponential growing cells were seeded at a density of 2 E6 cells/mL in 5 mL of OptiMEM medium (#31985-047, Invitrogen) or CD-CHO Medium (#10743-011, Life Technologies). A JetPEI®:DNA complex was added to the cells in a weight ratio of 3 (μg/μg). Final DNA concentration in the cell suspension was 2.5 μg/mL. After 5 hours incubation at 37° C. under shaking (200 rpm), 5 mL of fresh culture medium were added to the cell suspension. Then the cells were incubated on a shaken platform at 37° C., 5% CO₂ and 80% humidity.

### Surface Staining

Surface staining of the cells was performed on day 1 after transfection. A total of 1 E5 cells were harvested and transferred to a round bottom well of a 96 well plate. The cells were washed twice with washing buffer (2% FBS in PBS) and then resuspended in 100 μL washing buffer containing the detection antibody. The specific detection of the kappa light chain was performed using a mouse anti-human kappa light chain APC labelled antibody (#561323, BD Pharmingen), excited by a red laser (640 nm) and detected in the spectral range 661/19 nm. Both, heavy chain and scFv-Fc, were detected using PE conjugated goat anti-human Fc gamma specific antibody (#12-4998-82, eBioscience) excited by a blue laser (488 nm) and detected in the spectral range 583/26 nm and the scFv-Fc was detected specifically using a FITC-labelled Protein A (#P5145, Sigma) excited at 488 nm by a blue laser and detected in the spectral range 525/30. After 20 min incubation in the dark at room temperature, the cells were washed once in washing buffer and resuspended in 200 μL of washing buffer for flow cytometric analysis. The cells were analysed with a Guava flow cytometer (Merck Millipore) or with a FACSCalibur (Becton Dickinson)

The transient expression level of secreted molecules was measured on day 4-6 after transfection using the Octet QK instrument (Fortebio, Menlo Park, Calif.) and protein A biosensors.

### Results

For IgG1 antibody expression, CHO-S cells were transfected as described in the Material and Methods section, using the expression vector pGLEX41_LC (SEQ ID NO: 294) expressing the light chain and a second vector named pGLEX41_HC-I4-B7-B7-B7 (SEQ ID NO: 55), pGLEX41_HC-I4-M1M2-M1M2-M1M2 (SEQ ID NO: 36), pGLEX41_HC-I4-PTCRA (SEQ ID NO: 40) or pGLEX41_HC-I4-M1M2-PTCRA-M1M2_corrected (SEQ ID NO: 284), respectively. As control, the non-splicing construct coding for secreted heavy chain was used (SEQ ID NO: 48).

After transfection, the cells were stained as described in Materials and Methods and analyzed by flow cytometry. The negative control expressing the secreted version of light chain and heavy chain (no alternate splicing of a transmembrane region) only yielded a low intensity of surface staining (see unfilled histogram in FIG. 2A and FIG. 2E for the percentage of stained cells) with a relatively high secretion level of IgG1 (see FIG. 2F). In contrast to this, cells transfected with constructs containing the M1M2 region (SEQ ID NO: 36) showed a significant positive population displaying the antibody on the cell membrane (see filled histogram in FIG. 2B). The exchange of the entire M1M2 transmembrane region by the PTCRA transmembrane region (see FIG. 2C; SEQ ID NO: 40), or the specific exchange of the transmembrane domain of M1M2 by the transmembrane domain of PTCRA (see FIG. 2D; SEQ ID NO: 284) led to a positive, but weakly stained population of cells. The murine B7-1 transmembrane region (see FIG. 2A; SEQ ID NO: 55) showed a surprisingly high level of staining with 40% of the population positive for membrane displayed IgG1.

Alternate splicing and hence, the membrane-bound expression of a fraction of the overall produced antibody seems to have a negative impact on the titers of secreted IgG. Nevertheless, the M1M2 transmembrane region (also in combination with the PTCRA transmembrane domain) and especially the B7-1 transmembrane region allowed up to 80% of the secretion level of the control construct without alternate splicing (see FIG. 2F).

For IgG4 antibody expression CHO-S cells were transfected as described in the Material and Methods section, using the expression vector pGLEX41_IgG4-LC (SEQ ID NO: 319) expressing the light chain and a second vector coding for the heavy chain with alternate splicing of the B7-1 transmembrane region for membrane display (SEQ ID NO: 80). As control, the non-splicing construct coding for secreted IgG4 heavy chain was used (SEQ ID NO: 79).

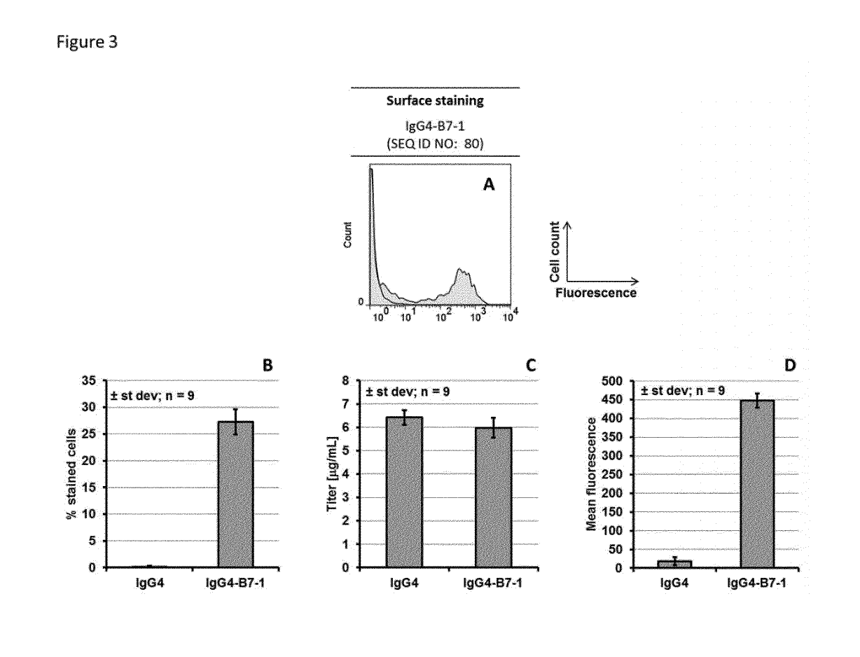

After transfection, the cells were stained as described in Materials and Methods and analyzed by flow cytometry. The negative control expressing the secreted version of light chain and heavy chain (without alternate splicing of a transmembrane region) only yielded a negative population (see FIG. 3A) with a substantial secretion level of IgG4 of approx. 6 μg/mL (see FIG. 3C). In contrast to this, cells transfected with constructs containing the B7-1 region (SEQ ID NO: 80) showed a significant positive population displaying the antibody on the cell membrane (see FIG. 3A for a histogram and FIG. 3B for the percentage of staining) while the expression level remained at a similar level with and without alternate splicing (see FIG. 3C).

To address whether a bispecific antibody could be displayed on the cell membrane via a transmembrane region (TM), the alternate splicing constructs described in Example 1 coding for bispecific antibodies were transiently expressed in CHO cells. The alternate splicing constructs code for a bispecific antibody format developed in-house and described in WO2012/131555 (termed “BEAT®”), which was used as a model protein in this example. The BEAT molecule is a trimer of an IgG heavy chain (“heavy chain”), a kappa light chain (“light chain”) and a scFv fused to a heavy chain constant region (“scFv-Fc”). The Protein A-binding site of the heavy chain was abrogated so that only the Fc-fragment of the scFv-Fc was able to bind to Protein A. The design of the molecule and the engineering of the heavy chain Protein A binding site allowed for specific detection of every subunit of the trimer. Display on the cell membrane was achieved by either transfecting the alternate splicing construct of either the heavy chain or the scFv-Fc or transfecting both alternate splicing constructs. Membrane bound BEAT bispecific antibody displayed on the surface of single cells was specifically detected by flow cytometry.

In the following, the vectors coding for the regular expression constructs will be shortened to “pHC” for pGLEX41_BEAT-HC (SEQ ID NO: 49), “pLC” for pGLEX41_BEAT-LC (SEQ ID NO: 302) and “pScFv-Fc” for pGLEX41_scFv-Fc (SEQ ID NO: 51). The alternate splicing construct adding a transmembrane region to the HC will be termed “pHC-M1M2” (pGLEX41_BEAT-HC-I4-M1M2-M1M2-M1M2; SEQ ID NO: 50) and the construct adding a transmembrane region to the scFv-Fc will be termed “pScFv-Fc-M1M2” (pGLEX41_scFv-Fc-I4-M1M2-M1M2-M1M2; SEQ ID NO: 52). The expression vectors coding for the three subunits of the BEAT antibody were co-transfected using the alternate splicing expression constructs or regular expression constructs in different combinations. Table 16 summarizes the transfections performed and the species expected to be displayed on the cell membrane.

TABLE 16
Transfection scheme and expected species
displayed on the cell membrane

| | Expected species displayed | | |
|---|---|---|---|
| | on cell membrane | | |
| Transfected vectors | Fc | Kappa Lc | scFv-Fc |
| pHC + pScFv-Fc + pLC | − | − | − |
| pHC-M1M2 + pScFv-Fc-M1M2 + pLC | + | + | + |
| pHC-M1M2 + pScFv-Fc + pLC | + | + | + |
| pHC + pScFv-Fc-M1M2 + pLC | + | + | + |
| pScFv-Fc-M1M1 | + | − | + |
| pHC-M1M2 + pLC | + | + | − |

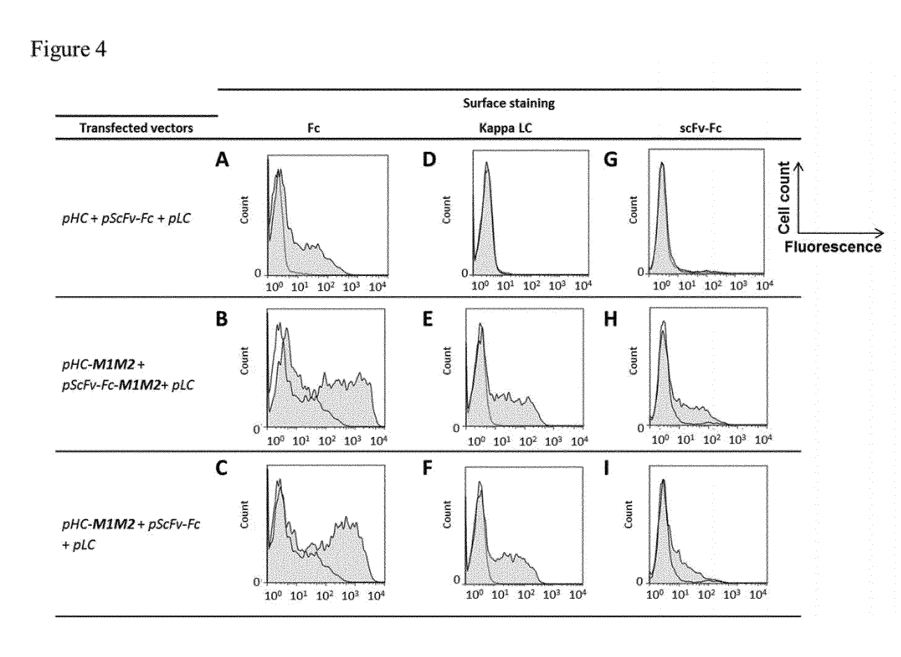

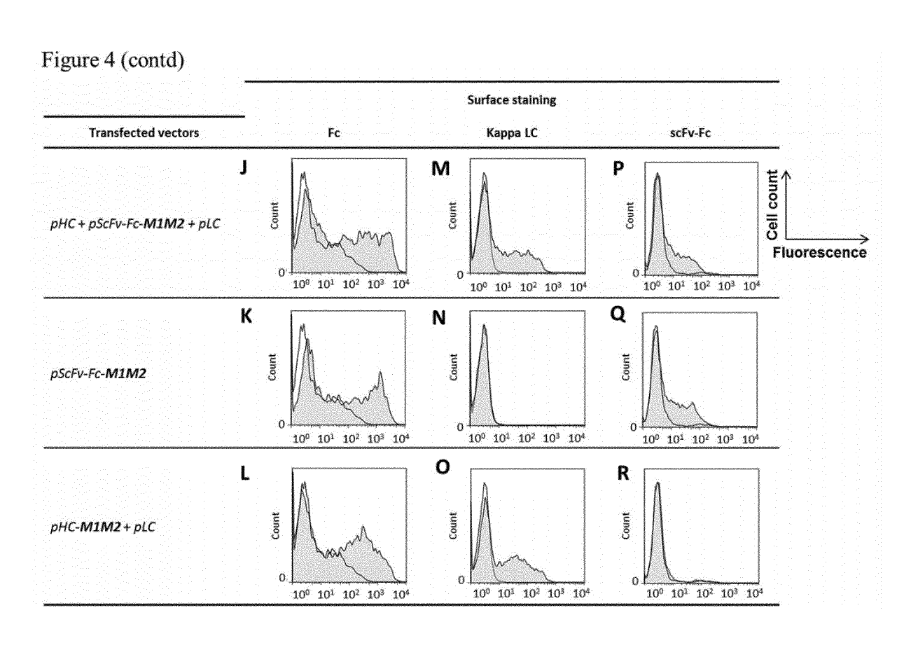

The flow cytometric profiles obtained on day 1 after transfection are shown in FIGS. 4A and B. The first transfection cocktail (pHC+pScFv-Fc+pLC) did not include a construct with a transmembrane region for cell membrane display. Thus, the profiles obtained for this transfection (FIG. 4A, D, G) were considered a negative control for the experiment and are presented as a dashed distribution in the histograms.

The transfection of the vector pScFv-Fc-M1M2 alone gave only positive cells for anti-Fc (detecting the heavy chain) and Protein A staining (FIG. 4K, N, Q). In this case, no light chain was detected on the cell membrane demonstrating the specificity of the assay. Co-transfection of vectors pHC-M1M2 and pLC yielded positive cells for anti-Fc and kappa light chain staining, but no Protein A binding could be observed (FIG. 4L, 0, R). This was in accordance with the fact that the Protein A binding site had been abrogated in the heavy chain subunit of the BEAT molecule. Hence, although the anti-human Fc gamma antibody was not specific for the heavy chain and recognized the scFv-Fc as well, the detection of light chain allowed us to conclude on the expression of the heavy chain.

Transfections performed with plasmid cocktails coding for all three subunits, independent of the alternate splicing construct used, showed expression of the BEAT construct with at least one transmembrane region (FIG. 4B, C, J, E, F, M, H, I, P). The staining was positive for the light chain and the scFv-Fc, as well as for the Fc fragments (heavy chain and scFv-Fc), indicating that all expected species were present on the cell membrane. Hence, fully folded multimers were present on the cell membrane confirming the effectiveness of the reporter system for the expression of multimeric proteins. Surprisingly, all species were detected regardless of the position of the transmembrane region. No difference was observed whether the transmembrane region (TM) was attached to both the heavy chain (HC) and the scFv-Fc subunit (FIG. 4B, E, H), solely to the HC (FIG. 4C, F, I) or solely to the scFv-Fc (FIG. 4J, M, P).

Therefore, the approach presented here allows the distinction between a heterodimer (for instance heavy chain+scFv-Fc+light chain) and homodimers (heavy chain+light chain or scFv-Fc homodimer) displayed on the cell membrane. In the context of expression of multimeric proteins such as bispecific antibodies this quality of information is tremendously useful.

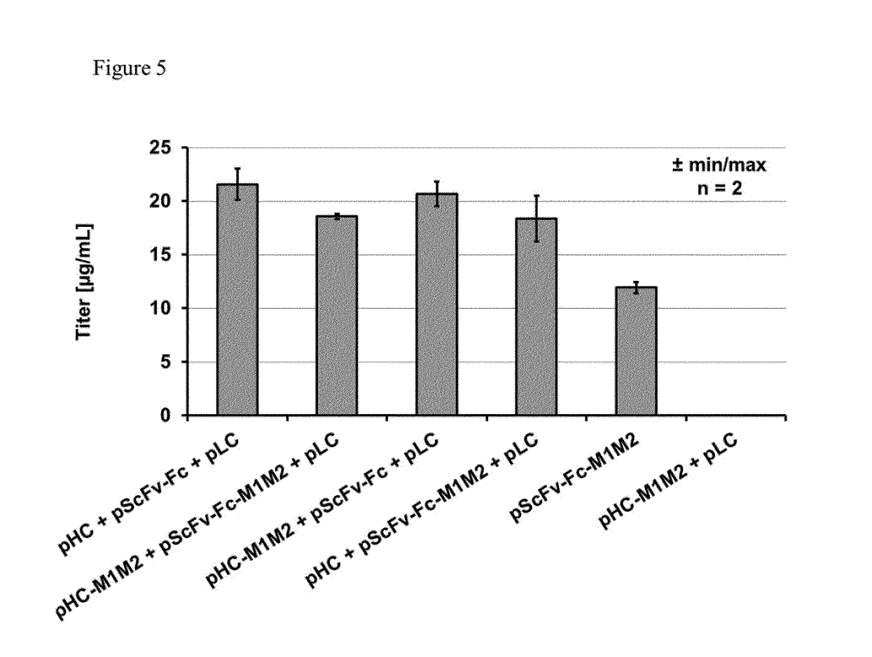

The concentrations of the secreted BEAT and scFv-Fc homodimer were measured on day 6 after transfection by Octet QK instrument using Protein A biosensors. The results are shown in FIG. 5. As expected no expression could be measured with the Protein A bio sensors for the transfection using the expression vectors pHC-M1M2+pLC as the resulting product (a heavy chain-light chain homodimer) does not bind to Protein A. For the transfection using the expression vector pScFv-Fc-M1M2 a lower expression level was observed compared to the control transfection leading exclusively to secreted product. BEAT molecules or scFv-Fc homodimer could be detected in the supernatants for all transfections using alternate splicing constructs. This confirms that the BEAT heterotrimers were not only displayed on the cell membrane as previously demonstrated, but were also successfully secreted using the secretory pathway of the cells. More importantly, the expression levels obtained with the alternate splicing construct were comparable to the control (pHC+pScFv-Fc+pLC). This suggested that only a small fraction of proteins deviated from the secretory pathway for surface display, as no significant impact on the secretion level could be measured in this experimental set-up.

### Summary

Based on these results it can be concluded that the alternate splicing constructs were successfully deflecting a fraction of the produced antibody to the cellular membrane by alternate splicing. While the surface staining was low for the constructs containing the PTCRA transmembrane domain, the staining was high for the cells transfected with the constructs containing the M1M2 transmembrane region and even higher for the constructs containing the B7-1 transmembrane region. Besides the signal strength, the percentage of the cellular population that could be stained was surprisingly high in the transfections with the B7-1 constructs, considering that the M1M2 transmembrane region was selected for efficient membrane display of antibodies during the evolution of B-cells and should therefore represent the most efficient construct for membrane display of immunoglobulins.

The overall antibody expression level decreased when a portion of the secreted antibody was redirected to the cell membrane, but around 80% of the expression level of the non-spliced control was reached with the different constructs.

Molecules with more than two subunits could also be successfully displayed on the cell membrane using the alternate splicing constructs of the present invention, as demonstrated by the example of the BEAT® constructs. These constructs fused a transmembrane region to a small fraction of the expressed protein. Using a bispecific antibody (BEAT) as an example, no significant differences in the secretion level could be observed compared to the control transfection leading exclusively to secreted protein. The observation that there was no, or only a minor impact of the alternate splicing on the expression level, makes the technology acceptable for industrial applications. Moreover, it could be shown that the approach as described herein allowed specific detection of the multimers displayed on the cell membrane. Surprisingly, the transmembrane region could be added to the heavy chain or to the scFv-Fc or to both subunits, without an impact on the surface display or the expression level of the secreted protein.

Since the multimeric molecules displayed on the cell membrane by alternate splicing reflect the product composition of secreted multimeric proteins of a specific cell, this technology would allow a cytometry based qualitative prediction of the secretion profile on the single cell level. This is demonstrated in the Example 5.

### Example 3: Modulation of Cell Surface Display and Expression Titer by Modification of Intron Sequence

### Introduction

As demonstrated in Example 2, alternate splicing of a transmembrane region allows redirecting a portion of an otherwise normally secreted antibody to the cell surface. Nevertheless, this modification of the secretion process might have a negative impact on the secretion level of antibody. Fine-tuning the splicing ratio between secreted and membrane displayed antibody might help recovering the expression level observed with the non-spliced antibody constructs (without an alternate splicing transmembrane region). This will be demonstrated in the following example.

### Material and Methods

### Transfections

Suspension CHO-S cells were transfected with expression vectors using polyethyleneimine (JetPEI®, Polyplus-transfection, Illkirch, France) in 50 ml bioreactor tube (Tubespins, TPP) format. For this purpose, exponential growing cells were seeded at a density of 2 E6 cells/mL in 5 mL of OptiMEM medium (#31985-047, Invitrogen). A JetPEI®:DNA complex was added to the cells in a weight ratio of 3 (μg/μg). Final DNA concentration in the cell suspension was 2.5 μg/mL. After 5 hours incubation at 37° C. under shaking (200 rpm), 5 mL of fresh culture medium were added to the cell suspension. Then the cells were incubated on a shaken platform at 37° C., 5% CO₂ and 80% humidity.

### Surface Staining

Surface staining of the cells was performed on day 1 after transfection. A total of 1 E5 cells were harvested and transferred to a round bottom well of a 96 well plate. The cells were washed twice with washing buffer (2% FBS in PBS) and then resuspended in 100 μL washing buffer containing the detection antibody. The specific detection of the heavy chain was performed using a PE conjugated goat anti human IgG antibody (#512-4998-82, eBioscience) excited by a blue laser (488 nm) and detected in the spectral range 583/26 nm. After 20 min incubation in the dark at room temperature, the cells were washed once in washing buffer and resuspended in 200 μL of washing buffer for flow cytometric analysis. The cells were analysed with a Guava flow cytometer (Merck Millipore) or with a FACSCalibur (Becton Dickinson). Transient expression level of secreted molecules was measured on day 4-6 after transfection using the Octet QK instrument (Fortebio, Menlo Park, Calif.) and protein A biosensors.

### Results

CHO-S cells were transfected as described in the Material and Methods section, using the expression vector pGLEX41_LC (SEQ ID NO: 294) expressing the light chain and a second vector coding for the heavy chain and containing modified versions of the intron with the M1M2 transmembrane domain (SEQ ID NO: 36-39), with the PTCRA transmembrane domain (SEQ ID NO: 40-43), with the M1M2-PTCRA fusion transmembrane domain (SEQ ID NO: 44-47) or with the B7-1 transmembrane domain (SEQ ID NO: 53-56). As control, the secreted heavy chain without alternate splicing was used (SEQ ID NO: 48).

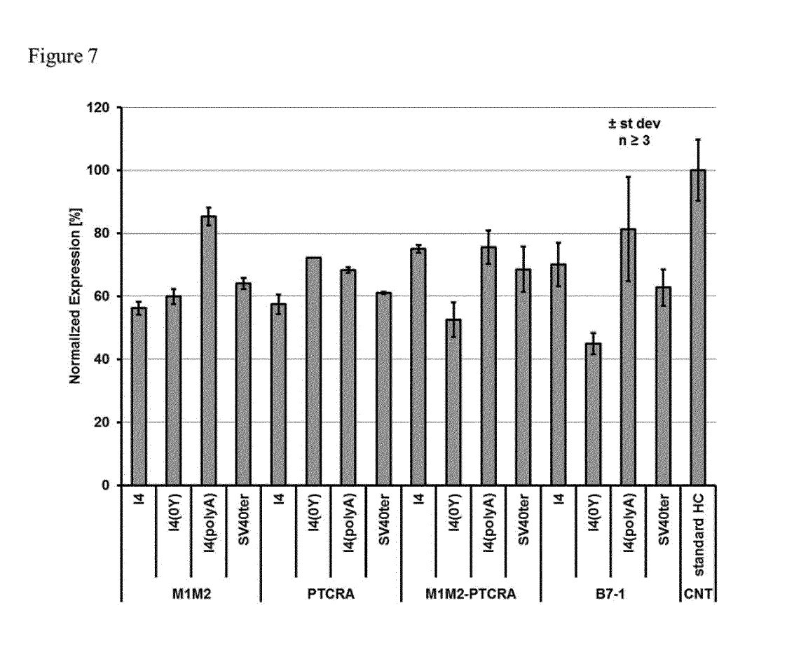

After transfection, the cells were stained as described in Materials and Methods and analyzed by flow cytometry. As already observed in Example 2, the negative control expressing the secreted version of light chain and heavy chain (without alternate splicing of a transmembrane region) only yielded a low fraction of stained cells (see FIG. 6) with a substantial level of IgG1 secretion (see FIG. 7). In contrast to this, cells transfected with constructs containing the M1M2 region (SEQ ID NO: 36, FIG. 6A) and the B7-1 transmembrane domain (SEQ ID NO: 55, FIG. 6M) showed a significant positive population displaying the antibody on the cell membrane. With the PTCRA transmembrane domain (SEQ ID NO: 40, FIG. 6E) or the M1M2-PTCRA fusion transmembrane domain (SEQ ID NO: 44, FIG. 61), the positive population was weakly stained.

The reduction of the Y content in the poly(Y) tract of the intron has been shown to weaken the splice acceptor and was introduced in order to shift the splice ratio in favor of secreted antibody. For both transmembrane regions leading to successful display (M1M2 and B7-1), the constructs without Y in the poly(Y) tract of the intron (SEQ ID NO: 37 and SEQ ID NO: 53) showed no surface staining of cells (see FIGS. 6B and N). This suggested that the ratio in splicing was mostly shifted towards the secreted antibody.

The presence of an additional poly(A) site in the intron of the mouse immunoglobulin μ primary transcript has been shown to impact the alternate splicing event, leading to a higher fraction of secreted product (Galli et al., 1987) and less membrane displayed antibody. Based on this finding, an SV40 poly(A) site was introduced 3′ of the stop codon of the secreted polypeptide and 5′ of the branch point of the intron in order to shift the splice ratio in favor of antibody secretion. With the addition of the SV 40 poly(A) site in the intron the secretion level of antibody was significantly increased for the M1M2 construct compared to the original 14 one (see FIG. 7). At the same time, the fraction of membrane displayed antibody for the M1M2 construct (see FIG. 6C) was increased. As the staining of the B7-1 cells was already very high, no conclusions could be drawn on the impact of the poly(A) on the membrane display (see FIG. 60).

The gastrin terminator site (reported to terminate mRNA transcription) was introduced directly 3′ of the poly(A) site of the expression cassette in order to avoid aberrant splice events with other splice acceptors in proximity of the expression cassette. The gastrin terminator construct increased the fraction of membrane displayed antibody especially for the M1M2 construct (FIG. 6D), but did not lead to a significant increase in the secretion level. Again, no conclusion could be drawn for the B7-1 cells (FIG. 6P).

Alternate splicing and hence, the membrane-bound expression of a portion of the overall produced antibody seems to have a negative impact on the overall secreted IgG titers. Nevertheless, the M1M2 transmembrane region (also in combination with the PTCRA transmembrane domain) and especially the B7-1 transmembrane region allowed up to 80% of the secretion level of the control construct without alternate splicing (see FIG. 7). Furthermore, the addition of a poly(A) signal in the intron was found to be favorable for IgG secretion, whilst maintaining a significant staining on the cell membrane. The gastrin terminator on the other hand showed only a minor impact on secretion compared to the respective reference constructs (see FIG. 7).

In the previous experiments, the different modifications of the intron sequence showed a similar impact on the efficiency of alternate splicing, independent of the transmembrane region of the construct. Therefore the B7-1 transmembrane region was chosen to further characterize the impact of intron sequence modifications on the alternate splice ratio and the antibody secretion level, as it was leading to the highest levels of staining.

In order to reduce the amount of cell surface displayed antibody and to restore the expression level of the control construct, the splice ratio was altered in favor of the secreted product, i.e. the splice donor and the splice acceptor site were weakened.

The poly(Y) tract present in the splice acceptor consensus sequence is known to play an important role in the splice acceptor strength (the shorter the poly(Y) tract, the weaker the splice acceptor site). In the precedent experiment, it was shown that complete reduction of the Y content in the poly(Y) tract to zero abolishes the cell surface display of the antibody.

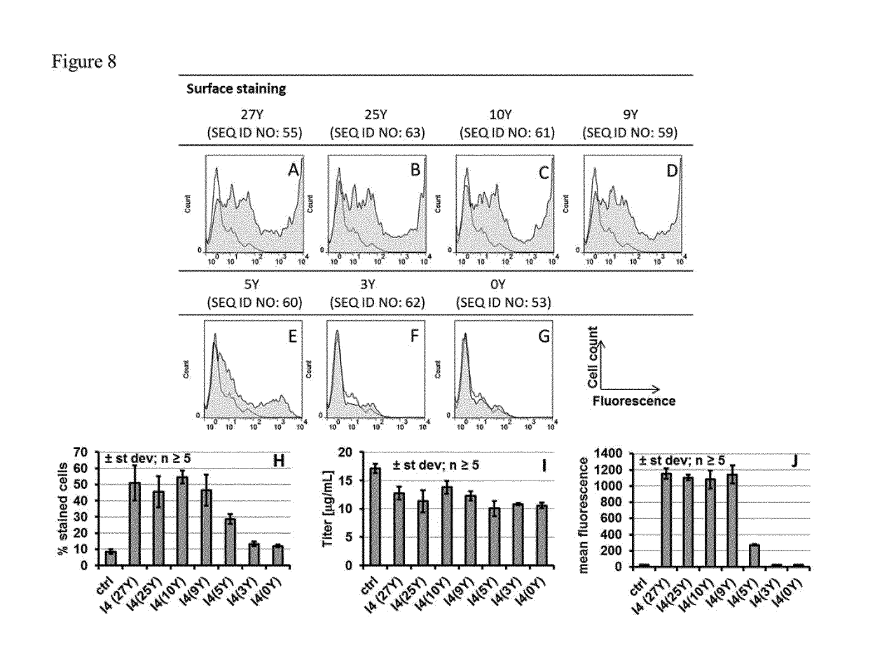

The native chicken TNT intron 4 poly(Y) tract contains 27 Y. A series of constructs with modified introns containing reduced numbers of Y in their poly(Y) tract were transfected in CHO-S cells as described previously (SEQ ID NO: 53, 55, 59-63). After surface staining and estimation of the expression level using the Octet device and protein A biosensors, an impact of the poly(Y) tract could be clearly identified on the cell surface display of the antibody: less than 9Y in the poly(Y) tract reduced dramatically the percentage of stained cells (see FIG. 8 A, B, C, D, E, F, G, H) as well as the staining intensity (see FIG. 8 A, B, C, D, E, F, G, J). Unfortunately, this reduction in cell surface display had no impact on the overall expression level which remained lower than the control for every construct (see FIG. 8 I).

Another way to increase the splicing ratio towards the secreted product is to reduce the strength of the splice donor at the 5′ end of the alternatively spliced intron by changing the DNA sequence of the splice donor. Two different DNA constructs (SD_CCC, SEQ ID NO 58 and SD_GGC, SEQ ID NO 57) coding for same amino acid sequence as the original construct could be generated by taking advantage of alternate codons coding for the same amino acid.

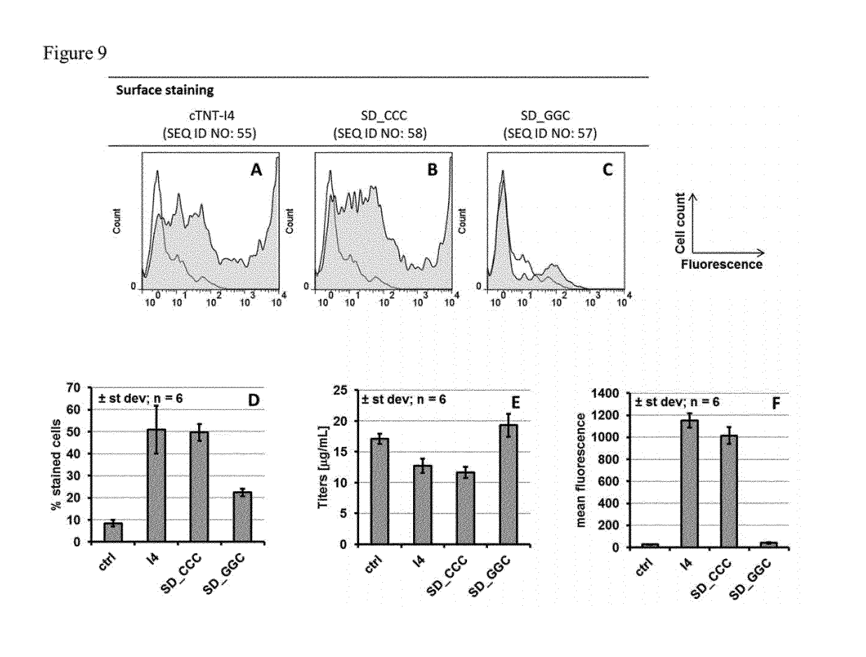

While the first modification of the splice donor consensus sequence (SD_CCC, SEQ ID NO 58) had no effect on either percentage of stained cells, intensity of staining or expression level (see FIG. 9), the second one (SD_GGC, SEQ ID NO 57) showed a dramatic decrease in surface display (both percentage and intensity, see FIGS. 9 C, D and F). This decrease in surface display correlated with a clear increase in expression titer, up to the level of the control without alternate transmembrane domain (see FIG. 9E). Furthermore, although the cell surface display was dramatically decreased when compared to the native chicken TNT intron 4 sequence, a significant staining could be observed when compared with the control without transmembrane domain (see FIG. 9C).

### Summary

The gastrin terminator increased the fraction of cells stained positive for IgG on the cell membrane especially for the M1M2 construct, but did not lead to higher expression of secreted IgG compared to the standard construct. The insertion of an additional poly(A) in the intron increased the fraction of membrane displayed antibody in the constructs M1M2 (for B7-1 no conclusion could be drawn). The M1M2 construct with the additional poly(A) also showed high levels of secreted IgG (up to 80% of the expression of the non-spliced control constructs).

Reduction of the poly(Y) tract was expected to weaken the splice acceptor and to reduce the expression of the membrane displayed splice variant. This could be confirmed for all four different basal constructs (M1M2, PTCRA, M1M2-PTCRA and B7-1). Furthermore, the level of surface display was found to be directly correlated to the length of the poly(Y) tract, as depicted by the examples with the B7-1 transmembrane domain. A minimum of 5 Y in the poly(Y) tract was found necessary for a significant surface staining in this context. At the same time, no increase in the expression levels of secreted antibody could be observed. Hence, the shift of the alternate splicing did not benefit the accumulation of secreted product. Unexpectedly, a specific modification of the splice donor consensus sequence allowed decreasing the level of surface staining while increasing the level of secreted antibody. The level of surface display was quite low, but still significant as compared to the control construct, and the secretion level was found to be the same as the control.

The inventors consider that the frequency of the alternate splice event is determined by the strength of the splice donor site. If the corresponding splice acceptor is strong enough (more than 5 Y in the poly(Y) tract), alternate splicing will lead to the formation of the alternate mRNA coding for the construct with transmembrane region and hence membrane display. If the splice acceptor is weakened by lowering the amount of Y in the poly(Y) tract to less than 5 Y, the splicing event will be less efficient and the alternate pre-mRNA might be degraded, while there is no impact on the non-spliced mRNA and thus on the secretion level. In accordance with this, less membrane display was observed after weakening of the splice acceptor, but there was no impact on the secretion level. Reducing the strength of the splice donor on the other hand might reduce the frequency of the alternate splicing event. As a consequence, more mRNA is coding for the secreted product of the non-spliced mRNA, leading to the observed increase in secreted antibody. The strong splice acceptor leads to efficient alternate splicing of mRNA, but at a lower frequency due to the weak splice donor, which might explain the significant, but weak membrane display observed.

In summary, modifications of the splice donor and/or splice acceptor sequences allowed a modulation of both the level of surface display and the secretion level of the antibody up to the secretion level of an antibody expressed without alternate splicing. The presented constructs therefore allow the fine-tuning of the display and secretion up to the desired levels by adjusting the alternate splicing efficiency.

Galli G1, Guise J, Tucker P W, Nevins J R (1988). Poly(A) site choice rather than splice site choice governs the regulated production of IgM heavy-chain RNAs. Proc Natl Acad Sci USA. April; 85(8):2439-43.

### Example 4: Impact of Transmembrane Region on Cell Surface Display

### Introduction

The alternate splicing system for the membrane display of a fraction of antibody produced by cells might be altered by characteristics of the transmembrane region. For example, it could be speculated that the length and the structure of the transmembrane domain may influence the efficacy of the membrane display. Furthermore, the cytosolic tail of the transmembrane region might impact the cell surface display by presence or absence of an ER exportation signal. In the following example, we show that neither the amino acid composition of the transmembrane domain, its length, nor the cytosolic tail of the transmembrane region have a critical impact on surface display and secretion.

### Material and Methods

### Transfections

Suspension CHO-S cells were transfected with the expression vectors using polyethyleneimine (JetPEI®, Polyplus-transfection, Illkirch, France) in 50 ml bioreactor tube (Tubespins, TPP) format. For this purpose, exponential growing cells were seeded at a density of 2 E6 cells/mL in 5 mL of OptiMEM medium (#31985-047, Invitrogen). A JetPEI®:DNA complex was added to the cells in a weight ratio of 3 (μg/μg). Final DNA concentration in the cell suspension was 2.5 μg/mL. After 5 hours incubation at 37° C. under shaking (200 rpm), 5 mL of fresh culture medium were added to the cell suspension. Then the cells were incubated on a shaken platform at 37° C., 5% CO₂ and 80% humidity.

### Surface Staining

Surface staining of the cells was performed on day 1 after transfection. A total of 1 E5 cells were harvested and transferred to a round bottom well of a 96 well plate. The cells were washed twice with washing buffer (2% FBS in PBS) and then resuspended in 100 μL washing buffer containing the detection antibody. The specific detection of the heavy chain was performed using a PE conjugated goat anti human IgG antibody (#512-4998-82, eBioscience) excited by a blue laser (488 nm) and detected in the spectral range 583/26 nm. After 20 min incubation in the dark at room temperature, the cells were washed once in washing buffer and resuspended in 200 μL of washing buffer for flow cytometric analysis. The cells were analysed with a Guava flow cytometer (Merck Millipore) or with a FACSCalibur (Becton Dickinson). Transient expression level of secreted molecules was measured on day 4-6 after transfection using the Octet QK instrument (Fortebio, Menlo Park, Calif.) and protein A biosensors.

### Results

CHO-S cells were transfected as described in the Material and Methods section, using the expression vector pGLEX41_LC (SEQ ID NO: 294) expressing the light chain and a second vector coding for the heavy chain with different transmembrane regions. As control, the secreted heavy chain without alternate splicing was used (SEQ ID NO: 48) and the construct with the B7-1 transmembrane region (SEQ ID NO: 55) was used as positive control.

After transfection, the cells were stained as described in Materials and Methods and analyzed by flow cytometry.

### Transmembrane Domain Length and Composition

In order to analyze the impact of the transmembrane domain on cell surface display and antibody secretion levels, the B7-1 transmembrane domain was replaced by transmembrane domains of similar length (22-23 amino acids) containing only hydrophobic residues (see SEQ ID NO: 81-86). Exchanging the transmembrane domain had no impact on cell surface display or secretion of the antibody (see FIG. 10).

When the B7-1 transmembrane domain was replaced by transmembrane domains of similar length (21-24 amino acids) containing not exclusively hydrophobic residues, but also polar or charged residues (SEQ ID NO: 87-93) no impact could be seen on surface display or on secretion, except for the transmembrane domain derived from PTCRA (SEQ ID NO: 93). This transmembrane domain, without changing the fraction of cells exhibiting a product on the cell surface, reduced dramatically the surface density of the product without any impact on secretion level (see FIGS. 11 H, J, K and L)

Transmembrane domains are surrounded by a hydrophobic lipid phase. The energy cost of inserting an ionizable group in the hydrophobic environment of the membrane is very high, therefore there should be a strong bias against charged amino acids in the transmembrane domain (White and Wimley, 1999). A statistical analysis of transmembrane helices confirmed that transmembrane domains are composed mainly by hydrophobic amino acid residues, particularly in the hydrophobic core of the transmembrane region (Beza-Delgado, 2012). Based on this observation we wanted to identify the impact of three charged residues (R8, K13 and D18, the numbering starting at the beginning of the transmembrane helix) in the PTCRA transmembrane domain on the observed weak staining intensity. Several PTCRA constructs were designed where different charged residues were exchanged for the amino acid valine (one of the four most frequent amino acids in the transmembrane domain (Baeza-Delgado et al., 2012)) and transfected in CHO-S cells (SEQ ID NO: 94-100). The results of these transfections confirmed the impact of the charged residues on the surface staining intensity. Mutation of the single amino acid R8V (SEQ ID NO: 94), K13V (SEQ ID NO: 95) or D18V (SEQ ID NO: 96) allowed to increase significantly the density of surface displayed antibody, but had no significant impact on the secretion level of the antibody (see FIGS. 12 B, C, D and J). As expected from the results of the single mutations, the combinations of 2 mutated charged residues (either R8V and K13V (SEQ ID NO: 97), R8V and D18V (SEQ ID NO: 98) or K13V and D18V (SEQ ID NO: 99)) showed a level of surface display and a similar secretion level than the transmembrane domain of PTCRA (see FIGS. 12 E, F, G and J). Finally, the mutation of all three charged residues to valine (SEQ ID NO: 100) resulted as expected in a high surface display compared to the control. Unexpectedly, the expression level of secreted antibody, as well as the percentage of stained cells was slightly lower with this construct (see FIGS. 12 H and J). The reason for the reduced secretion and staining levels is currently unclear.

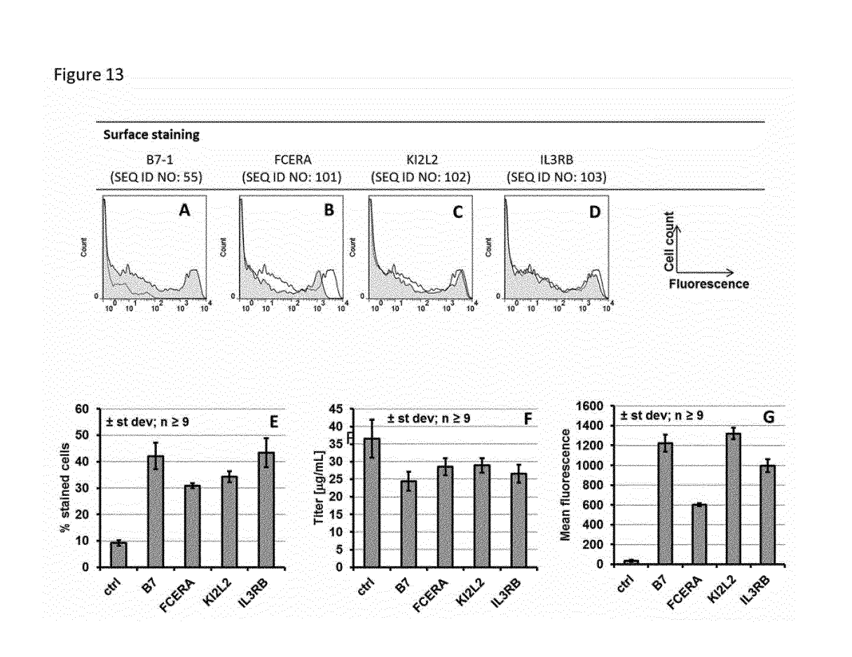

When the B7-1 transmembrane domain was replaced by shorter transmembrane domains (17-19 amino acids) containing not only hydrophobic residues, but also polar or charged residues (SEQ ID NO: 101-103), different effects could be identified. The results can be seen in FIG. 13. Whereas the transmembrane domain of FCERA (SED ID NO: 101) and KI2L2 (SEQ ID NO: 102) had a minor detrimental effect on the percentage of stained cells (FIGS. 13 B, C and E), only the transmembrane domain derived from FCERA had a negative impact on the intensity of the staining (FIGS. 13 C and G). Furthermore, both transmembrane domains (FCERA and KI2L2) had a minor favorable effect on the secretion level (FIG. 13F). For the transmembrane domain from IL3RB (SEQ ID NO: 103) on the other hand, no change compared to the B7-1 reference construct was seen for the percentage of stained cells and the secretion level (FIGS. 13 D, E and F), only a slight decrease in staining intensity was observed (FIG. 13 G). This makes the short transmembrane domains a potential tool for fine-tuning of the product surface display.

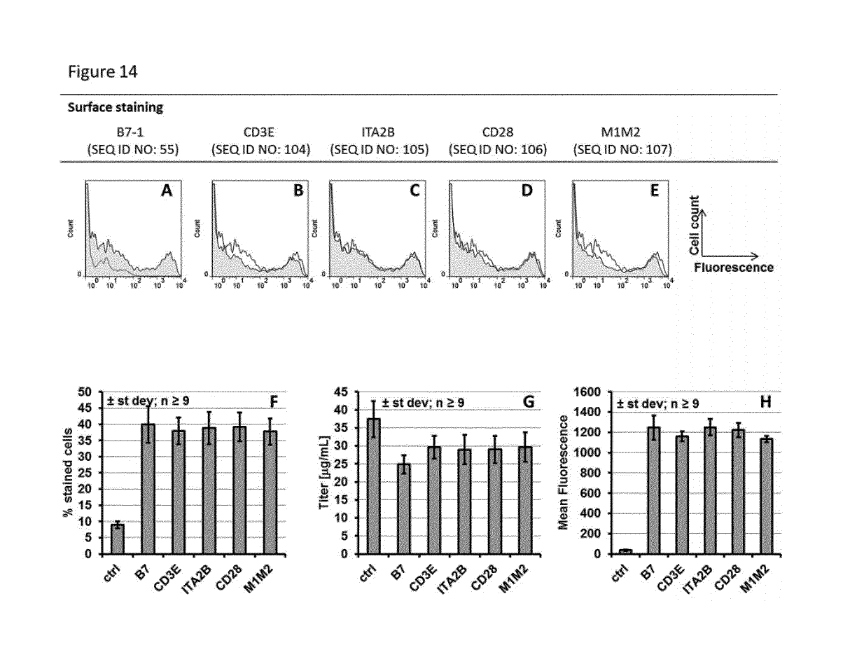

When the B7-1 transmembrane domain was replaced by longer transmembrane domains containing not only hydrophobic residues, but also polar or charged residues (SEQ ID NO: 104-107), no effect was observed on the percentage of stained cells, nor on the staining intensity compared to the reference construct with the B7-1 transmembrane domain (see FIGS. 14 B, C, D, E,F and H). Nevertheless the secretion was slightly better with all these transmembrane domains than with B7-1 (see FIG. 14 G).

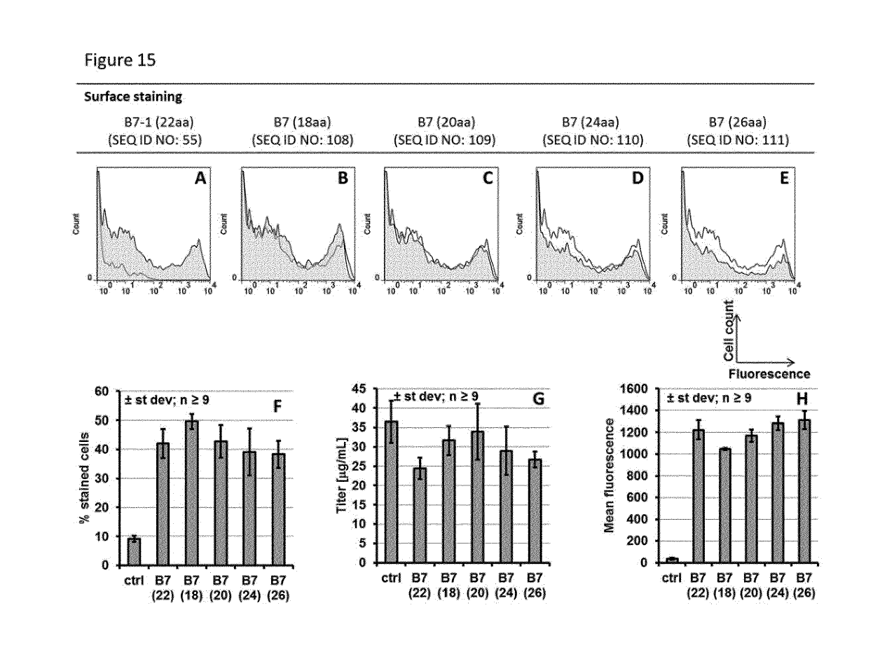

In order to assess the impact of the transmembrane domain length in a more defined way without switching from one transmembrane domain to another, constructs with modified B7-1 transmembrane domains were designed. Several hydrophobic amino acids were removed from or added to the hydrophobic center of the B7-1 transmembrane domain (SEQ ID NO: 108-111). Shortening B7-1 to 18 amino acids (SEQ ID NO: 108) was beneficial in terms of percentage of stained cells, but slightly detrimental in term of staining intensity (see FIGS. 15 B, F and H). The three other constructs (20 (SEQ ID NO: 109), 24 (SEQ ID NO: 110) and 26 (SEQ ID NO: 111) amino acids, respectively) did not significantly influence the percentage of stained cells or the staining intensity (see FIGS. 14 C, D, E, F and H). Regarding the secretion level, shortening the length of the transmembrane domain was found beneficial, whereas constructs with elongated transmembrane domain showed a comparable secretion level as constructs with the native B7-1 (see FIG. 14G).

In general, a shorter transmembrane domain seems to be favorable in terms of percentage of stained cells and in terms of secretion of antibody, although the staining intensity might be slightly decreased compared to longer transmembrane domains.

### Cytosolic Tail

Literature suggests the presence of a structural, non-linear ER exportation signal in the B7-1 cytosolic tail (Lin et al., 2013). The authors found that the presence of an ER exportation signal in the cytosolic domain of a transmembrane protein targeted to the cell surface might have a positive impact on the amount of protein on the cell surface. In the case of a protein with a high turn-over, the accelerated export from the ER to the membrane may compensate for proteolytic cleavage from the plasma membrane.

In order to assess whether the ER exportation signal is relevant for the membrane display in our system, the B7-1 cytosolic tail was replaced by the cytosolic tails of several transmembrane proteins known to contain an ER exportation signal and deletion mutants without the ER exportation signal (SEQ ID NO: 112-124).

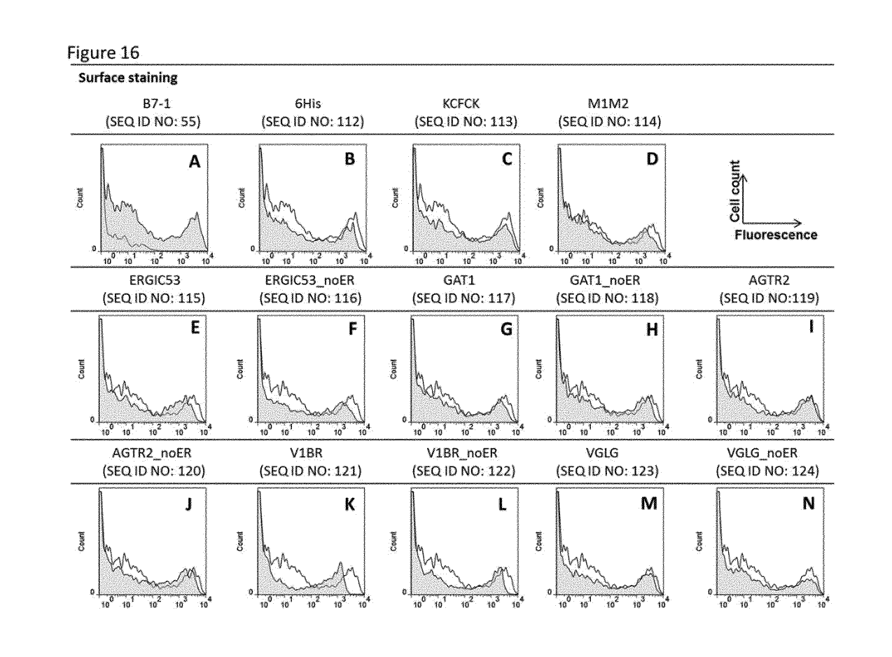

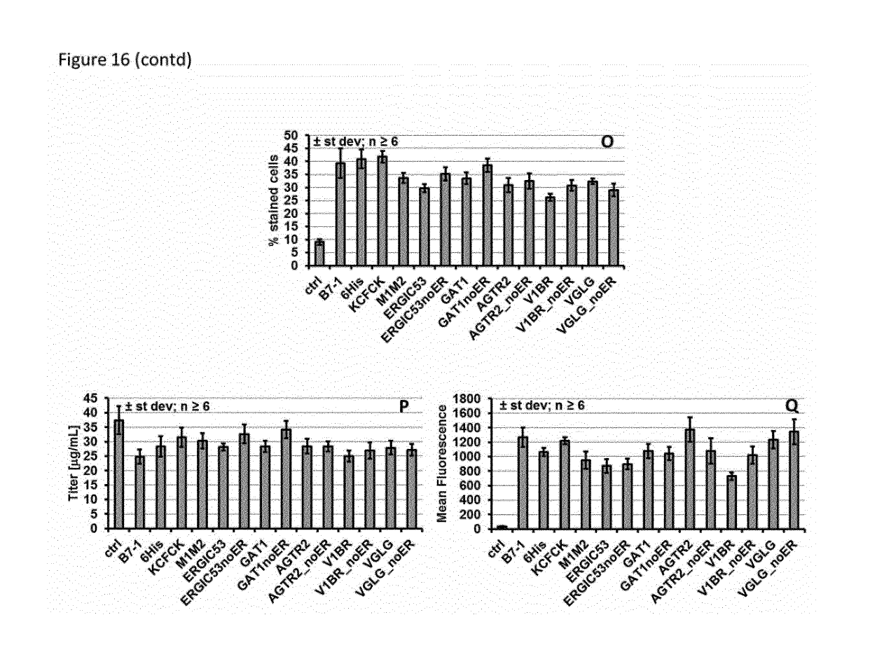

As shown in FIG. 16 P, the absence of the ER exportation signal was found slightly favorable for the level of secreted antibody, while the percentage of stained cells (FIGS. 16 B-N and O) and the staining intensity (FIGS. 16 B-N and Q) were not significantly affected. Hence, our membrane displayed antibody might not be subject to a rapid turnover.

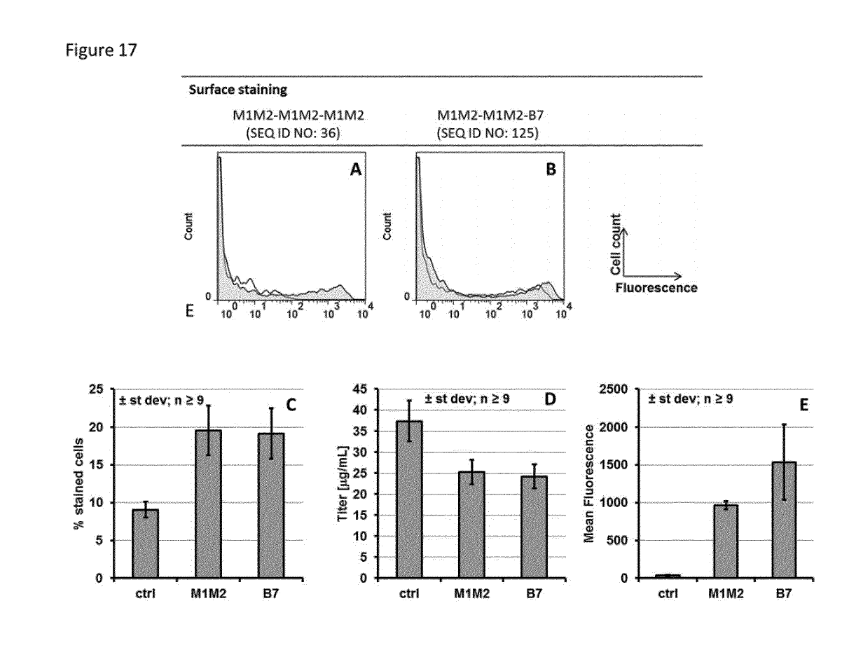

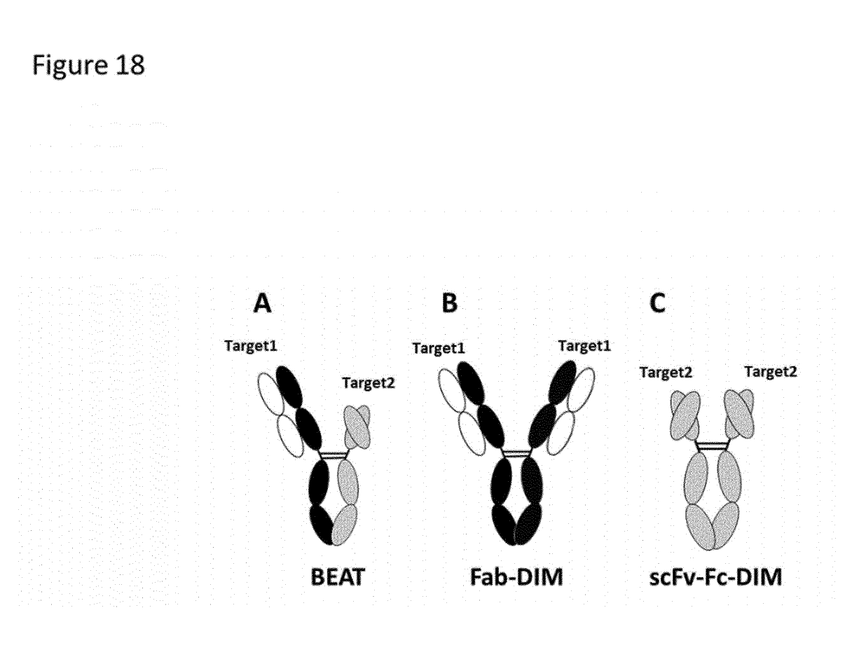

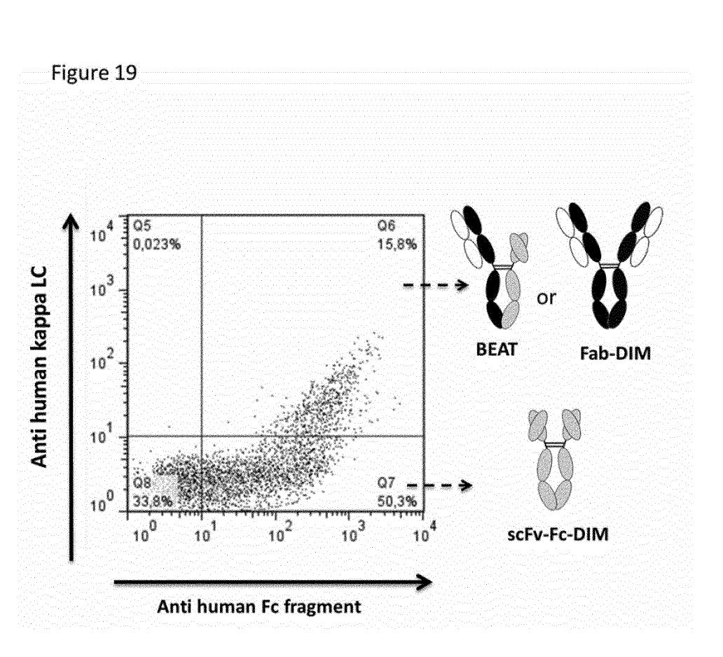

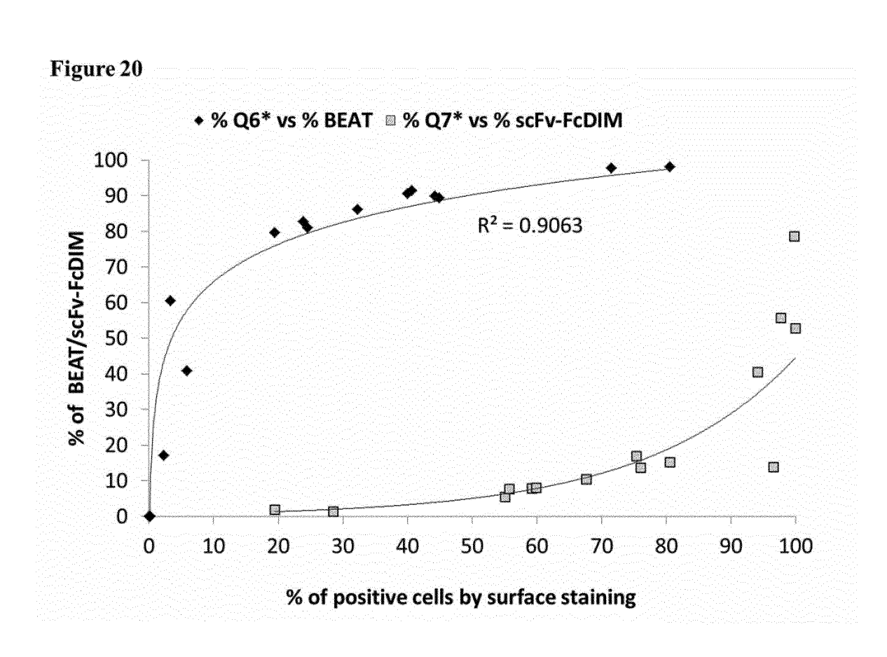

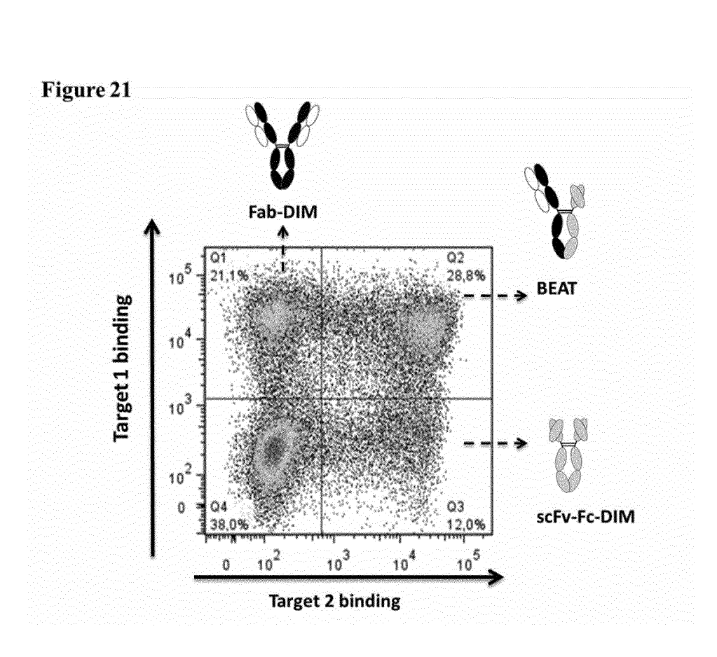

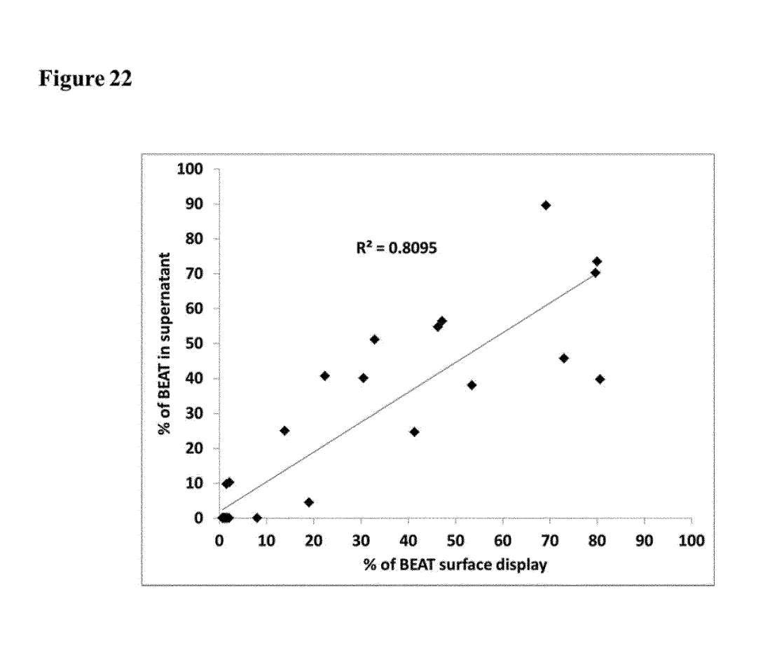

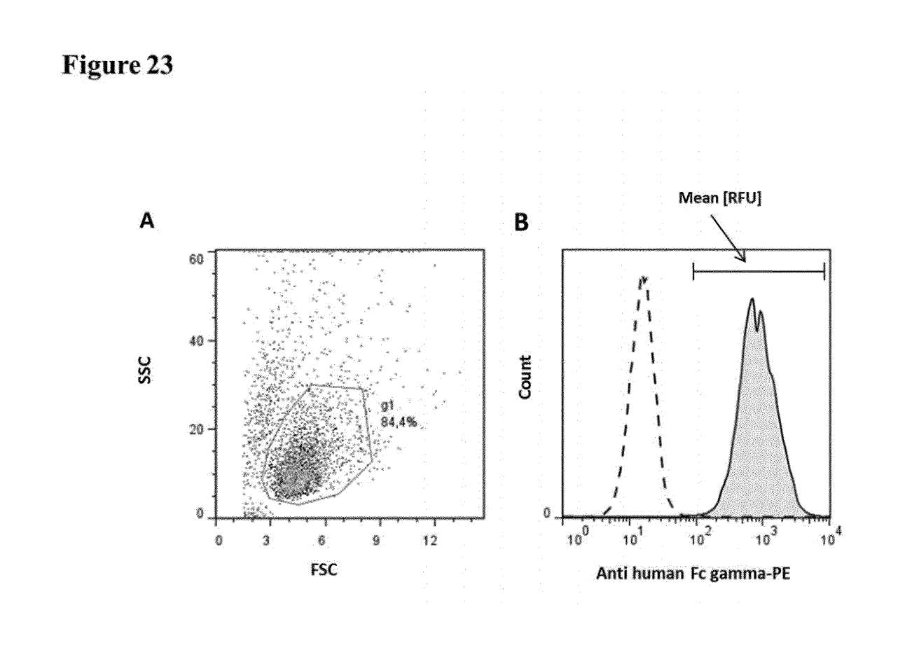

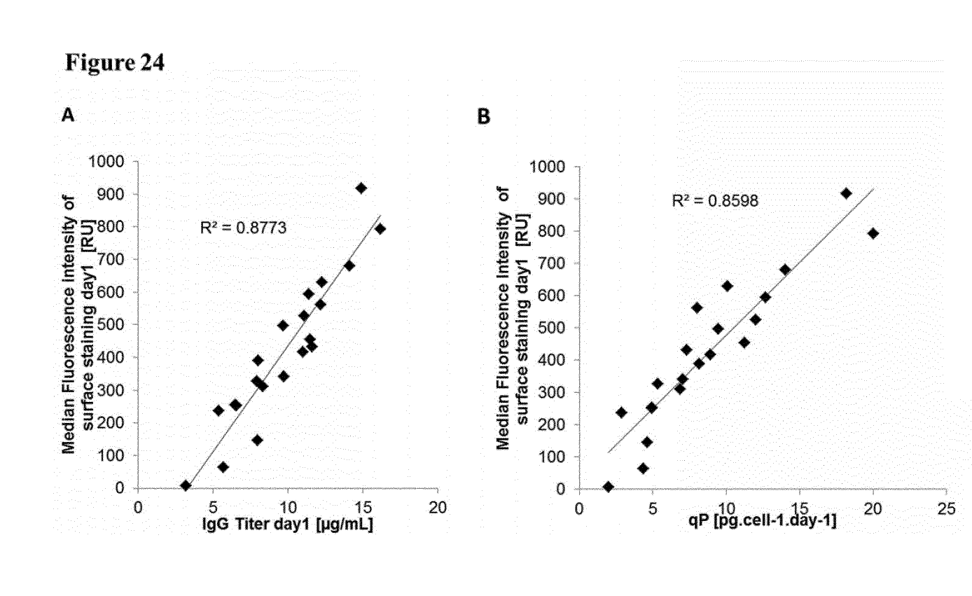

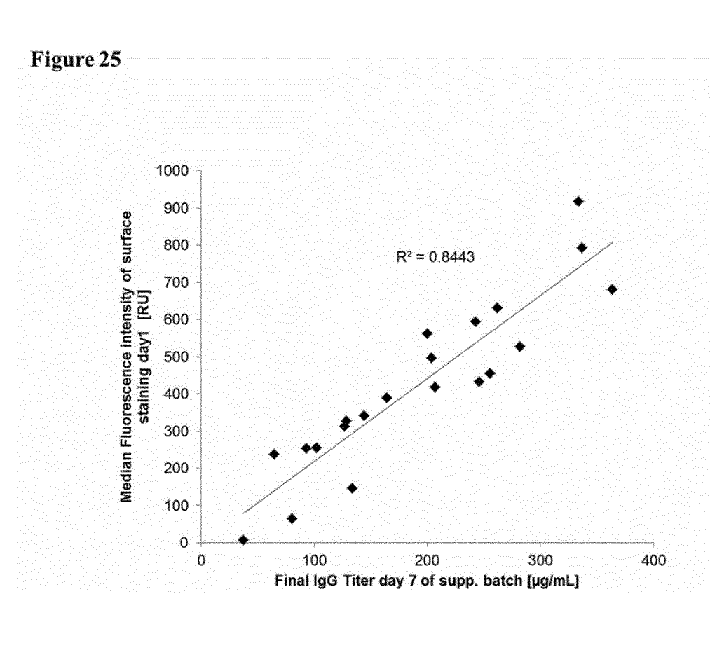

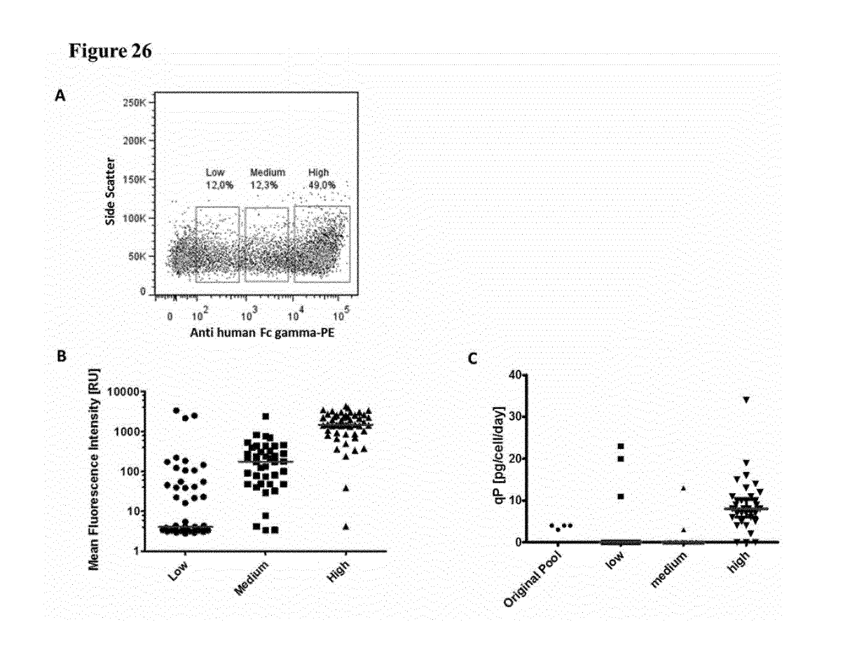

In addition to these constructs, the exchange of the M1M2 cytosolic tail by the B7-1 cytosolic tail in M1M2 transmembrane domain was performed (SEQ ID NO: 125). As shown in FIG. 17 E, this exchange increased slightly the intensity of the fluorescence, but had no impact on the percentage of stained cells (FIG. 17 C) nor on the secretion level (FIG. 17 D).

### Summary

Taken together, the data suggest that the length or the amino acid composition of transmembrane domains in our alternate splicing system had only a minor impact on the secretion level of antibodies and the product display on the cell membrane. Minor shifts in the ratio of secreted to membrane-displayed product were observed that might be used for fine-tuning of the alternate splicing system. All of the constructs allowed a significant surface display of the antibody and also a relevant secretion level.

Interestingly, a major impact of the transmembrane domain was seen on the fluorescence level of the cell surface display. Presence or absence of hydrophobic amino acids allowed to control the mean fluorescence, while it did not impact the secretion level or the percentage of cells showing cell surface display.

The ER exportation signal in the cytoplasmic tail was found to be not necessary for the cell surface display of the control antibody. A minimal cytoplasmic tail consisting of a glycine-alanine linker and 6 histidines or the first 5 amino acids of the B7-1 tail were sufficient for cell surface display. This might be different for other antibodies or proteins subject to a rapid turnover. Thus the cytosolic tail might be an interesting tool for adjusting the surface display of the protein of interest.

Taken together, the data from examples 2-4 suggests that surface display level and secretion level are influenced by the different sequences involved in the alternate splicing and the membrane integration of the membrane-bound protein. Whereas specific modification of the splice donor site allowed recovering the same secretion level than the control non-alternate splicing construct (see Example 3 FIG. 9), the surface staining was significantly decreased when using B7-1 transmembrane region. On the other hand, adding a SV40 poly(A) in the intron when using M1M2 transmembrane region increased both secretion and surface display levels (see Example 3 FIGS. 6 and 7). Moreover, modifications of the B7-1 transmembrane domains as B7-1 reduced to 18 or 20 amino acids (see FIG. 15) had a positive impact on both the secretion level and the surface display level. Finally, replacing the B7-1 cytosolic tail by a cytosolic tail containing no ER export signal as 6His or KCFCK also improved both surface display and secretion levels. Finally, the impact of Gastrin terminator at the end of the expression cassette on expression was positive, and might be even more favorable if placed in the intron. Therefore, constructs combining these different features might be even more favorable, allowing the best secretion level coupled with a high dynamic range of cell surface display wished for cell selection.

The backbone of these constructs will be the pGLEX41 (GSC281, SEQ ID NO: 304) and the sequences of the last 5 amino acids of the non-spliced ORF to the end of the expression construct are listed as DNA sequences as well as the protein sequence starting from the same amino acids with the fused transmembrane region as protein sequences in Fehler! Ungültiger Eigenverweis auf Textmarke.

TABLE 17
Sequences of the potential constructs (*)

| | DNA | Protein |
|---|---|---|
| | sequence | sequence |
| Construct name | SEQ ID NO: | SEQ ID NO: |
| pGLEX41_HC-GGC-I4(polyA)-B7-B7(18)- | 331 | 332 |
| 6His | | |
| pGLEX41_HC-GGC-I4(polyA)-B7-B7(20)- | 335 | 336 |
| 6His | | |
| pGLEX41_HC-GGC-I4(polyA)-B7-B7(18)- | 333 | 334 |
| KCFCK | | |
| pGLEX41_HC-GGC-I4(polyA)-B7-B7(20)- | 337 | 338 |
| KCFCK | | |
| pGLEX41_HC-GGC-I4(SV40ter)-B7- | 329 | 330 |
| B7(18)-6His | | |
| pGLEX41_HC-GGC-I4(SV40ter)-B7- | 341 | 342 |
| B7(20)-6His | | |
| pGLEX41_HC-GGC-I4(SV40ter)-B7- | 339 | 340 |
| B7(18)-KCFCK | | |
| pGLEX41_HC-GGC-I4(SV40ter)-B7- | 343 | 344 |
| B7(20)-KCFCK | | |

(*) Sequence listed stretches from the last 5 amino acids of the non-spliced ORF to the end of the expression construct

### PUBLICATIONS

Lin, Y., Chen, B., Wei-Cheng Lu, W., Chien-I Su, C., Prijovich, Z., Chung, W., Wu, P., Chen, K., Lee, I., Juan, T and Roffler, S. (2013). The B7-1 Cytoplasmic Tail Enhances Intracellular Transport and Mammalian Cell Surface Display of Chimeric Proteins in the Absence of a Linear E R Export Motif PLoS One. September 20; 8(9)
Baeza-Delgado, C1, Marti-Renom, M A, Mingarro, I. (2012). Structure-based statistical analysis of transmembrane helices. Eur Biophys J. 2013 March; 42(2-3):199-207
White, S H and Wimley, W C. (1999). Membrane protein folding and stability: physical principles. Annu Rev Biophys Biomol Struct. 28:319-65.

### Example 5: Qualitative Prediction of Product Secretion Using Alternate Splicing for Surface Display

### Introduction

Transfection of cells is the initial step for the establishment of a stable cell line for recombinant protein expression. For many reasons (heterogeneous initial cell population, different integration loci of plasmids in the genome, different post-translational machinery, and epigenetic regulation of expression) this step generally gives rise to heterogeneous cell populations with regard to expression and secretion level but also product quality attributes such as protein folding, glycosylation and other post-translational modification. Bispecific antibodies are composed by up to four different subunits (2 heavy chains and 2 light chains) that might assemble in all possible combinations. Depending on plasmid integration, regulation of transcription and translation as well as the folding efficacy in the ER, a particular clone may secrete preferably the correctly assembled product instead of unwanted by-products. The selection of a clone with this desired secretion pattern requires extensive screening and characterization of secreted proteins. Reducing the amount of screening needed in order to obtain a cell line producing the product of interest and minimal by-products, would be a major development.

It was demonstrated previously in Examples 2 to 4 that a fraction of secreted protein can be deviated from the secretory pathway for cell membrane display using alternate splicing technology. It was also shown that specific detection of proteins displayed on the cell membrane is possible via flow cytometry.

The aim of this example was to demonstrate that the product pattern display on the cell membrane is predictive of the secretion profile of a clone. For this purpose, the heterotrimeric bispecific BEAT® antibody presented in Example 2 was used as a model. The molecule binds to two different soluble molecules called “Target1” and “Target2” in the following. A schematic cartoon of the molecule can be seen in FIG. 18, structure A. It will be abbreviated “BEAT” in the following text. After transfection not only BEAT molecules are secreted but also the monospecific heavy chain-light chain dimer binding to “Target1”. This molecule is referred to as Fab homodimer, (abbreviated “Fab-DIM”). A second by-product is the monospecific scFv-Fc homodimer (abbreviated “scFv-Fc-DIM”) binding to “Target2”. These by-products can be seen as structures B and C in FIG. 18.

After stable transfection of cells with the expression vectors pGLEX41_BEAT-HC-I4-M1M2-M1M2-M1M2 (SEQ ID NO: 50), pGLEX41_scFv-Fc-I4-M1M2-M1M2-M1M2 (SEQ ID NO: 52) and pGLEX41_BEAT-LC (SEQ ID NO: 302) or with pGLEX41_BEAT-LC (SEQ ID NO: 302), pGLEX41_scFv-Fc-I4-B7-B7-B7 (SEQ ID NO: 323) and pGLEX41_BEAT-HC-I4-B7-B7-B7 (SEQ ID NO: 321), clones were screened by surface staining. Two approaches were followed for characterizing the surface phenotype of the cells. First a dual surface staining of the light chain and the Fc fragments was performed. While this staining did not all allow the detection of all produced species (no distinction between BEAT and Fab-DIM), it detected the monospecific contaminant scFv-Fc-DIM. This method is universally applicable for all kind of BEAT molecules. The second approach was more specific and required the soluble targets for the detection of the respective binding arms. In this case all three possible products were unequivocally detected by surface staining.

Recombinant pools and clones were generated by limiting dilution and flow cytometric cell sorting, respectively. Batch or fed batch cultivations were performed and the product accumulated in the supernatants was analysed by capillary electrophoresis. Eventually the secretion profile (BEAT, Fab-DIM or scFv-Fc-DIM fraction) measured in the supernatant was compared to the product profile displayed on the cell membrane of the recombinant cells.

### Material and Methods

### Establishment of Stable Pools

Stable transfected CHO cells were generated by co-transfection of the previously described vectors pGLEX41_BEAT-HC-I4-M1M2-M1M2-M1M2 (abbreviated as “pHC-M1M2”; SEQ ID NO: 50), pGLEX41_scFv-Fc-I4-M1M2-M1M2-M1M2 (abbreviated as “pscFv-Fc-M1M2”;SEQ ID NO: 52) and pGLEX41_BEAT-LC (abbreviated as “pLC”; SEQ ID NO:302) for the expression of the bispecific heterodimer BEAT together with two proprietary vectors for the expression of the protein providing resistance against the antibiotics puromycin and geneticin, respectively.

Suspension CHO-S cells were transfected with linearized vectors using polyethyleneimine (JetPEI®, Polyplus-transfection, Illkirch, France) in 50 ml bioreactor format. For this purpose, exponential growing cells were seeded at a density of 2 E6 cells/mL in 5 mL of OptiMEM medium (#31985-047, Invitrogen). A JetPEI®:DNA complex was added to the cells in a weight to weight ratio of 3 (μg/μg). Final DNA concentration of 2.5 μg/mL was added to the cells. After 5 hours incubation of the cells with the JetPEI®:DNA complex at 37° C. under shaking (200 rpm), 5 mL of culture medium PowerCHO2 (# BE12-771Q, Lonza) were added to the cell suspension. The cells were then incubated on a shaken platform at 37° C., 5% CO₂ and 80% humidity. One day after transfection the cells were seeded in 96 well plates at different concentration (at 0.7, 0.5 and 0.4 E5 cells/mL) in a selective medium containing puromycin (# P8833-25 mg, Sigma) and geneticin (#11811-098, Gibco) at a concentration allowing the growth of stable pools. After 14 days of selection under static conditions 15 producing stable pools were picked from the wells and expanded into TubeSpin bioreactors.

### Establishment of Recombinant Clones

Clones were generated by co-transfection of the previously described vectors pGLEX41_BEAT-HC-I4-B7-B7-B7 (abbreviated as “pHC-B7”; SEQ ID NO: 321), pGLEX41_scFv-Fc-I4-B7-B7-B7 (abbreviated as “pscFv-Fc-B7”; SEQ ID NO: 323) and pGLEX41_BEAT-LC (abbreviated as “pLC”; SEQ ID NO: 302) for the expression of the bispecific heterodimer BEAT together with two proprietary vectors for the expression of the protein providing resistance against the antibiotics puromycin and geneticin, respectively. Transfection and selection of stable transfectants were performed as previously described. After 14 days of selection under static conditions all stable transfectants were collected, pooled and passaged under dynamic conditions (orbital shaking) for a week. Single cell sorting was performed using a FACSAria II by gating on living cells excluding cell doublets by pulse processing.

### LC and Fc Staining of the Product Displayed on the Cell Surface

Dual surface staining of the light chain (LC) and the Fc fragment on the stable cells was performed on day 2 after batch seeding as described in detail in Example 2. In short, a total of 1 E5 cells were harvested and transferred to a round bottom well of a 96 well plate. The cells were washed twice with washing buffer (PBS containing 2% FBS) and then resuspended in 100 μL of detection antibody. The specific detection of the kappa light chain was performed using a mouse anti-human kappa LC APC labelled antibody (#561323, BD Pharmingen), Fc fragments were detected using PE conjugated goat anti-human Fc gamma specific antibody (#12-4998-82, eBioscience). After 20 min incubation in the dark at room temperature, the cells were washed once in washing buffer and resuspended in 200 μL for flow cytometric analysis. The cells were analysed with a Guava flow cytometer (Merck Millipore).

### Staining of the Specific Binders Displayed on the Cell Surface

For this staining the soluble targets were used for the detection of the binding arms of the molecule displayed on the cell surface. In short, a total of 1 E5 cells were harvested and transferred to a round bottom well of a 96 well plate. The cells were washed twice with washing buffer (PBS containing 2% FBS) and then resuspended in 100 μL of a mix of biotinylated Target1 and His-tagged Target2. After 20 min incubation in the dark at room temperature, the cells were washed twice with washing buffer and resuspended in 100 μL of a mix containing Streptavidin conjugated with APC (#554067, BD Pharmingen) and an anti His-Tag antibody labelled with PE (#IC050P, RD Systems). After 20 min incubation in the dark at room temperature, the cells were washed once in washing buffer and resuspended in 200 μL for flow cytometric analysis with a Guava flow cytometer (Merck Millipore).

### Analysis of the Cell Secretion Profile

Recombinant pools were seeded in TubeSpins at a cell density of 0.5 E6 cells/mL in supplemented growth medium (PowerCHO2, 2 mM L-Glutamine, 8 mM Glutamax (Life Technologies, Carlsbad, Calif.), 15% Efficient Feed A (Life Technologies), 15% Efficient Feed B (Life Technologies). Clones were seeded in TubeSpins at a cell density of 0.5 E6 cells/mL in growth medium (PowerCHO2, 2 mM L-Glutamine) and feeds of ActiCHO Feed A & B feed, (#U050-078, PAA Laboratories GmbH) were performed on a daily basis. On day 14 of the batch or fed-batches culture supernatants were harvested by centrifugation and filtered using a 0.2 μm syringe filter (#99722. TPP). The crude supernatants were either directly analysed or purified using Streamline Protein A beads (#17-1281-02, GE). The proteins were analysed using the Caliper LabChip GXII protein assay. The different species were identified according to their molecular weight and the fraction of each product was determined.

### Results

A total of 15 stable pools were obtained following transfection with pHC-M1M2, pScFv-Fc-M1M2 and pLC constructs. Supplemented batches were started in order to determine the secretion profile of these pools. Dual surface staining of the light chain and the Fc fragments (scFv-Fc and the heavy chain) on the pools was performed on day 2 after batch seeding. FIG. 19 gives an example of the surface profile of one of the pools measured by dual staining on day 2 of a supplemented batch. The dot plot presents the intensity for kappa LC staining (y-axis) versus the intensity of human Fc staining (x-axis). The quadrant fixes arbitrary limits between negatively and positively stained cells. Quadrant Q8 represents the fraction of cells expressing no protein on the cell membrane, e.g. a sub population of non-producers. Q7 is the fraction of positive cells for the Fc fragment but negative for kappa LC thus corresponding to cells exhibiting scFv-Fc-DIM on the cell membrane. Dual positive cells, in Q6, are the cells displaying both Lc and Fc fragments on the cell membrane, e.g. BEAT or Fab-DIM molecules.

The analysis indicated a heterogeneous cell population of 33.8% of non-producers (Q8), 50.3% of scFv-Fc-DIM secretors (Q7) and 15.8% of potential BEAT or Fab-DIM secretors (Q6). In order to be able to calculate a correlation between the surface staining and the secreted product, only the producing population was taken into account. Hence the fraction of BEAT and Fab-DIM producers as well as the fraction of scFv-Fc-DIM producers within the producing cell fraction (Q6+Q7) was re-calculated excluding the non-producer fraction Q8 (this fractions will be referred to as Q6* and Q7*). In the analysis shown in FIG. 17 for example the populations were calculated as follows:

Q6*=Q6/(Q6+Q7)=23.9% and Q7*=Q7/(Q6+Q7)=76.1%.

This analysis was performed for all 15 producing stable pools and used for the correlation shown in FIG. 20.

The fraction of BEAT, Fab-DIM and scFv-Fc-DIM producers could be identified by surface staining according to the species display on the cell membrane by alternate splicing. The results were then confronted with the actual secretion profile of the analysed pools. For this purpose, the supernatants of the 15 producing stable pools were purified on day 14 by Protein A and each secreted molecule was identified according to the molecular weight and quantified by Caliper LabChip GXII protein assay. It should be noted that under these purification conditions only the Protein A binding species, namely BEAT and scFv-Fc-DIM molecules, are purified from the supernatant as Fab-DIM molecules lack the Protein A binding site. FIG. 20 shows the relationship between the percentage of secreted molecules and the corresponding cell fraction identified by surface staining.

The data indicate that surface display and secretion profile are highly correlated (R²=0.9) for both BEAT and scFv-Fc-DIM secretion. The nature of the relationship is however not linear. The lack of linear regression may be explained by the fact that the surface staining performed in this experiment does not distinguish between BEAT and Fab-DIM, whereas the purified secreted products excludes the Fab-DIM fraction. The percentage of secreted BEAT and scFv-Fc-DIM in the supernatant may be slightly overestimated. Nevertheless, the experiment clearly demonstrated that the higher the fraction of positive cells for the surface display of a particular molecule, the higher is the fraction of this molecule in the supernatant. In this example, the fraction of secreted BEAT molecule increased linearly when the tested stable pools harboured a percentage of positive cells higher than 20%. The selection of a stable pool harbouring more than 75% of dual positive cells (Q6) would allow the selection of a secreting cell population yielding more than 90% of the desired BEAT molecule. For a clonal population harbouring a dual positive phenotype, e.g. 100% of the cell population positive for both LC and Fc surface staining, it is reasonable to expect an even higher fraction of BEAT secretion, approaching approximately 100%.

In order to increase the sensitivity of the method and demonstrate a linear relationship between surface display and the secretion pattern, a different detection method was applied involving the soluble target as detection reagents. This way all 3 possible products are unequivocally identified by their binding patterns. FIG. 21 gives an example of the surface profile of a pool measured prior cell sorting. The dot plot presents the cell surface density of the binder for Target1 (y-axis) versus the cell surface density of the binder for Target2 (x-axis). The quadrant fixes arbitrary limits between negatively and positively stained cells. Q1 is the fraction of positive cells for Target1 binding but negative for Target2 binding thus corresponding to cells exhibiting Fab-DIM on the cell membrane. Dual positive cells, in Q2, are the cells displaying both binders for Target1 and 2 on the cell membrane and thus correspond to cells displaying the BEAT on the membrane. Q3 are the cells displaying solely Target2 binders thus corresponding to scFv-Fc-DIM. Quadrant Q4 represents the fraction of cells expressing no protein on the cell membrane, e.g. a sub population of non-producers. As these cells did not contribute to the secretion of antibodies they were excluded from the analysis.

All producing clones (28) generated by flow cytometric cell sorting after transfection with pHC-B7, pScFv-Fc-B7 and pLC constructs were analysed using this approach. As previously described the fraction of BEAT, Fab-DIM and scFv-Fc-DIM producers could be identified by surface staining according to the species display on the cell membrane by alternate splicing. The actual secretion profiles of each clone were determined on day 14 of a fed batch directly in the supernatant by Caliper LabChip GXII protein assay.

FIG. 22 shows the correlation between the fraction of BEAT molecule detected in the supernatants and the fraction of cells displaying a BEAT phenotype on their cell surface (% of Q2). Using this specific staining a good linear correlation (R²=0.8) was obtained. The data clearly indicated that the aptitude of clone to secrete the BEAT molecule could be predicted by the product pattern detected on the cell membrane.

### CONCLUSION

In this example it was demonstrated that alternate splicing technology combined with cell membrane display is an effective reporter system for the qualitative prediction of the secretion profile of single cells. A clear correlation was found between the cell membrane display pattern and the actual secretion profile of transfected cells using either a general detection method based on LC and Fc surface detection or using a product specific staining method involving the soluble targets of the molecule. The approach does not require the time consuming screening of cell performances in batch or fed batch cultures, nor the harvest, purification and extensive characterization of secreted product.

In conclusion, the reporter system described here provides a reliable, high throughput screening tool for cell clones harbouring a particular secretion profile for heterotrimeric molecules.

In addition to the qualitative prediction of secretion profile, the quantitative prediction of the secretion level of particular cell clone is of high interest for cell line development purposes. In the following example it was investigated whether the level of membrane bound, surface displayed product using an alternate splicing approach correlated with the secretion level of a clone.

### Example 6: Quantitative Prediction of Secretion Level Using Alternate Splicing for Surface Display

### Introduction

A major challenge of the cell line development process is the selection in a time effective manner of high performing clones, for example, a high secretion rate of good quality recombinant protein. It was demonstrated previously that the display on the cell membrane of a fraction of an expressed protein via alternate splicing indicates the actual qualitative secretion profile of a clone. In this example it will be demonstrated that the level of cell membrane display correlates quantitatively with the secretion level of a clone and that high producing cells can be selected on the basis of the intensity of the surface display.

### Material and Methods

### Stable Cell Line Development

Stable transfected CHO cells were generated by co-transfection of the vectors pGLEX41_HC-I4-M1M2-M1M2-M1M2 (SEQ ID NO: 36) and pGLEX41_LC (SEQ ID NO: 294) for the expression of a humanized IgG1 antibody, together with two propriety vectors for the expression of the protein providing resistance against the antibiotics puromycin and geneticin, respectively.

Suspension CHO-S cells were transfected with linearized vectors using polyethyleneimine (JetPEI®, Polyplus-transfection, Illkirch, France) in 50 ml bioreactor format. For this purpose, exponential growing cells were seeded at a density of 2 E6 cells/mL in 5 mL of OptiMEM medium (#31985-047, Invitrogen). A JetPEI®:DNA complex was added to the cells in a weight to weight ratio of 3 (μg/μg). A final DNA concentration of 2.5 μg/mL was added to the cells. After 5 hours incubation of the cells with the JetPEI®:DNA complex at 37° C. under shaking (200 rpm), 5 mL of culture medium PowerCHO2 (# BE12-771Q, Lonza) was added to the cell suspension. The cells were then incubated on a shaken platform at 37° C., 5% CO₂ and 80% humidity. One day after transfection the cells were seeded in 96 well plates at different concentration (at 0.7, 0.5 and 0.4 E5 cells/mL) in a selective medium containing 4 jug/mL puromycin (# P8833-25 mg, Sigma) and 400 μg/mL geneticin (#11811-098, Gibco). After 14 days of selection under static conditions 15 producing stable pools were picked from the wells and expanded into TubeSpin bioreactors for the assessment of the expression level.

### Assessment of the Expression Level

Supplemented batches were seeded at a cell density of 0.5E6 cells/mL in PowerCHO2 (# BE12-771Q, Lonza) supplemented with 2 mM L-Glutamine, 8 mM Glutamax, 15% Efficient Feed A (#A1023401, Invitrogen) and 15% Efficient FeedB (#A1024001, Invitrogen). Viable cell count (VCC) and viability were monitored on day 1, 3 and 7 using the ViaCount assay of the Guava flow cytometer. IgG titers were measured on day 1, 3 and 7 using the octet QK instrument. The specific productivity qP of the stable pools was calculated between day 1 and day 3 according to the following formula

$$qP = \frac{Δ {IgG}_{d 3 - d 1}}{Δ {t}_{d 3 - d 1}} \times \frac{1}{{VCC}_{mean d 1 - d 3}}$$

### With:

qP=specific secretion rate or productivity [pg/cell/day]
IgG=IgG titers in [pg/mL] on day 3 and day 1
t=cultivation time [day]
VCC_{mean d1-d3}=mean viable cell count between day 1 and day 3 [cells/mL]

Quantification of the displayed IgGs on the cell membrane was performed on day 1 during the exponential growth phase as described in the following.

### Staining of the Cells

Staining of the Fc fragments on the stable pools was performed on day 1 after batch seeding. In short, a total of 1 E5 cells were harvested and transferred to a round bottom well of a 96 well plate. The cells were washed twice with the washing buffer (PBS containing 2% FBS) and resuspended in 100 μL of detection antibody. The specific detection of the Fc fragments was performed using a PE conjugated goat anti-human Fc gamma specific antibody (#12-4998-82, eBioscience). After 20 min incubation in the dark at room temperature, the cells were washed once in washing buffer and resuspended in 200 μL for flow cytometric analysis. The cells were analysed with a Guava flow cytometer.

### Flow Cytometric Cell Sorting

Clones were generated by flow cytometric cell sorting form a heterogeneous pool of stable transfectants established as previously described (see section “Stable cell line development”). For this purpose a surface staining of the Fc fragment was performed according to the protocol described earlier. The sort was performed using a FACSAria II by gating on living cells excluding cell doublets by pulse processing. Three gates were defined according to the surface fluorescence intensity (“low”, “medium” and “high”) and single cells were distributed into 96 well plates containing 200 μL of a cloning medium. After 2 weeks of growth under static condition the cells were expanded under dynamic conditions and the performances of the selected clones were evaluated as described previously.

### Results

The analysis of the surface staining profile of a stable pool is illustrated in FIG. 23. The surface fluorescence of living cells gated in the region g1 (FIG. 23, profile A) was displayed as a histogram (FIG. 23, plot B) and the mean fluorescence of the distribution (Mean [RFU]) was computed. The mean fluorescence is used as a measure of the IgG level expressed on the cell membrane. This analysis was performed for all generated stable pools and the level of surface IgG was compared to the actual level of secreted IgGs measured in the supernatant.

FIG. 24 shows the correlation between the surface fluorescence intensity and the secretion level of all stable pools measured on day 1 of a supplemented batch. A good correlation (R²>0.8) was observed between the surface IgG expression and the titers (FIG. 24, plot A) as well as between surface IgG expression and the specific productivity (FIG. 24, plot B). The data demonstrate that the surface expression of alternately spliced transmembrane IgG reported the actual secretion level of stable transfected cells. A similar good correlation (R²>0.8) was observed between the surface fluorescence of the pools on day 1 and the volumetric productivity in a 7 day batch process (see FIG. 25; in this batch day 7 corresponds to the end of the stationary phase). The data indicate that the performance of the pools at the end of a batch process could be predicted early on according to the detected surface IgG expression. In a batch process, the physiological state of the cells is continuously changing according to the changing medium environment. The correlation between surface display of reporter IgG via alternate splicing and the actual secretion level in a batch process is therefore independent of the physiological state of the cells (exponential growing or stationary) and the batch process phases (growth or production).

To validate the hypothesis clones were generated by flow cytometric cell sorting according to the density of antibody displayed on the cell surface. For this purpose a stable pool was stained for surface IgG using an anti-human Fc PE labelled antibody. Three regions were defined according to the surface fluorescence of the cells (“low”, “medium” and “high”) and the single cells were cloned accordingly in 96 well plates. The gating strategy can be seen in FIG. 26A. After approximately two weeks clones were expanded and the surface intensity of the cells was again determined to verify that indeed the sorted phenotype was obtained following cloning. The distribution of the surface intensity of all clones can be seen in FIG. 26B. Overall the sorting was successful as the distribution of the surface IgG of the three different sorted groups corresponded to the expected phenotype “low”, “medium” and “high”.

Some outliers were detected for instance in the “low” group showing a high surface fluorescence, probably due to an artefact of staining prior sorting. The performances of the generated clones were subsequently assessed in supplemented batches. FIG. 26C shows the distribution of the specific productivity qP for each group. The qP of the original pool was also assessed in quadruplicate and is given in the diagram as a control. The best performers were clearly identified within the “high” surface fluorescence group with a median qP of approx. 10 pg/cell/day. Compared to the original pool (approx. 5 pg/cell/day) the productivity was significantly improved by a 2-6-fold factor, the best clone reaching more than 30 pg/cell/day. In contrast the clones of the “low” and “medium” categories were overall low performers with a median qP close to 0 pg/cell/day. Some outliers were reaching >20 pg/cell/day but these phenotypes were also showing high surface density of IgG detected by flow cytometry.

### CONCLUSION

In this example it was demonstrated that the level of IgG expressed via alternate splicing on the cell membrane reports the actual secretion level of recombinant cells. The cell membrane fluorescence after the specific detection of the product correlates with the accumulated IgG concentration in the supernatant and the qP of transfected stable cells. Also it was demonstrated that the correlation was valid regardless of the phase of a batch production process. Taken together, these data indicated that the quantitative secretion level of recombinant cells can be predicted by the alternate splicing surface display reporter system described herein. This was verified by selecting clones according to the density of IgG displayed on the cell surface using flow cytometric cell sorting. Indeed, high producers could be selected using this approach and clones showing industrial relevant specific productivity could be generated in a time efficient manner.

1. An expression construct comprising in a 5′ to 3′ direction:
a promoter;
a first exon encoding a polypeptide of interest;
a splice donor site, an intron and a splice acceptor site, wherein a first stop codon is located between the splice donor site and the splice acceptor site within said intron;
a second exon encoding a transmembrane region which is selected from a modified immunoglobulin transmembrane region or a modified or unmodified non-immunoglobulin transmembrane region;
a second stop codon; and
a poly(A) site,
wherein upon entry into a host cell, transcription of said first and second exons results in the expression of the polypeptide of interest and the surface display of a proportion of the polypeptide of interest on the host cell membrane.

2. The expression construct according to claim 1, wherein said transmembrane region comprises between 17 and 29 residues, preferably 19 and 26 residues and most preferably between 21 and 24 residues.

3. An expression construct according to claim 1 or 2, wherein the non-immunoglobulin transmembrane region is selected from the group consisting of the transmembrane region of human platelet-derived growth factor receptor (PDGFR), human asialoglycoprotein receptor, human and murine B7-1, human ICAM-1, human erbb1, human erbb2, human erbb3, human erbb4, human fibroblast growth factor receptors such as FGFR 1,FGFR2, FGFR3, FGFR4, human VEGFR-1, human VEGFR-2, human erythropoietin receptor, human PRL-R, prolactin receptor, human EphA1, Ephrin type-A receptor 1, human insulin, IGF-1 receptors, human receptor-like protein tyrosine phosphatases, human neuropilin, human major histocompatibility complex class II (alpha and beta chains), human integrins (alpha and beta families), human Syndecans, human Myelin protein, human cadherins, human synaptobrevin-2, human glycophorin-A, human Bnip3, human APP, amyloid precursor protein, human T-cell receptor alpha and beta, CD3 gamma, CD3 delta, CD3 zeta, and CD3 epsilon.

4. An expression construct according to claim 1, wherein the non-immunoglobulin transmembrane region is the murine B7-1 transmembrane region (SEQ ID NO: 173), ACLV1 (SEQ ID NO: 174), ANTR2 (SEQ ID NO: 175), CD4 (SEQ ID NO: 176), PTPRM (SEQ ID NO: 177), TNR5 (SEQ ID NO: 178), ITB1 (SEQ ID NO: 179), IGF1R (SEQ ID NO: 181), 1B07 (SEQ ID NO: 180), TRMB (SEQ ID NO: 182), IL4RA (SEQ ID NO: 183), LRP6 (SEQ ID NO: 184), GpA (SEQ ID NO: 185), PTCRA (SEQ ID NO: 186).

5. An expression construct according to any one of the preceding claims, wherein said intron comprises a poly(A) site.

6. An expression construct according to any one of the preceding claims, wherein the splice acceptor site of said intron comprises a poly(Y) tract and wherein the Y content of the poly(Y) tract is modified by altering the number of pyrimidine bases therein.

7. An expression construct according to any one of the preceding claims comprising at least one branch point region, wherein the sequence of said at least one branch point region is modified with respect to the branch point region consensus sequence CTRAYY (SEQ ID NO: 347).

8. An expression construct according to any one of the preceding claims, wherein the consensus sequence of the splice donor site of the intron is modified.

9. An expression construct according to any one of the preceding claims, wherein the polypeptide of interest comprises an antibody heavy chain or fragment thereof.

10. A polynucleotide encoding an expression construct according to any one of the preceding claims.

11. A cloning or expression vector comprising one or more polynucleotides according to claim 10.

12. A host cell comprising one or more cloning or expression vectors according to claim 11.

13. A host cell according to claim 12 comprising an expression vector comprising a polynucleotide encoding the expression construct of claim 9 for the expression of an antibody heavy chain and an expression vector comprising a polynucleotide encoding an expression construct for the expression of an antibody light chain.

14. A host cell according to claim 12 comprising an expression vector comprising a polynucleotide encoding the expression construct of claim 9 for the expression of an antibody heavy chain, an expression vector comprising a polynucleotide encoding the expression construct of claim 8 for the expression of a scFv-Fc and an expression vector comprising a polynucleotide encoding an expression construct for the expression of an antibody light chain.

15. A method of producing a polypeptide comprising culturing the host cell(s) of any one of claims 12 to 14 in a culture and isolating the polypeptide expressed from the culture.

16. A method of selecting a host cell(s) expressing a polypeptide of interest comprising:
(i) transfecting a host cell(s) with an expression construct of claim 1;
(ii) culturing the host cell(s) under conditions suitable to express the polypeptide of interest;
(iii) detecting cell membrane expression of the polypeptide of interest; and
(iv) selecting the host cell(s) displaying the polypeptide of interest on the surface of the cell membrane at the desired expression level.

17. A method of selecting a host cell(s) expressing a heteromultimeric polypeptide of interest comprising:
(i) co-transfecting a host cell(s) with at least an expression construct of claim 1 encoding an a first polypeptide of interest and an expression construct of claim 1 encoding a second polypeptide of interest;
(ii) culturing the host cell(s) under conditions suitable to express the heteromultimeric polypeptide of interest;
(iii) detecting cell membrane expression of the heteromultimeric polypeptide of interest; and
(iv) selecting the host cell(s) displaying the desired heteromultimeric polypeptide of interest on the surface of the cell membrane at the desired expression level.

18. A method of selecting a host cell(s) expressing a bispecific antibody comprising:
(i) co-transfecting a host cell(s) with an expression construct of claim 1 encoding an antibody heavy chain, an expression construct of claim 1 encoding a scFv-Fc and an expression construct encoding an antibody light chain;
(ii) culturing the host cell(s) under conditions suitable to express the bispecific antibody;
(iii) detecting cell membrane expression of the bispecific antibody; and
(iv) selecting the host cell(s) displaying the desired bispecific antibody on the surface of the cell membrane at the desired expression level.
